(19) United States
(12) Patent Application Publication
(10) Pub. No.: US 2020/0316100 A1
(43) Pub. Date: Oct. 8, 2020

(54) HALOALKYL FUCOSE-CONTAINING SELECTIN ANTAGONISTS

(71) Applicant: GlycoMimetics, Inc., Rockville, MD (US)

(72) Inventors: Beat ERNST, Magden (CH); Norbert VARGA, Allschwil (CH); Martin SMIESKO, Allschwl (CH); Beatrice WAGNER, Aesch BL (CH); John M. PETERSON, Slate Hill, NY (US); John L. MAGNANI, Gaithersburg, MD (US)

(73) Assignee: GycoMimetics, Inc., Rockville, MD (US)

(21) Appl. No.: 16/303,852

(22) PCT Filed: May 22, 2017
(86) PCT No.: PCT/US2017/033793

§ 371 Date: Nov. 21, 2018

Related U.S. Application Data
(60) Provisional application No. 62/340,770, filed on May 24, 2016.

(51) Int. Cl. A61K 31/7052; C07H 15/26; A61N 5/10

(52) U.S. Cl. CPC A61K 31/7052; A61N 2005/1098; A61N 5/10; C07H 15/26

(57) ABSTRACT

Compounds, compositions, and methods for treatment and/or prevention of at least one disease, disorder, and/or condition by inhibiting binding of an E-selectin to an E-selectin ligand are disclosed. For example, haloalkyl fucose-containing E-selectin antagonists and compositions comprising at least one such agent are described.

This application claims the benefit under 35 U.S.C. § 119(e) to U.S. Provisional Application No. 62/340,770 filed May 24, 2016, which application is incorporated by reference herein in its entirety.

Compounds, compositions, and methods for treating and/or preventing at least one disease, disorder, and/or condition associated with E-selectin activity including, for example, inflammatory diseases and cancers, are disclosed herein.

When a tissue is infected or damaged, the inflammatory process directs leukocytes and other immune system components to the site of infection or injury. Within this process, leukocytes play an important role in the engulfment and digestion of microorganisms. The recruitment of leukocytes to infected or damaged tissue is critical for mounting an effective immune defense.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic membrane spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-thediated intercellular adhesion is an example of a selectin-mediated function.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium. In such circumstances, modulation of selectin-mediated intercellular adhesion may be desirable

Modulators of selectin-mediated function include the PSGL-1 protein (and smaller peptide fragments), fucoidan, glycyrrhizin (and derivatives), sulfated lactose derivatives, heparin and heparin fragments, sulfated hyaluronic acid, condroitin sulfate, sulfated dextran, sulfatides, and particular glycomimetic compounds (see, e.g., US RE44,778). To date, all but the glycomimetics have shown to be unsuitable for drug development as selectin antagonists due to insufficient activity, toxicity, lack of specificity, poor ADME characteristics, and/or availability of material.

Accordingly, there is a need in the art for identifying inhibitors of selectin-mediated function, e.g., of selectin-dependent cell adhesion, and for the development of methods employing such compounds. The present disclosure may fulfill one or more of these needs and/or may provide other advantages.

Compounds, compositions, and methods for treating and/or preventing (i.e., reducing the likelihood of occurrence or reoccurance) at least one disease, disorder, and/or condition in which inhibiting binding of E-selectin to one or more E-selectin ligands may play a role are disclosed.

Disclosed are glycomimetic E-selectin antagonists of Formula (I):

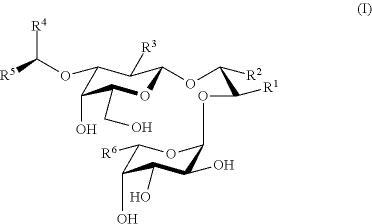

prodrugs of Formula (I), and pharmaceutically acceptable salts of any of the foregoing, wherein R¹, R², R³, R⁴, R⁵, and R⁶ are defined herein.

As used herein, ‘compound of Formula (I)’ includes E-selectin antagonists of Formula (I), pharmaceutically acceptable salts of E-selectin antagonists of Formula (I), prodrugs of E-selectin antagonists of Formula (I), and pharmaceutically acceptable salts of prodrugs of E-selectin antagonists of Formula (I).

In some embodiments, pharmaceutical compositions comprising at least one compound of Formula (I) and optionally at least one additional pharmaceutically acceptable ingredient are presented.

In some embodiments, a method for treatment and/or prevention of at least one disease, disorder, and/or condition where inhibition of E-selectin mediated functions is useful is disclosed, the method comprising administering to a subject in need thereof an effective amount of at least one compound of Formula (I) or a pharmaceutical composition comprising at least one compound of Formula (I).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the general reaction scheme for synthesizing exemplary embodiments of glycomimetic E-selectin antagonists of Formula (I).

FIG. 2 is a diagram illustrating the synthesis of building block 11.

FIG. 3 is a diagram illustrating the synthesis of intermediates 13, 17, and 20.

FIG. 4 is a diagram illustrating the synthesis of compounds 27 and 28.

FIG. 5 is a diagram illustrating the synthesis of compound 33.

FIG. 6 is a diagram illustrating the synthesis of compounds 37 and 38.

FIG. 7 is a diagram illustrating the synthesis of compounds 41 and 42.

FIG. 8 is a diagram illustrating the synthesis of building blocks 44, 45, and 47.

FIG. 9 is a diagram illustrating the synthesis of compounds 51 and 53.

FIG. 10 is a diagram illustrating the synthesis of compound 59.

FIG. 11 is a diagram illustrating the synthesis of compound 69.

FIG. 12 is a diagram illustrating an alternative synthesis of building block 10.

FIG. 13 is a diagram illustrating the synthesis of compound 75

Disclosed herein are glycomimetic E-selectin antagonists, pharmaceutical compositions comprising the same, and methods for inhibiting E-selectin-mediated functions using the same. The compounds and compositions of the present disclosure may be useful for treating and/or preventing at least one disease, disorder, and/or condition that is treatable by inhibiting binding of E-selectin to one or more E-selectin ligands.

The compounds of the present disclosure may have at least one improved physicochemical, pharmacological, and/or pharmacokinetic property.

In some embodiments, presented are glycotnitnetic E-selectin antagonists of Formula (I):

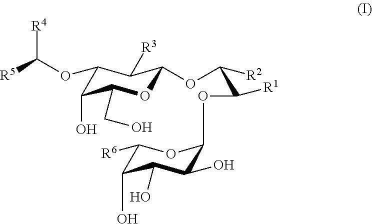

prodrugs of Formula (I), and pharmaceutically acceptable salts of any of the foregoing, wherein

R¹ and R², which may be identical or different, are independently chosen from H, C₁₋₁₂ alkyl, C₂₋₁₂ alkenyl, C₂₋₁₂ alkynyl, and C₇₋₁₄ arylalkyl groups, wherein R¹ and R² may join together along with the carbon atoms to which they are attached to form a ring having the formula:

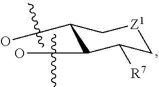

wherein Z¹ is chosen from —CH₂— and and wherein R⁷ is chosen from H, C₁₋₈ alkyl, and C₇₋₁₂ arylalkyl groups;

R³ is chosen from H, —OH, F, Cl, C₁₋₁₂ alkyl, —OY¹, and —OC(═O)Y¹ groups, wherein Y¹ is chosen from C₁₋₁₂ alkyl, C₂₋₁₂ alkenyl, C₂₋₁₂ alkynyl, and C₆₋₁₈ aryl groups;

R⁴ is chosen from —CN, —CH₂CN, —S(═O)₂Y², —C(S)Y², and —C(═O)Y² groups, wherein Y² is chosen from C₁₋₈ alkyl, C₂₋₈ alkenyl, C₂₋₈ alkynyl, —OZ², —NHOH, —NHOCH₃, —NHCN, —NHNH₂, and —NZ²Z³ groups, wherein Z² and Z³, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z² and Z³ may join together along with the nitrogen atom to which they are attached to form a ring;

R⁵ is chosen from C₁₋₁₈ alkyl, C₇₋₁₈ arylalkyl, and C₂₋₁₈ alkoxyalkyl groups; and

R⁶ is chosen from C₁₋₄ haloalkyl groups.

In some embodiments, R¹ is chosen from H and C₁₋₁₂ alkyl groups. In some embodiments, R¹ is chosen from H and C₁₋₆ alkyl groups. In some embodiments, R¹ is H. In some embodiments, R¹ is chosen from C₁₋₆ alkyl groups. In some embodiments, R¹ is chosen from C₁₋₄ alkyl groups. In some embodiments R¹ is chosen from methyl, ethyl, propyl, and butyl groups. In some embodiments, R¹ is chosen from C₇₋₁₄ arylalkyl groups. In some embodiments, R¹ is chosen from C₇₋₁₀ arylalkyl groups.

In some embodiments, R² is chosen from H and C₁₋₁₂ alkyl groups. In some embodiments, R² is chosen from H and C₁₋₆ alkyl groups. In some embodiments, R² is H. In some embodiments, R² is chosen from C₁₋₆ alkyl groups. In some embodiments, R² is chosen from C₁₋₄ alkyl groups. In some embodiments R² is chosen from methyl, ethyl, propyl, and butyl groups. In some embodiments, R² is chosen from C₇₋₁₄ arylalkyl groups. In some embodiments, R² is chosen from C₇₋₁₀ arylalkyl groups.

In some embodiments, R¹ is H and R² is chosen from C₁₋₆ alkyl groups. In some embodiments, R¹ is H and R² is methyl. In some embodiments, R¹ is chosen from C₁₋₆ alkyl groups and R² is H. In some embodiments, R¹ is methyl and R² is H. In some embodiments, each of R¹ and R² is H. In some embodiments, each of R¹ and R² is methyl.

In some embodiments, R¹ and R² join together along with the carbon atoms to which they are attached to form

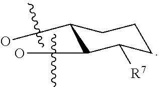

In some embodiments, R¹ and R² join together along with the carbon atoms to which they are attached to form

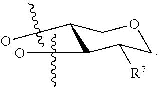

In some embodiments, R⁷ is chosen from H, C₁₋₈ alkyl, and C₇₋₁₂ arylalkyl groups. In some embodiments, R⁷ is H. In some embodiments, R⁷ is chosen from C₁₋₈ alkyl groups. In some embodiments, R⁷ is chosen from methyl and ethyl. In some embodiments, R⁷ is methyl. In some embodiments, R⁷ is ethyl. In some embodiments, R⁷ is chosen from C₇₋₁₂ arylalkyl groups. In some embodiments, R⁷ is chosen from C₇₋₁₀ arylalkyl groups. In some embodiments, R⁷ is 3-phenylpropyl. In some embodiments, R⁷ is phenethyl. In some embodiments, R⁷ is benzyl.

In some embodiments, R¹ and R² join together along with the carbon atoms to which they are attached to form a ring chosen from

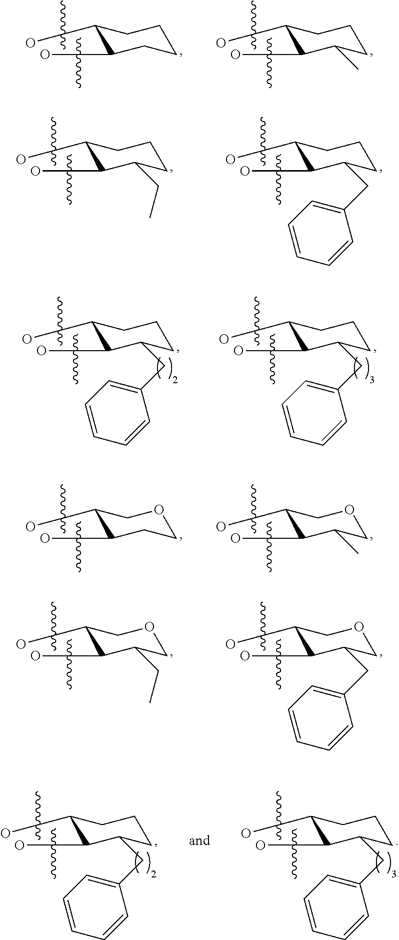

In some embodiments, R¹ and R² join together along with the carbon atoms to which they are attached to form

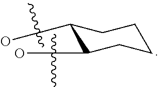

In some embodiments, R¹ and R² join together along with the carbon atoms to which they are attached to form

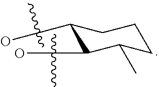

In some embodiments, R¹ and R² join together along with the carbon atoms to which they are attached to form

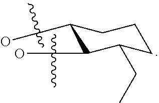

In some embodiments, R¹ and R² join together along with the carbon atoms to which they are attached to form

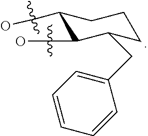

In some embodiments, R¹ and R² join together along with the carbon atoms to which they are attached to form

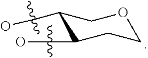

In some embodiments, R¹ and R² join together along with the carbon atoms to which they are attached to form

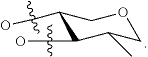

In some embodiments, R¹ and R² join together along with the carbon atoms to which they are attached to than

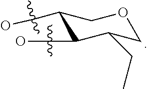

In some embodiments, R¹ and R² join together along with the carbon atoms to which they are attached to form

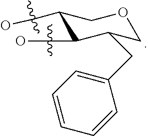

In some embodiments, R³ is chosen from H, —OH, F, and Cl. In some embodiments, R³ is H. In some embodiments, R³ is —OH. In some embodiments, R³ is F. In some embodiments, R³ is Cl. In some embodiments, R³ is chosen from —OY¹ and —OC(═O)Y¹ groups. In some embodiments, R³ is chosen from —OY¹ groups. In some embodiments, R³ is chosen from —O(═O)Y¹ groups. In some embodiments, Y¹ is chosen from C₁₋₁₂ alkyl groups. In some embodiments, Y¹ is chosen from C₆₋₁₈ aryl groups. In some embodiments, Y¹ is chosen from methyl, ethyl, propyl, isopropyl, and phenyl. In some embodiments, Y¹ is methyl. In some embodiments, Y¹ is ethyl, In some embodiments, Y¹ is propyl. In some embodiments, Y¹ is isopropyl. In some embodiments, Y¹ is phenyl.

In some embodiments, R⁴ is chosen from —CN and —CH₂CN. In some embodiments, R⁴ is chosen from —C(═)Y² groups. In some embodiments, Y² is chosen from C₁₋₈ alkyl, C₂₋₈ alkenyl, and C₂₋₈ alkynyl groups. In some embodiments, Y² is chosen from C₁₋₈ alkyl groups. In some embodiments, R⁴ is chosen from —S(═O)₂Y² and —C(═S)Y² groups. In some embodiments, R⁴ is chosen from S(═O)₂Y² groups. In some embodiments, R⁴ is chosen from —C(═S)Y² groups.

In some embodiments, R⁴ is chosen from —C(═O)OZ². In some embodiments, R⁴ is chosen from —C(═O)NHOH, —C(═O)NHOCH₃, —C(═O)NHNH₂, and —C(═O)NHCN. In some embodiments; R⁴ is chosen from —C(═O)NZ²Z³ groups. In some embodiments, Z² and Z³, which may be identical or different, are independently chosen from H, C₁₋₈ alkyl, C₁₋₈ haloalkyl, and C₇₋₁₂ arylalkyl groups. In some embodiments, at least one of Z² and Z³ is H. In some embodiments, each of Z² and Z³ is H. In some embodiments, at least one of Z² and Z³ is methyl. In some embodiments, each of Z² and Z³ is methyl. In some embodiments, at least one of Z² and Z³ is ethyl. In some embodiments, each of Z² and Z³ is ethyl. In some embodiments, Z² is and Z³ is methyl. In some embodiments, Z² and Z³ join together along with the nitrogen atom to which they are attached to form a ring.

In some embodiments, R⁴ is chosen from

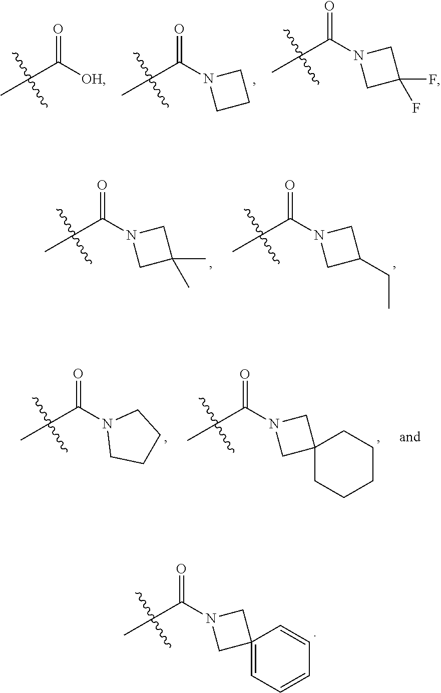

In some embodiments, R⁴ is chosen from

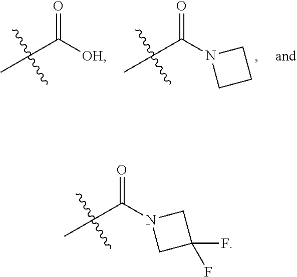

In some embodiments, R⁴ is

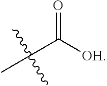 [SMILES: *C(=O)O]

In some embodiments, R⁴ is

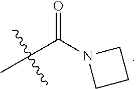 [SMILES: *C(=O)N1CCC1]

In some embodiments, R⁴ is

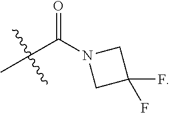 [SMILES: *C(=O)N1CC(F)(F)C1]

In some embodiments, R⁵ is chosen from C₁₋₁₈ alkyl groups. In some embodiments, R⁵ is chosen from C₁₋₁₂ alkyl groups. In some embodiments, R⁵ is chosen from C₁₋₈ alkyl groups. In some embodiments, R⁵ is chosen from C₇₋₁₈ arylalkyl groups. In some embodiments, R⁵ is chosen from C₇₋₁₄ arylalkyl groups. In some embodiments, R⁵ is chosen from C₇₋₁₀ arylalkyl groups. In some embodiments, R⁵ is chosen from C₂₋₁₈ is alkoxyalkyl groups. In some embodiments, R⁵ is chosen from C₂₋₁₂ alkoxyalkyl groups. In some embodiments, R⁵ is chosen from C₂₋₈ alkoxyalkyl groups. In some embodiments, R⁵ is chosen from C₂₋₄ alkoxyalkyl groups.

In some embodiments, R⁵ is chosen from:

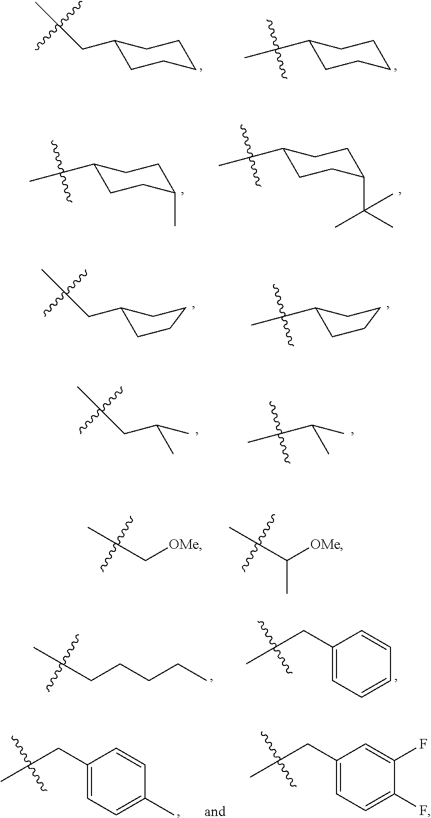

In some embodiments, R⁵ is chosen from:

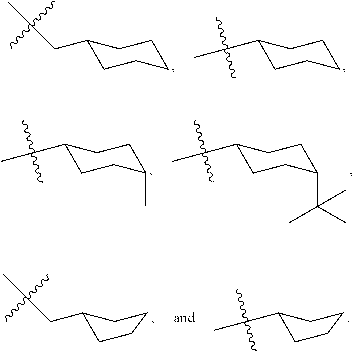

In some embodiments, R⁵ is chosen from:

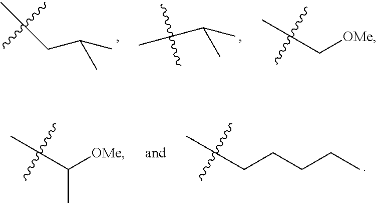

In some embodiments, R⁵ is chosen from:

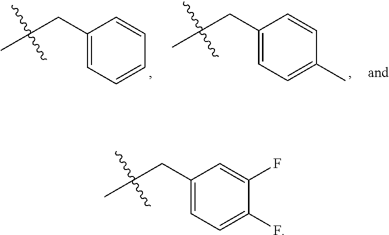

In some embodiments, R⁵ is

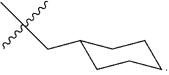 [SMILES: *CC1CCCCC1]

In some embodiments, R⁶ is chosen from halomethyl groups. In some embodiments, R⁶ is chosen from CH₂F, CHF₂, and CF₃. In some embodiments, R⁶ is chosen from CH₂Cl, CHCl₂, and CCl₃. In some embodiments, R⁶ is CF₃.

In some embodiments, at least one compound is chosen from compounds having the following Formula:

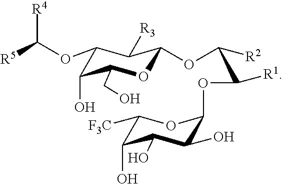

In some embodiments, at least one compound is chosen from compounds of the following Formulae:

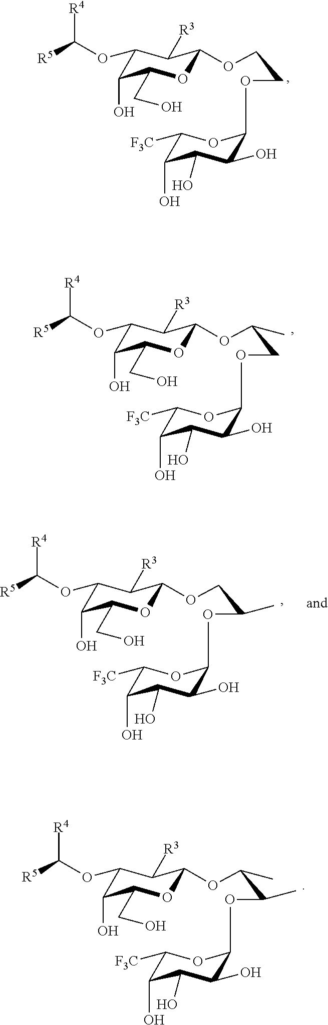

In some embodiments, at least one compound is chosen from compounds of the following Formulae:

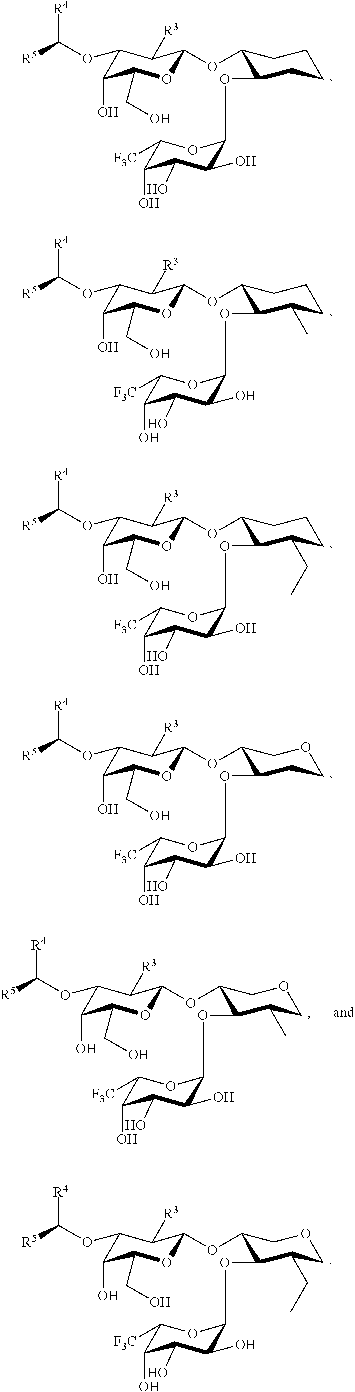

In some embodiments, at one compound is chosen from compounds having the following Formula:

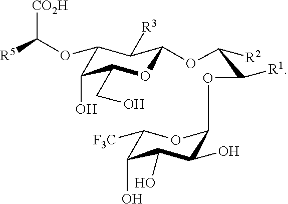

In some embodiments, at least one compound is chosen from compounds of the forming Formulae:

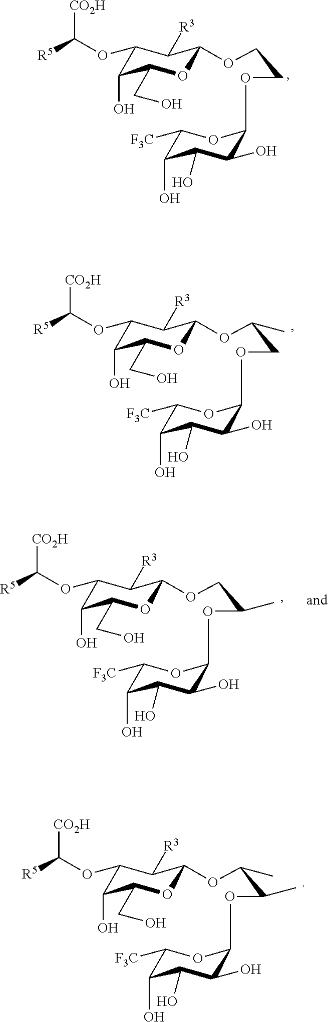

In some embodiments, at least one compound is chosen from compounds of the following Formulae:

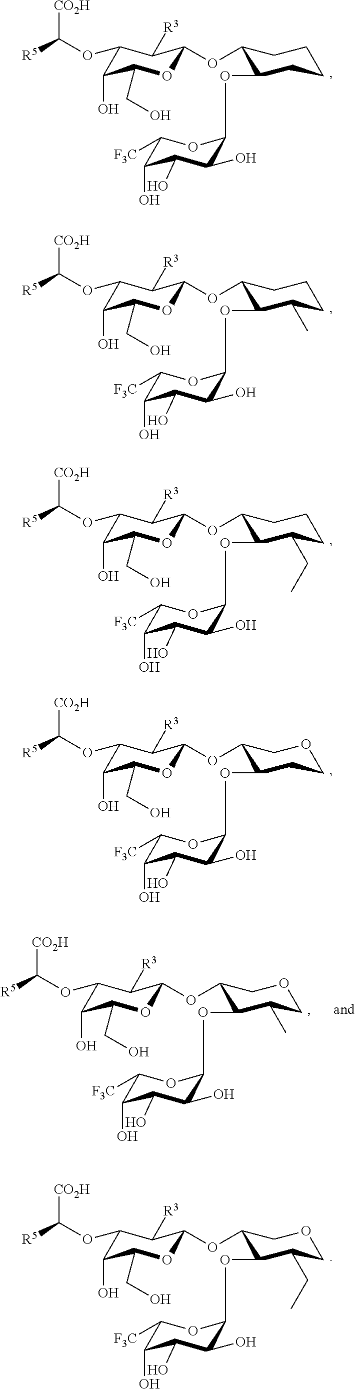

In some embodiments, at least one compound is chosen from compounds having the following Formula:

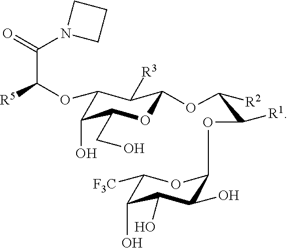

In some embodiments, at least one compound is chosen from compounds of the following Formulae:

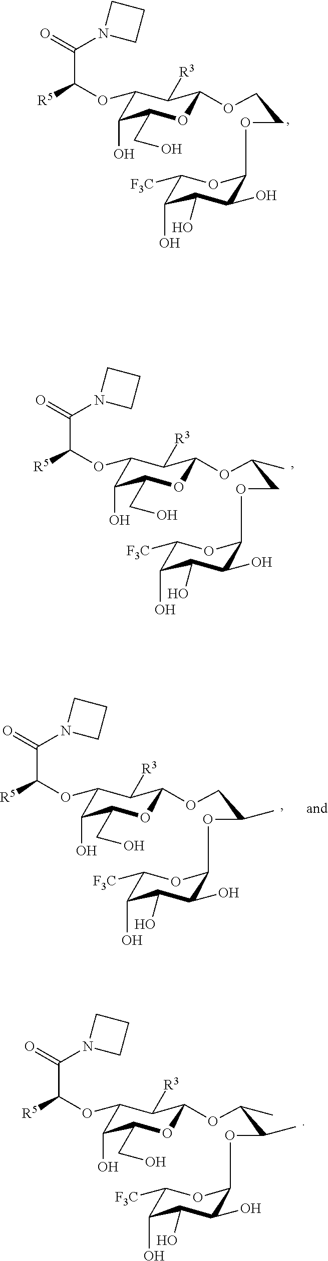

In some embodiments, at least one compound is chosen from compounds of the following Formulae:

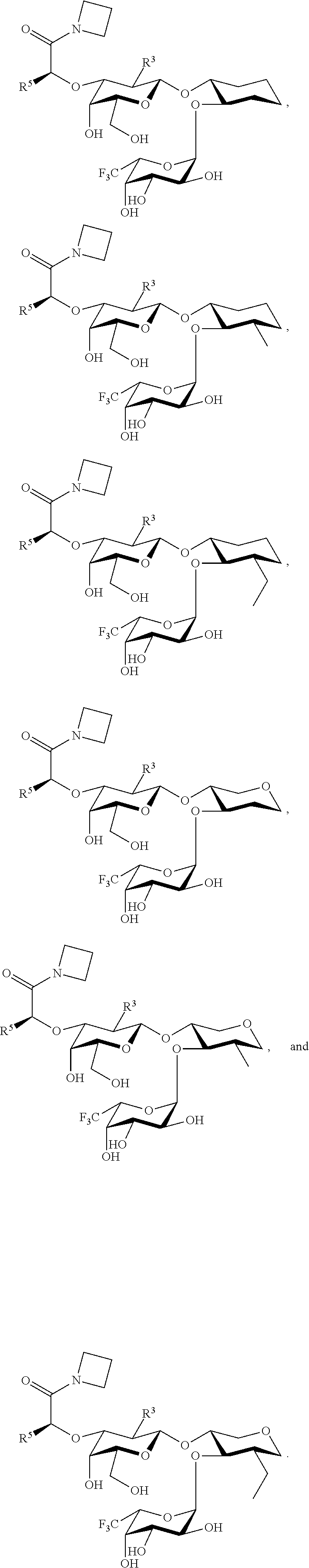

In some embodiments, at least one compound is chosen from compounds having the following Formula:

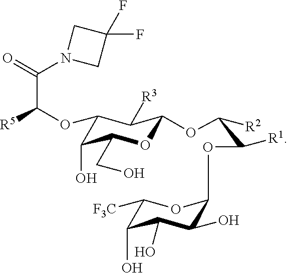

In some embodiments, at least one compound is chosen from compounds of the following Formulae:

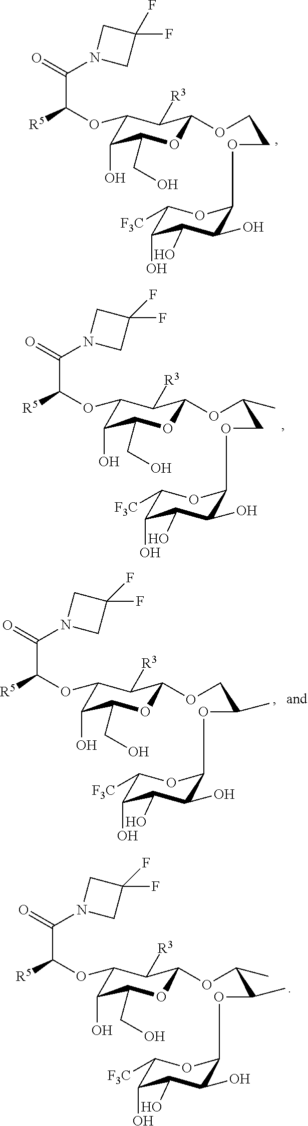

In some embodiments, at least one compound is chosen from compounds of the following Formulae:

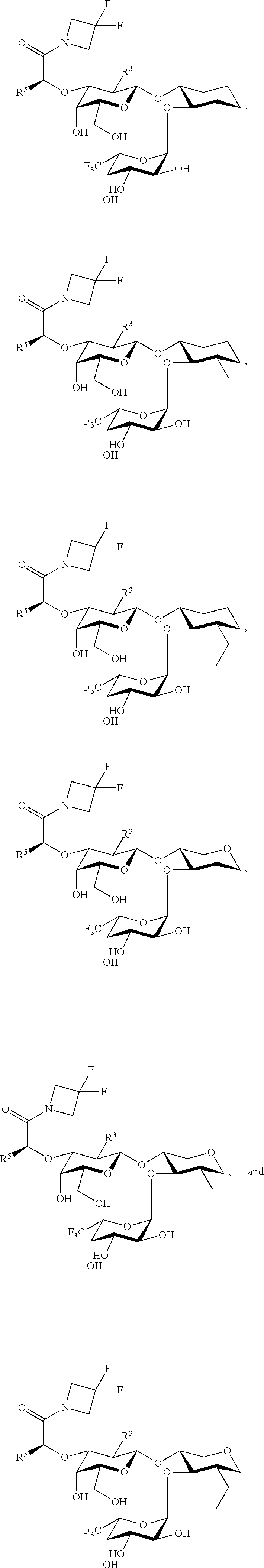

In some embodiments, at least one compound is chosen from compounds having the following Formula:

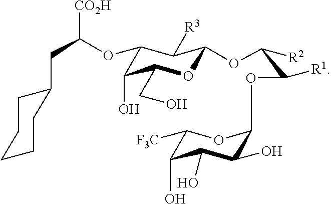

In some embodiments, at least one compound is chosen from compounds of the following Formulae:

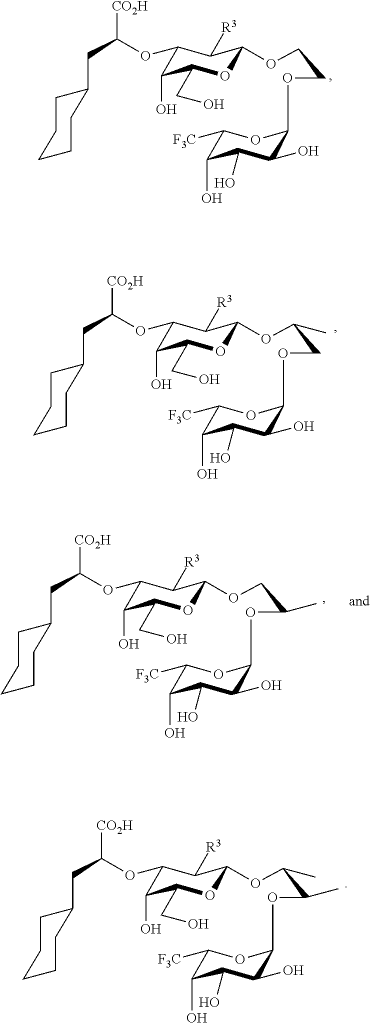

In some embodiments, at least one compound is chosen from compounds of the following Formulae:

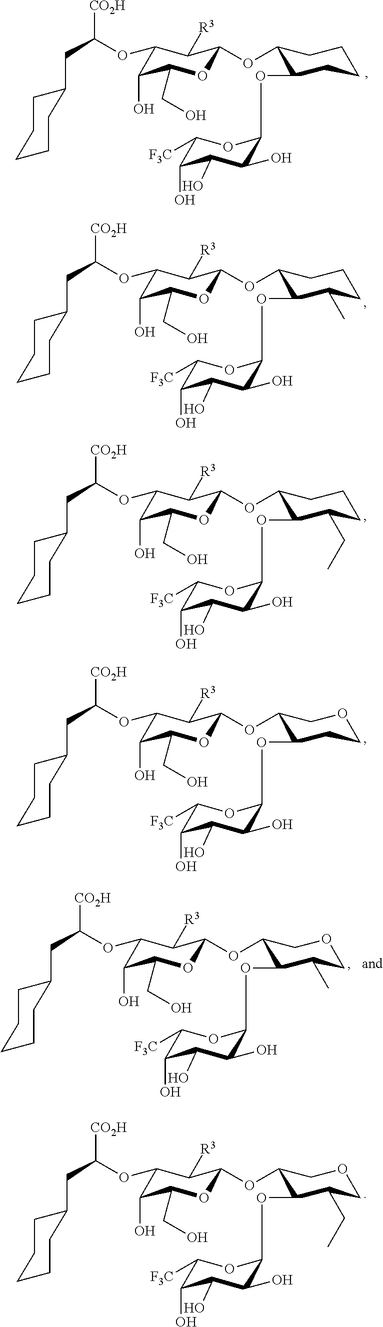

In some embodiments, at least one compound is chosen from compounds having the following Formula:

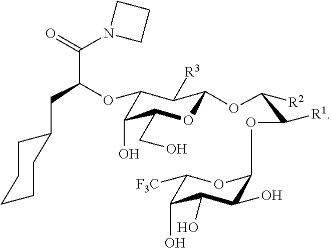

In some embodiments, at least one compound is chosen from compounds of the following Formulae:

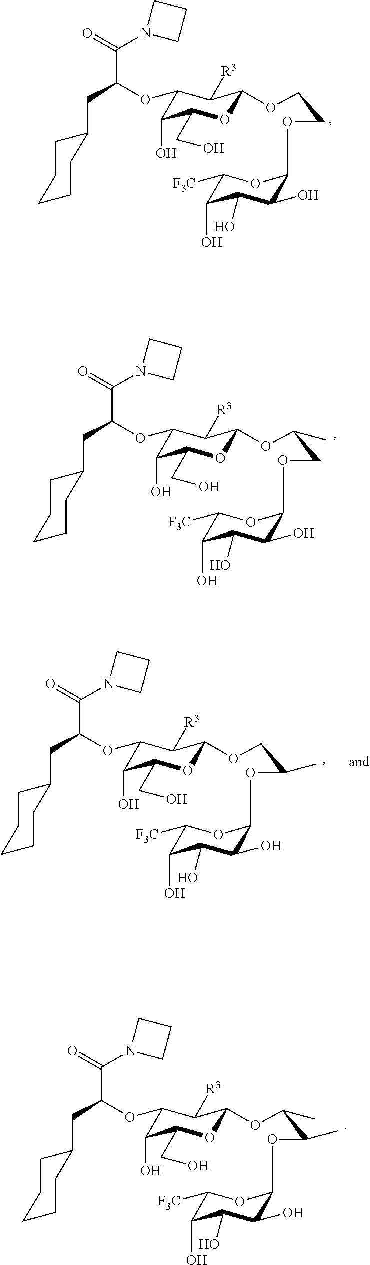

In some embodiments, at least one compound is chosen from compounds of the following Formulae:

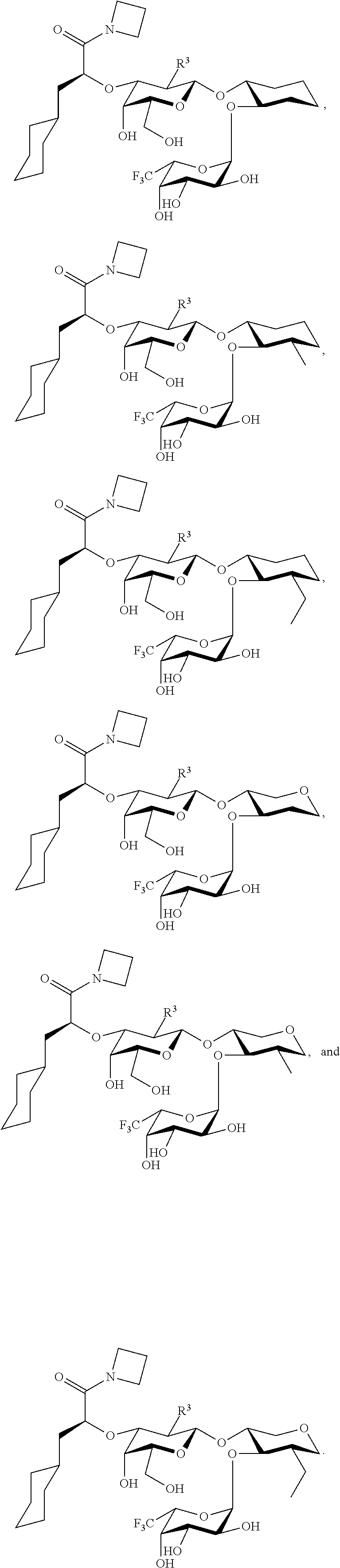

In some embodiments, at least one compound is chosen from compounds having the following Formula:

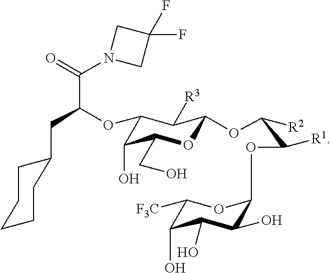

In some embodiments, at least one compound is chosen from compounds of the following Formulae:

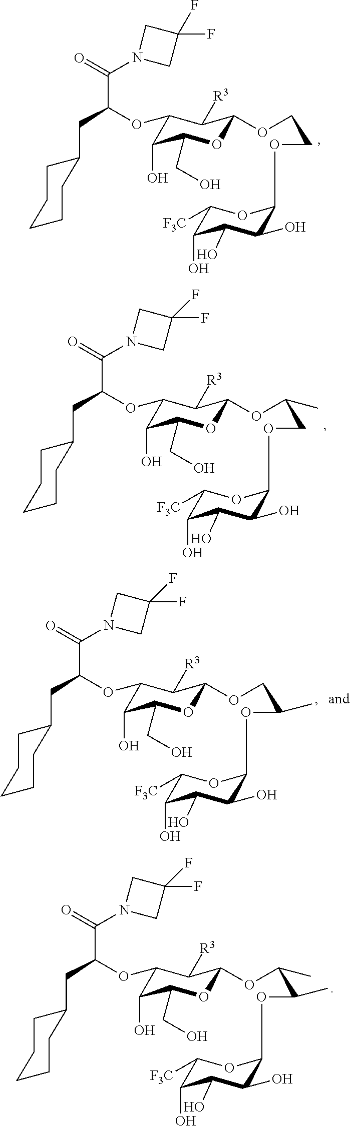

In some embodiments, at least one compound is chosen from compounds of the following Formulae:

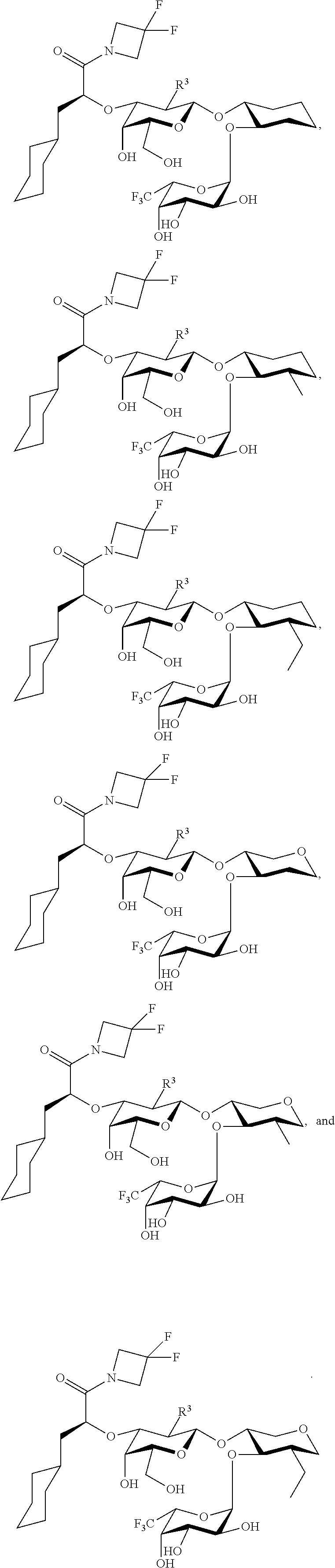

In some embodiments, at least one compound is chosen from compounds having the following Formulae:

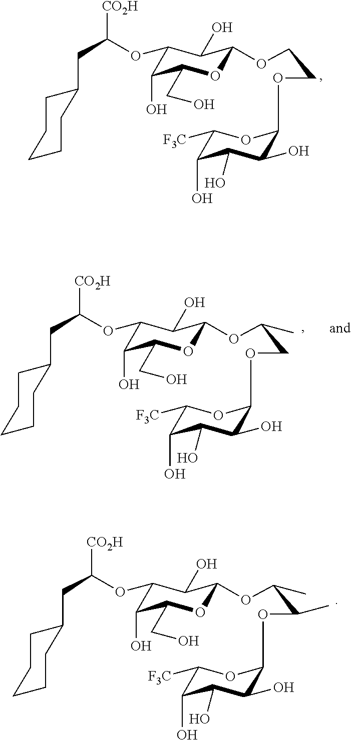

In some embodiments, at least one compound is chosen from compounds having the following Formulae:

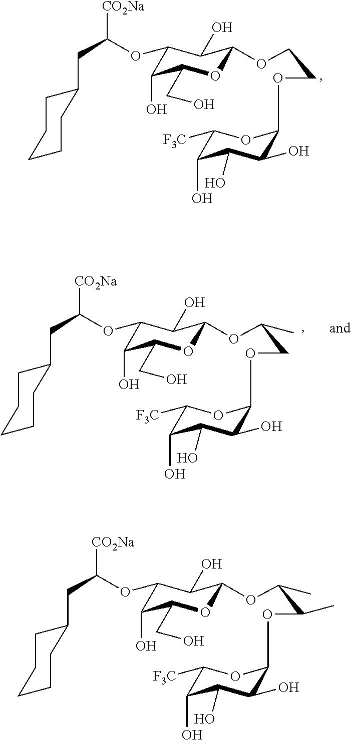

In some embodiments, at least one compound is chosen from compounds having the following Formulae:

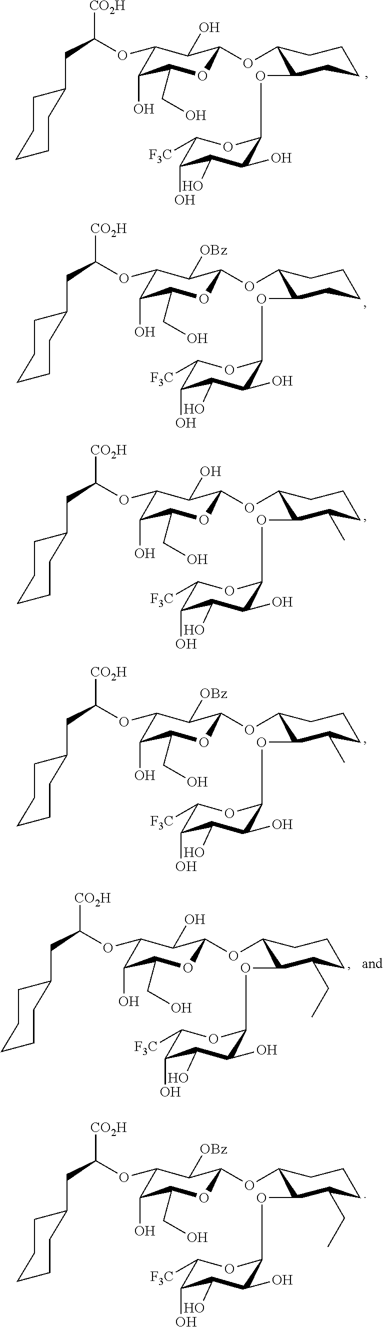

In some embodiments, at least one compound is chosen from compounds having the following Formulae:

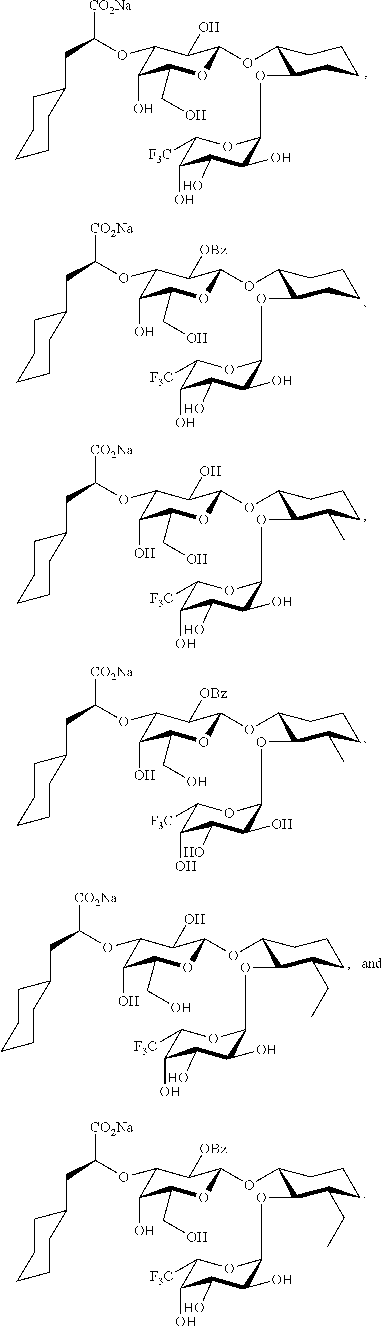

In some embodiments, at least one compound is chosen from compounds having the following Formulae:

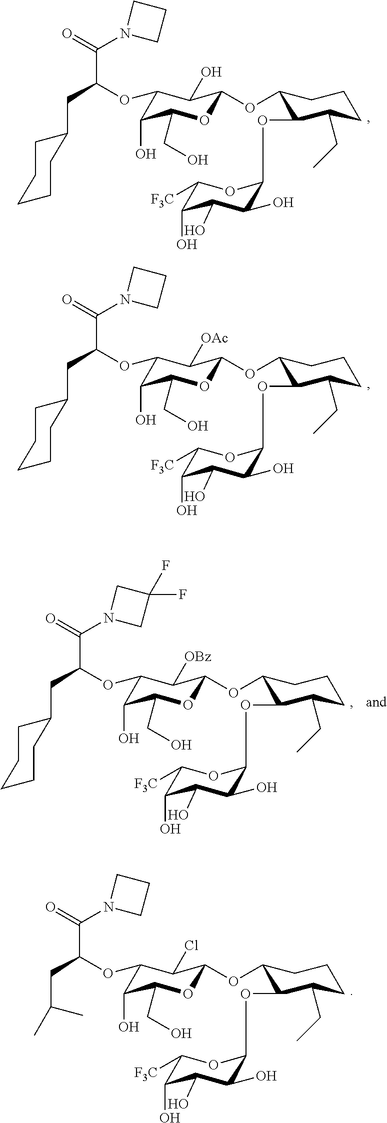

Also provided are pharmaceutical compositions comprising at least one compound of Formula (I). Such pharmaceutical compositions are described in greater detail herein. These compounds and compositions may be used in the methods described herein.

In some embodiments, a method for a treating and/or preventing at least one disease, disorder, and/or condition where inhibition of E-selectin mediated functions may be useful is disclosed, the method comprising administering at least one compound of Formula and/or a pharmaceutical composition comprising at least one compound of Formula (I).

In some embodiments, a method for treating and/or preventing at least one inflammatory disease, disorder, and/or condition in which the adhesion and/or migration of cells occurs in the disease, disorder, and/or condition is disclosed, the method comprising administering at least one compound of Formula (I) and/or a pharmaceutical composition comprising at least one compound of Formula (I).

In some embodiments, a method for inhibiting adhesion of a cancer cell that expresses a ligand of E-selectin to an endothelial cell expressing E-selectin on the cell surface of the endothelial cell is disclosed, the method comprising contacting the endothelial cell and at least one compound of Formula (I) and/or a pharmaceutical composition comprising at least one compound of Formula (I) such that the at least one compound of Formula (I) interacts with E-selectin on the endothelial cell, thereby inhibiting binding of the cancer cell to the endothelial cell. in some embodiments, the endothelial cell is present in the bone marrow.

In some embodiment, a method for treating and/or preventing a cancer is disclosed, the method comprising administering to a subject in need thereof an effective amount of at least one compound of Formula (I) and/or a pharmaceutical composition comprising at least one compound of Formula (I). In some embodiments, at least one compound of Formula (I) and/or pharmaceutical composition comprising at least one compound of Formula (I) may be administered in conjunction with (i.e., as an adjunct therapy, which is also called adjunctive therapy) chemotherapy and/or radiotherapy.

The chemotherapy and/or radiotherapy may he referred to as the primary a tumor or anti-cancer therapy that is being administered to the subject to treat the particular cancer. In some embodiments, a method for reducing (i.e., inhibiting, diminishing) chemosensitivity and/or radiosensitivity of hematopoietic stem cells (HSC) to the chemotherapeutic drug(s) and/or radiotherapy, respectively, is disclosed, the method comprising administering to a subject in need thereof an effective amount of at least one compound of Formula (I) and/or a pharmaceutical composition comprising at least one compound of Formula (I).

In some embodiments, a method for enhancing (i.e., promoting) survival of hematopoietic stem cells is provided, the method comprising administering to a subject in need thereof at least one compound of Formula (I) or a pharmaceutical composition comprising at least one compound of Formula (I).

In some embodiments, a method for decreasing the likelihood of occurrence of metastasis of cancer cells (also called tumor cells herein) in a subject who is in need thereof is disclosed, the method comprising administering an effective amount of at least one compound of Formula (I) and/or a pharmaceutical composition comprising at least one compound of Formula (I).

In some embodiments, a method for treatment and/or prevention of at least one cancer in which the cancer cells may leave the primary site is disclosed, the method comprising administering to a subject in need thereof an effective amount of at least one compound of Formula (I) and/or a pharmaceutical composition comprising at least one compound of Formula (I). A primary site may be, for example, solid tissue (e.g., breast or prostate) or the bloodstream.

In some embodiments, a method for treatment and/or prevention of at least one cancer in which it is desirable to mobilize cancer cells from a site into the bloodstream and/or retain the cancer cells in the bloodstream is disclosed, the method comprising administering to a subject in need thereof an effective amount of at least one compound of Formula (I) and/or a pharmaceutical composition comprising at least one compound of Formula (I).

In some embodiments, a method for decreasing the likelihood of occurrence of infiltration of cancer cells into bone marrow is disclosed, the method comprises administering to a subject in need thereof an effective amount of at least one compound of Formula (I) and/or a pharmaceutical composition comprising at least one compound of Formula (I).

In some embodiments, a method for releasing cells into circulating blood and enhancing retention of the cells in the blood is disclosed, the method comprising administering to a subject in need thereof an effective amount of at least one compound of Formula (I) and/or a pharmaceutical composition comprising at least one compound of Formula (I). In some embodiments, the method further includes collecting the released cells. In some embodiments, collecting the released cells utilizes apheresis. In some embodiments, the released cells are stern cells (e.g., bone marrow progenitor cells). In some embodiments, G-CSF is administered to the individual.

In some embodiments, a method for treating and/or preventing thrombosis is disclosed, the method comprising administering to a subject in need thereof an effective amount of at least one compound of Formula (I) and/or a pharmaceutical composition comprising at least one compound of Formula (I).

In some embodiments, a method for treating and/or preventing mucositis is disclosed, the method comprising administering to a subject in need thereof an effective amount of at least one compound of Formula (I) and/or a pharmaceutical composition comprising at least one compound of Formula (I).

In some embodiments, a compound of Formula (I) and/or a pharmaceutical composition comprising at least one compound of Formula (I) may be used for the preparation and/or manufacture of a medicament for use in treating and/or preventing at least one of the diseases, disorders, and/or conditions described herein.

Whenever a term in the specification is identified as a range (e.g., C₁₋₄ alkyl), the range independently discloses and includes each element of the range. As a non-limiting example, C₁₋₄ alkyl groups includes, independently, C₁ alkyl groups, C₂ alkyl groups, C₃ alkyl groups, and C₄ alkyl groups.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, teributyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group, Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthiylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “E-selectin antagonist” includes inhibitors of E-selectin only, as well as inhibitors of E-selectin and either P-selectin or L-selectin, and inhibitors of E-selectin, P-selectin, and L-selectin.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluomethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quatemized; and the heterocyclyl group Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diaiylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatorn such as oxygen in oxo, carbonyl, carboxyl, and ester groups; and nitrogen in groups such as inclines, oximes, hydrazones, and nitriles.

The present disclosure includes within its scope all the possible geometric isomers, e.g., Z and E isomers (cis and trans isomers), of the compounds as well as all the possible optical isomers, e.g., diastereoiners and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof.

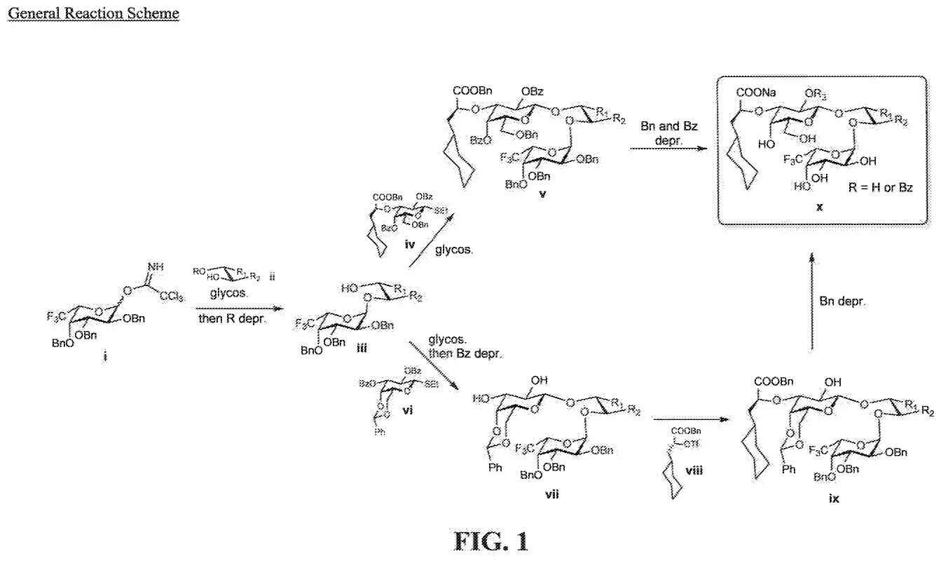

Compounds of Formula (I) may be prepared according to the General Reaction Scheme shown in FIG. 1. It is understood that one of ordinary skill in the art may be able to make these compounds by similar methods or by combining other methods known to one of ordinary skill in the art. It is also understood that one of ordinary skill in the art would be able to make, in a similar manner as described in FIG. 1, other compounds of Formula (I) not specifically illustrated herein by using appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. and/or synthesized according to sources known to those of ordinary skill in the art (see, for example, Advanced Organic Chemistry: Reactions, Mechanisms, and. Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkyisilyl (for example, t-butyldimethylsilyl, t-butyldiphenylsilyl or trimethylsilyl), tetrahydropyranyl, benzyl, and the like. Suitable protecting groups for amino, amidino and guanidine include hut are not limited to t-butoxycarbonyl, benzyloxycarbonyl, and the like. Suitable protecting groups for mercapto include but are not limited to —C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotriryl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John. Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals; Starting Materials and Intermediates; An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell based activity assays.

An inhibition assay may be used to screen for antagonists of E-selectin. For example, an assay may be performed to characterize the capability of a compound described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, E-selectin/Ig chimera may be immobilized onto a matrix (e.g, a multi-well plate, which may be made from a polymer, such as polystyrene; a test tube, and the like); a composition may be added to reduce nonspecific binding (e.g., a composition comprising non-fat dried milk or bovine serum albumin or other blocking buffer routinely used by a person skilled in the art); the immobilized E-selectin may be contacted with the candidate compound in the presence of sLe^{a} comprising a reporter group under conditions and for a time sufficient to permit sLe^{a} to bind to the immobilized E-selectin; the immobilized E-selectin may be washed; and the amount of sLe^{a} bound to immobilized E-selectin may be detected. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing E-selectin antagonists include animal model studies. Non-limiting examples of animal models for liquid cancers used in the art include multiple myeloma (see, e.g., DeWeerdt, Nature 480:S38S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 Apr. 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

The compounds of the present disclosure and the pharmaceutical compositions comprising at least one of such compounds may be useful in methods for treating and/or preventing a disease or disorder that is treatable by inhibiting at least one activity of E-selectin (and/or inhibiting binding of E-selectin to a ligand, which in tum inhibits a biological activity). Focal adhesion of leukocytes to the endothelial lining of blood vessels is a characteristic step in certain vascular disease processes.

The compounds of the present disclosure and pharmaceutical compositions comprising at least one such compound may be useful in methods for treating and/or preventing at least one inflammatory disease. Inflammation comprises reaction of vascularized living tissue to injury. By way of example, although E-selectin-mediated cell adhesion is important to the body's anti-infective immune response, in other circumstances, E-selectin mediated cell adhesion may be undesirable or excessive, resulting in tissue damage instead of repair. For example, many pathologies (such as autoimmune and inflammatory diseases, shock and reperfusion injuries) involve abnormal adhesion of white blood cells. Therefore, inflammation affects blood vessels and adjacent tissues in response to an injury or abnormal stimulation by a physical, chemical, or biological agent. Examples of inflammatory diseases, disorders, or conditions include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the glycomimetic compounds described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischaemic episodes, prevention of restenosis associated with vascular stenting, and for undesirable angiogenesis, for example, angiogenesis associated with tumor growth.

As discussed in detail herein, a disease or disorder to be treated or prevented is a cancer and related metastasis and includes cancers that comprise solid tumor(s) and cancers that comprise liquid tumor(s). The compounds of the present disclosure and pharmaceutical compositions comprising at least one such compound may be useful in methods for preventing and/or treating cancer. In some embodiments, the at least one compound may be used for treating and/or preventing metastasis and/or for inhibiting (slowing, retarding, or preventing) metastasis of cancer cells.

In some embodiments, the compounds of present disclosure and pharmaceutical compositions comprising at least one such compound may be used for decreasing (i.e., reducing) the likelihood of occurrence of metastasis of cancer cells in an individual (i.e., subject, patient) who is in need thereof. The compounds of the present disclosure and compositions comprising at least one such compound may be used for decreasing (i.e., reducing) the likelihood of occurrence of infiltration of cancer cells into bone marrow in an individual who is in need thereof. The individuals (or subjects) in need of such treatments include subjects who have been diagnosed with a cancer, which includes cancers that comprise solid tumor(s) and cancers that comprise liquid tumor(s).

Non-limiting examples of cancers include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors can occur in the blood, bone marrow, the soft, sponge-like tissue in the center of most hones, and lymph nodes and include leukemia (e.g., AML, ALL, CLL, and CML), lymphoma, and myeloma (e.g., multiple myeloma).

Lymphomas include Hodgkin lymphoma, which is marked by the presence of a type of cell called the Reed-Stemberg cell, and non-Hodgkin lymphomas, which includes a large, diverse group of cancers of immune system cells. Non-Hodgkin lymphomas can be further divided into cancers that have an indolent (slow-growing) course and those that have an aggressive (fast-growing) course, and which subtypes respond to treatment differently.

The compounds of the present disclosure and pharmaceutical. compositions comprising at least one such compound may be administered as an adjunct therapy to chemotherapy and/or radiotherapy, which is/are being delivered to the subject as primary therapy for treating the cancer. The chemotherapy and/or radiotherapy that may be administered depend upon several factors including the type of cancer, location of the tumor(s), stage of the cancer, age and gender and general health status of the subject. A person of ordinary skill in the medical art can readily determine the appropriate chemotherapy regimen and/or radiotherapy regimen for the subject in need. The person of ordinary skill in the medical art can also determine, with the aid of preclinical and clinical studies, when the compound of the present disclosure or pharmaceutical composition comprising at least one such compound should be administered to the subject, that is whether the compound or composition is administered prior to, concurrent with, or subsequent to a cycle of the primary chemotherapy or radiation treatment.

Also provided herein is a method for inhibiting adhesion of a tumor cell that expresses a ligand of E-selectin to an endothelial cell expressing E-selectin on its cell surface, which method comprises contacting the endothelial cell with at least one compound of the present disclosure or pharmaceutical compositions comprising at least one such compound, thereby permitting the compound to interact with E-selectin on the endothelial cell surface and inhibiting binding of the tumor cell to the endothelial cell. Without wishing to be bound by theory, inhibiting adhesion of tumor cells to endothelial cells may reduce in a significant manner, the capability of the tumor cells to extravasate into other organs, blood vessels, lymph, or bone marrow and thereby reduce, decrease, or inhibit, or slow the progression of the cancer, including reducing, decreasing, inhibiting, or slowing metastasis.

As described herein, at least one of the compounds of the present disclosure or pharmaceutical compositions comprising at least one such compound may be administered in combination with at least one additional anti-cancer agent. Chemotherapy may comprise one or more chemotherapeutic agents. For example, chemotherapy agents, radiotherapeutic agents, inhibitors of phosphoinositide-3 kinase (PI3K), and inhibitors of VEGF may be used in combination with an E-selectin antagonist compound described herein. Non-limiting examples of inhibitors of PI3K. include the compound named by Exelixis as “XL499.” Non-limiting examples of VEGF inhibitors include the compound called “cabo” (previously known as XL184). Many other chemotherapeutics are small organic molecules. As understood by a person of ordinary skill in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents; antimetabolites; anthracyclines, plant alkaloids; and topoisomerase inhibitors.

The compounds of the present disclosure or pharmaceutical compositions comprising at least one such compound may function independently from the anti-cancer agent or may function in coordination with the anti-cancer agent, e.g., by enhancing effectiveness of the anti-cancer agent or vice versa. Accordingly, provided herein are methods for enhancing (i.e., enhancing, promoting, improving the likelihood of, enhancing in a statistically or biologically significant manner) and/or maintaining survival of hematopoietic stem cells (HSC) in a subject who is treated with and/or will be treated with a chemotherapeutic drug(s) and/or radioactive therapy, respectively, comprising administering at least one E-selectin antagonist glycomimetic compound as described herein. In some embodiments, the subject receives and/or will receive both chemotherapy and radiation therapy. Also, provided herein is a method for reducing (i.e., reducing, inhibiting, diminishing in a statistically or biologically significant manner) chemosensitivity and/or radiosensitivity of hematopoietic stern cells (HSC) to the chemotherapeutic drug(s) and/or radioactive therapy, respectively, in a subject. Because repeated cycles of chemotherapy and radiotherapy often diminish the ability of HSCs to recover and replenish bone marrow, the glycomimetic compounds described herein may be useful for subjects who will receive more than one cycle, such as at least two, three, four or more cycles, of chemotherapy and/or radiotherapy. HSCs reside in the bone marrow and generate the cells that are needed to replenish the immune system and the blood. Anatomically, bone marrow comprises a vascular niche that is adjacent to bone endothelial sinuses (see, e.g., Kiel et al., Cell 121:1109-21 (2005); Sugivama et al., Immunity 25:977-88 (2006); Mendez-Ferrer et al., Nature 466:829-34 (2010); Butler et al., Cell Stem Cell 6:251-64 (2010)). A recent study describes that E-selectin promotes HSC proliferation and is an important component of the vascular niche (see, e.g., Winkler et al., Nature Medicine published online 21 Oct. 2012; doi:10.1038/nm.2969). Deletion or inhibition of E-selectin enhanced HSC survival in mice that were treated with chemotherapeutic agents or radiotherapy and accelerated blood neutrophil recovery (see, e.g., Winkler et al., supra).

In addition, the administration of at least one compound of the present disclosure or pharmaceutical composition comprising at least one such compounds may be in conjunction with one or more other therapies, e.g., for reducing toxicities of therapy. For example, at least one palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one E-selectin antagonist described herein may be administered before, after, or concurrently with administration of at least one additional anti-cancer agent or at least one palliative agent to reduce a side effect of therapy. When administration is concurrent, the combination may be administered from a single container or two (or more) separate containers.

Cancer cells (also called herein tumor cells) that may be prevented (i.e., inhibited, slowed) from metastasizing, from adhering to an endothelial cell, or from infiltrating bone marrow include cells of solid tumors and liquid tumors (including hematological malignancies). Examples of solid tumors are described herein and include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, and lymph nodes and include leukemia (e.g, AML, ALL, CLL, and CML), lymphoma (e.g., Hodgkin lymphoma and non-Hodgkin lymphoma), and myeloma (e.g., multiple myeloma). As used herein, the term cancer cells include mature, progenitor, and cancer stem cells.

Bones are a common location for cancer to infiltrate once leaving the primary tumor location. Once cancer resides in bone, it is frequently a cause of pain to the individual. In addition, if the particular bone affected is a source for production of blood cells in the bone marrow, the individual may develop a variety of blood cell related disorders. Breast and prostate cancer are examples of solid tumors that migrate to bones. Acute myelogenous leukemia (AML) and multiple myeloma (MM) are examples of liquid tumors that migrate to bones. Cancer cells that migrate to bone will typically migrate to the endosteal region of the bone marrow. Once cancer cells have infiltrated into the marrow, the cells become quiescent and are protected from chemotherapy. The compounds of the present disclosure block infiltration of disseminated cancer cells into bone marrow. A variety of individuals may benefit from treatment with the compounds. Examples of such individuals include individuals with a cancer type having a propensity to migrate to bone where the tumor is still localized or the tumor is disseminated but not yet infiltrated bone, or where individuals with such a cancer type are in remission.

The cancer patient population most likely to respond to treatment using the E-selectin antagonist agents (e.g., compounds of Formula (I)) described herein can be identified based on the mechanism of action of E-selectin. That is, patients may be selected that express a highly active E-selectin as determined by the genetic polymorphism for E-selectin of S128R (Alessandro et al., Int. J. Cancer 121:528-535, 2007). In addition, patients for treatment by the compounds described herein may also selected based on elevated expression of the E-selectin binding ligands (sialyl Le^{a} and sialyl Le^{x}) as determined by antibodies directed against cancer-associated antigens CA-19-9 (Zheng et al., World J. Gastroenterol. 7:431-434, 2001) and CD65. In addition, antibodies HECA-452 and FH-6 which recognize similar carbohydrate ligands of E-selectin may also be used in a diagnostic assay to select the cancer patient population most likely to respond to this treatment.

The compounds of the present disclosure and pharmaceutical compositions comprising at least one such compound may be useful in methods for treating and/or preventing thrombosis. As described herein methods are provided for inhibiting formation of a thrombus or inhibiting the rate at which a thrombus is formed. These methods may therefore be used for preventing thrombosis (i.e., reducing or decreasing the likelihood of occurrence of a thrombus in a statistically or clinically significant manner).

Thrombus formation may occur in infants, children, teenagers and adults. An individual may have a hereditary predisposition to thrombosis. Thrombosis may be initiated, for example, due to a medical condition (such as cancer or pregnancy), a medical procedure (such as surgery) or an environmental condition (such as prolonged immobility). Other individuals at risk for thrombus formation include those who have previously presented with a thrombus.

The compounds of the present disclosure and pharmaceutical compositions comprising at least one such compound may be useful in methods for treating individuals undergoing thrombosis or who are at risk of a thrombotic event occurring. Such individuals may or may not have a risk of bleeding. In some embodiments, the individual has a risk of bleeding. In some embodiments, the thrombosis is a venous thromboembolism (VTE). VTE causes deep vein thrombosis and pulmonary embolism. Low molecular weight (LMW) heparin is the current mainstay therapy for the prevention and treatment of VTE. In many circumstances, however, the use of LAM heparin is contraindicated. LMW heparin is a known anti-coagulant and delays clotting over four times longer than control bleeding times. Patients undergoing surgery, patients with thrombocytopenia, patients with a history of stroke, and many cancer patients should avoid administration of heparin due to the risk of bleeding. By contract, administration of the E-selectin antagonist compounds of Formula (I) significantly reduces the time to clotting than occurs when LMW heparin is administered, and thus provide a significant improvement in reducing bleeding time compared with LMW heparin. Accordingly, the compounds and pharmaceutical compositions described herein may not only be useful for treating a patient for whom the risk of bleeding is not significant, but also may be useful in when the risk of bleeding is significant and the use of anti-thrombosis agents with anti-coagulant properties (such as LMW heparin) is contraindicated.

The compounds of the present disclosure and pharmaceutical compositions comprising at least one such compound may be administered in combination with at least one additional anti-thrombosis agent. The compounds of the present disclosure and pharmaceutical compositions comprising at least one such compound may function independently from the anti-thrombosis agent or may function in coordination with the at least one anti-thrombosis agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery and/or counteract side effects of administration of antibiotics or corticosteroids are examples of such palliative agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

The compounds of the present disclosure and pharmaceutical compositions comprising at least one such compound may be useful in methods for preventing and/or treating mucositis. In some embodiments, at least one compound of Formula (I) or a pharmaceutical composition comprising at least one compound of Formula (I) may be used in methods described herein for decreasing the likelihood of occurrence of mucositis in a subject who is in need thereof by administering the compound or composition to the subject. In some embodiments, the mucositis is chosen from oral mucositis, esophageal mucositis, and gastrointestinal mucositis. In some embodiments, the mucositis is alimentary mucositis.

It is believed that approximately half of all cancer patients undergoing therapy suffer some degree of mucositis. Mucositis is believed to occur, for example, in virtually all patients treated with radiation therapy for head and neck tumors, all patients receiving radiation along the GI tract, and approximately 40% of those subjected to radiation therapy and/or chemotherapy for tumors in other locations (e.g., leukemias or lymphomas). It is also is believed to be highly prevalent in patients treated with high dose chemotherapy and/or irradiation for the purpose of myeloablation, such as in preparation for stem cell or bone marrow transplantation. The compounds of the present disclosure and pharmaceutical compositions comprising at least one such compound may be useful in methods for treating and/or preventing mucositis in a subject afflicted with cancer. In some embodiments, the subject is afflicted with a cancer chosen from head and neck cancer, breast cancer, lung cancer, ovarian cancer, prostate cancer, lymphatic cancer, leukemic cancer, and/or gastrointestinal cancer. In some embodiments, the mucositis is associated with radiation therapy and/or chemotherapy. In some embodiments, the chemotherapy comprises administering a therapeutically effective amount of at least one compound chosen from platinum, cisplatin, carboplatin, oxaliplatin, mechlorethamine, cyclophosphamide, chlorambucil, azathioprine, mercaptopurine, vincristine, vinblastine, vinorelbine, vindesine, etoposide, teniposide, paclitaxel, docetaxel, irinotecan, topotecan, arnsacrine, etoposide, etoposide phosphate, teniposide, 5-fluorouracil (5-FU), leucovorin, methotrexate, gemcitabine, taxane, leucovorin, mitomycin C, tegafur-uracil, idarubicin ,fludarabine, mitoxantrone, ifosfamide and doxoruhicin.

In some embodiments, the method further comprises administering a therapeutically effective amount of at least one MMP inhibitor, inflammatory cytokine inhibitor, mast cell inhibitor, NSAID, NO inhibitor, or antimicrobial compound.

In some embodiments, the method further comprises administering a therapeutically effective amount of velaferrnin and/or palifermin.

The compounds of the present disclosure and pharmaceutical compositions comprising at least one such compound may he useful in methods for mobilizing cells from the bone marrow to the peripheral vasculature and tissues. As discussed herein, in some embodiments, the compounds and compositions are useful for mobilizing hematopoietic cells, including hematopoietic stem cells and hematopoietic progenitor cells. In some embodiments, the compounds act as mobilizing agents of normal blood cell types. In some embodiments, the agents are used in methods for mobilizing mature white blood cells (which may also be called leukocytes herein), such as granulocytes (e.g., neutrophils, eosinophils, basophils), lymphocytes, and monocytes from the hone marrow or other immune cell compartments such as the spleen and liver. Methods are also provided for using the compounds of the present disclosure and pharmaceutical compositions comprising at least one such compound in methods for mobilizing tumor cells from the bone marrow. The tumor cells may be malignant cells (e.g., tumor cells that are metastatic cancer cells, or highly invasive tumor cells) in cancers. These tumor cells may be of hematopoietic origin or may be malignant cells of another origin residing in the bone.

In some embodiments, the methods using the E-selectin antagonists described herein are useful for mobilizing hematopoietic cells, such as hematopoietic stem cells and progenitor cells and leukocytes (including granulocytes such as neutrophils), which are collected (i.e., harvested, obtained) from the subject receiving the E-selectin antagonist and at a later time are administered back into the same subject (autologous donor) or administered to a different subject (allogeneic donor). Hematopoietic stem cell replacement and hematopoietic stem cell transplantation have been successfully used for treating a number of diseases (including cancers) as described herein and in the art. By way of example, stem cell replacement therapy or transplantation follows myeloablation of a subject, such as occurs with administration of high dose chemotherapy and/or radiotherapy. Desirably, an allogeneic donor shares sufficient HLA antigens with the recipient/subject to minimize the risk of host versus graft disease in the recipient (i.e., the subject receiving the hematopoietic stem cell transplant). Obtaining the hematopoietic cells from the donor subject (autologous or allogeneic) is performed by apheresis or leukapheresis. HLA typing of a potential donor and the recipient and apheresis or leukapheresis are methods routinely practiced in the clinical art.

By way of non-limiting example, autologous or allogenic hematopoietic stem cells and progenitors cells may be used for treating a recipient subject who has certain cancers, such as Hodgkin lymphoma, non-Hodgkin lymphoma, or multiple myeloma. Allogeneic hematopoietic stem cells and progenitors cells may be used, for example, for treating a recipient subject who has acute leukemias (e.g., AML, ALL); chronic lymphocytic leukemia (CLL); amegakaryocytosis/congenital thrombocytopenia; aplastic anemia/refractory anemia; familial erythrophagocytic lymphohistiocytosis; myelodysplastic syndrome/other myelodysplastic disorders; osteopetrosis; paroxysmal nocturnal hemoglobinuria; and Wiskott-aldrich syndrome, for example. Exemplary uses for autologous hematopoietic stem cells and progenitors cells include treating a recipient subject who has amyloidosis; germ cell tumors (e.g., testicular cancer); or a solid tumor. Allogeneic hematopoietic stem cell transplants have also been investigated for use in treating solid tumors (see, e.g., Ueno et al., Blood 102:3829-36 (2003)).

In some embodiments of the methods described herein, the subject is not a donor of peripheral hematopoietic cells but has a disease, disorder, or condition for which mobilization of hematopoietic cells in the subject will provide clinical benefit. Stated another way, while this clinical situation is similar to autologous hematopoietic cell replacement, the mobilized hematopoeitic cells are not removed and given back to the same subject at a later time as occurs, for example, with a subject who receives myeloablation therapy. Accordingly, methods are provided for mobilizing hematopoietic cells, such as hematopoietic stem cells and progenitor cells and leukocytes (including granulocytes, such as neutrophils), by administering at least once compound of Formula (I). Mobilizing hematopoietic stem cells and progenitor cells may be useful for treating an inflammatory condition or for tissue repair or wound healing. See, e.g., Mimeault et al., Clin. Pharmacol. Therapeutics 82:252-64 (2007).

In some embodiments, the methods described herein are useful for mobilizing hematopoietic leukocytes (white blood cells) in a subject, which methods may be used in treating diseases, disorders, and conditions for which an increase in white blood cells, such as neutrophils, eosinophils, lymphocytes, monocytes, basophils, will provide clinical benefit. For example, for cancer patients, the compounds of Formula (I) are beneficial for stimulating neutrophil production to compensate for hematopoietic deficits resulting from chemotherapy or radiation therapy. Other diseases, disorders, and conditions to be treated include infectious diseases and related conditions, such as sepsis. When the subject to whom at least one compound of Formula (I) is administered is a donor, neutrophils may be collected for administration to a recipient subject who has reduced hematopoietic, function, reduced immune function, reduced neutrophil count, reduced neutrophil mobilization, severe chronic neutropenia, leucopenia, thrombocytopenia, anemia, and acquired immune deficiency syndrome. Mobilization of mature white blood cells may be useful in subjects to improve or to enhance tissue repair, and to minimize or prevent vascular injury and tissue damage, for example following liver transplantation, myocardial infarction or limb ischemia. See, e.g., Pelus, Curr. Opin. Hematol. 15:285-92 (2008); Lemoli et al., Haematologica 93:321-24 (2008).

The compound of Formula (I) may be used in combination with one or more other agents that mobilize hematopoietic cells. Such agents include, for example, G-CSF; AMD3100 or other CXCR4 antagonists; GRO-β (CXCL2) and an N-terminal 4-amino truncated form (SB-251353); IL-8SDF-1α peptide analogs, CTCE-0021 and CTCE-0214; and the SDF1 analog, Met-SDF-1β (see, e.g., Pelus, supra and references cited therein). In some embodiments, a compound of Formula (I) may be administered with other mobilizing agents used in the art, which may peiluit administration of a lower dose of GCSF or AMD3100, for example, than required in the absence of a compound of Formula (I). The appropriate therapeutic regimen for administering a compound of Formula (I) in combination with another mobilizing agent or agents can be readily determined by a person skilled in the clinical art.

The terms, “treat” and “treatment,” include medical management of a disease, disorder, and/or condition of a subject (i.e., patient, individual) as would be understood by a person of ordinary skill in the art (see, e.g., Stedman's Medical Dictionary). In general, an appropriate dose and treatment regimen provide at least one of the compounds of the present disclosure in an amount sufficient to provide therapeutic and/or prophylactic benefit. For both. therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

In some embodiments of the methods described herein, the subject is a human. In some embodiments of the methods described herein, the subject is a non-human animal. Non-human animals that may be treated include mammals, for example, non-human primates (e.g., monkey, chimpanzee, gorilla, and the like), rodents (e.g, rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing diseases, disorders, and/or conditions treatable by inhibiting an activity of E-selectin can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (I). In some embodiments, the pharmaceutical composition further comprises at least one additional pharmaceutically acceptable ingredient.

In pharmaceutical dosage forms, any one or more of the compounds of the present disclosure may be administered in the form of a pharmaceutically acceptable derivative, such as a salt, and/or it or they may also be used alone and/or in appropriate association, as well as in combination, with other pharmaceutically active compounds.

An effective amount or therapeutically effective amount refers to an amount of at least one compound of the present disclosure or a pharmaceutical composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject. In general, the amount of at least one compound of Formula (I) as described herein, that is present in a dose, may range from about 0.01 μg to about 3000 μg per kg weight of the subject. The minimum dose that is sufficient to provide effective therapy may be used in some embodiments. Subjects may generally be monitored for therapeutic effectiveness using assays suitable for the disease, disorder and/or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g, serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical compositions described herein may, for example, be sterile aqueous or sterile non-aqueous solutions, suspensions, or emulsions, and may additionally comprise at least one pharmaceutically acceptable excipient (i.e., a non-toxic material that does not interfere with the activity of the active ingredient). Such compositions may, for example, be in the form of a solid, liquid, or gas (aerosol). Alternatively, the compositions described herein may, for example, be formulated as a lyophilizate, or compounds described herein may be encapsulated within liposomes using technology known in the art. The pharmaceutical compositions may further comprise at least one additional pharmaceutically acceptable ingredient, which may be biologically active or inactive. Non-limiting examples of such ingredients include buffers e. neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g, EDTA and glutathione), stabilizers, dyes, flavoring agents, suspending agents, and preservatives.

Any suitable excipient or carrier known to those of ordinary skill in the art for use in compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient may be selected based on the mode of administration, as well as the chemical composition of the active ingredient(s). Compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned ingredients, excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid composition may include, for example, at least one the following: a sterile diluent such as water for injection, saline solution, including for example physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition is an injectable composition, and in some embodiments, the injectable composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g, in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulatedsustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; the formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The pharmaceutical compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacal. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound of Formula (I) and/or pharmaceutical composition comprising the same.

### EXAMPLES

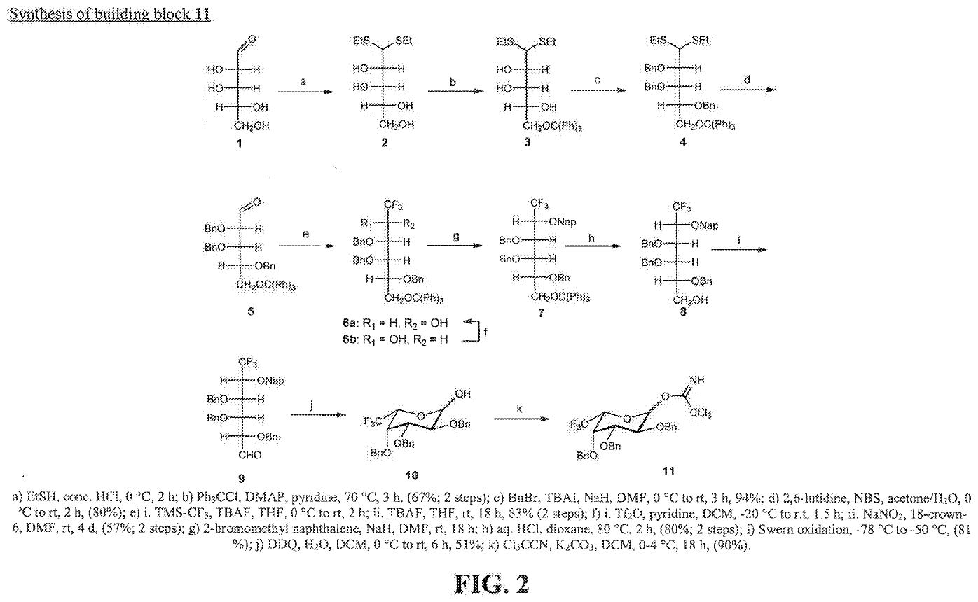

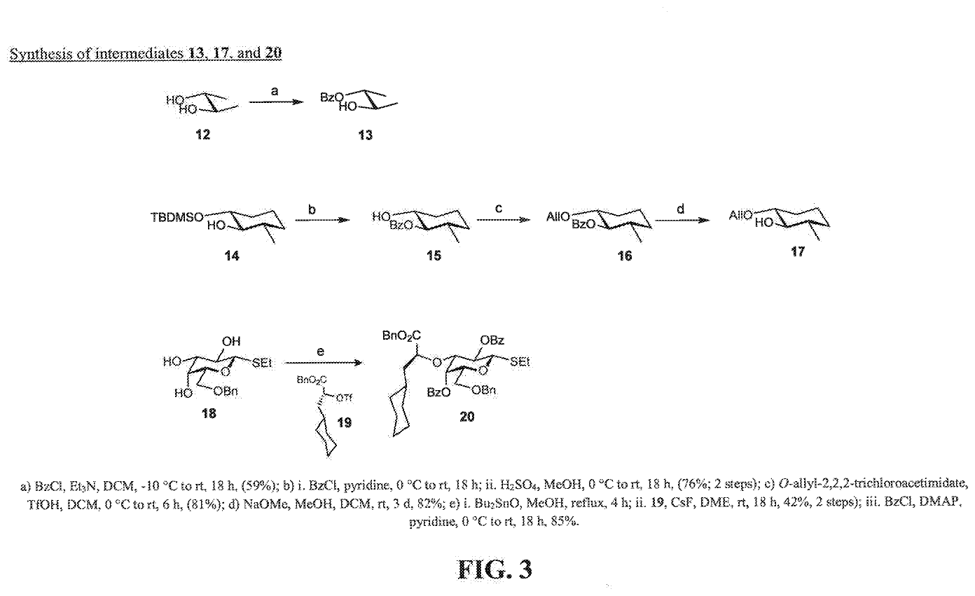

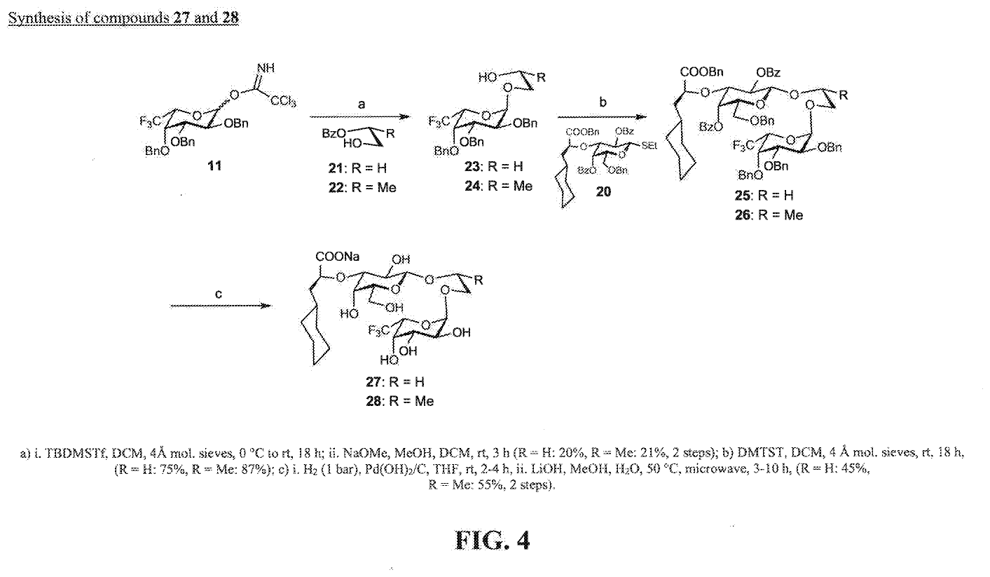

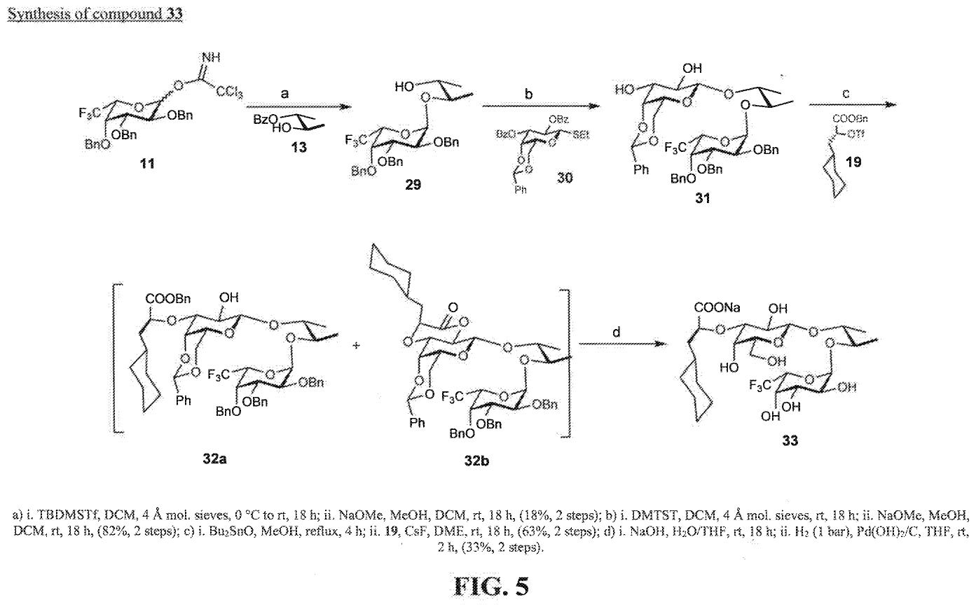

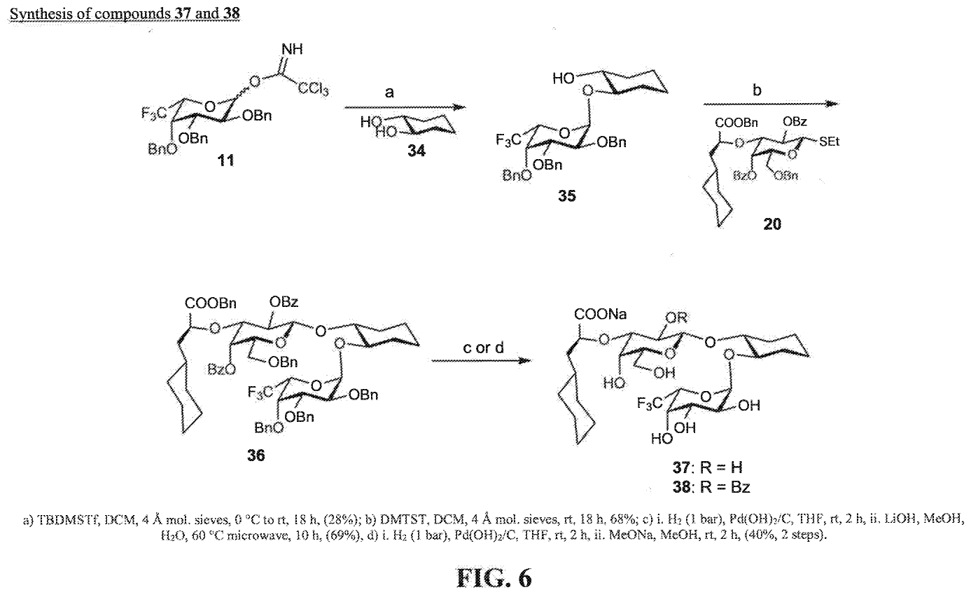

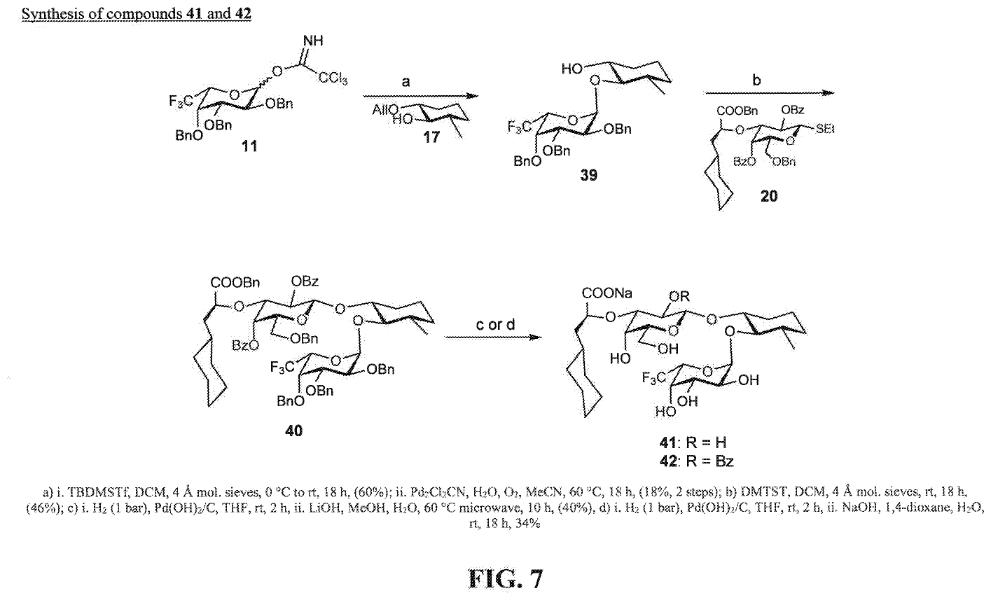

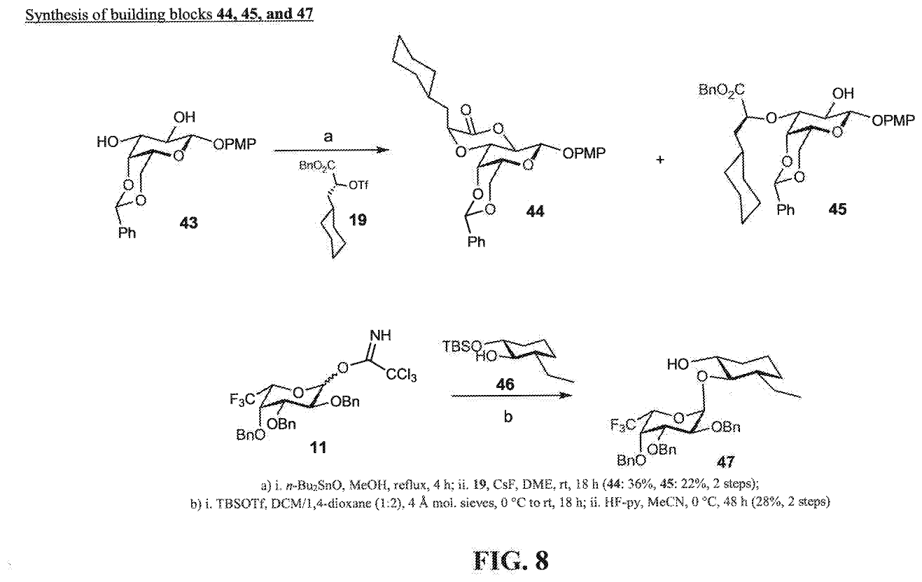

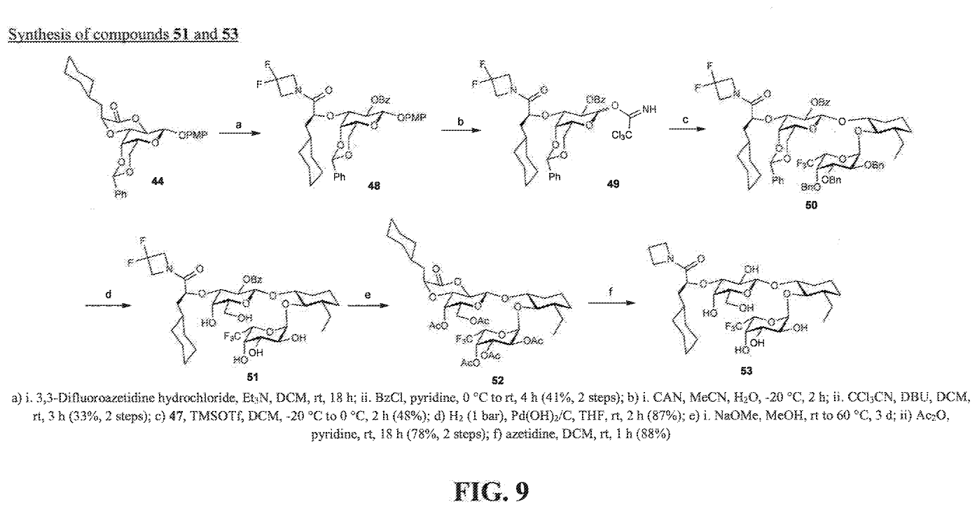

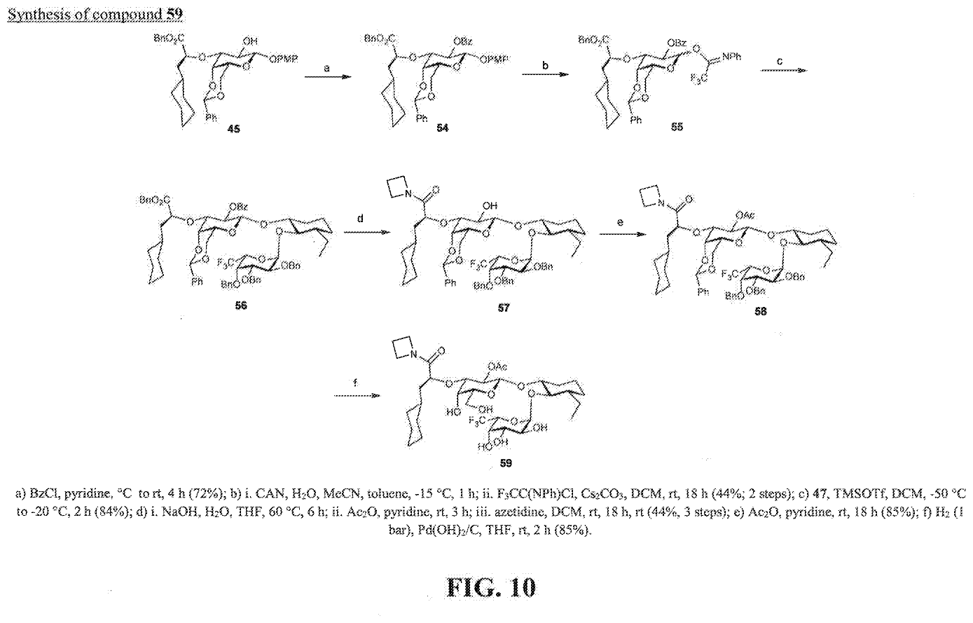

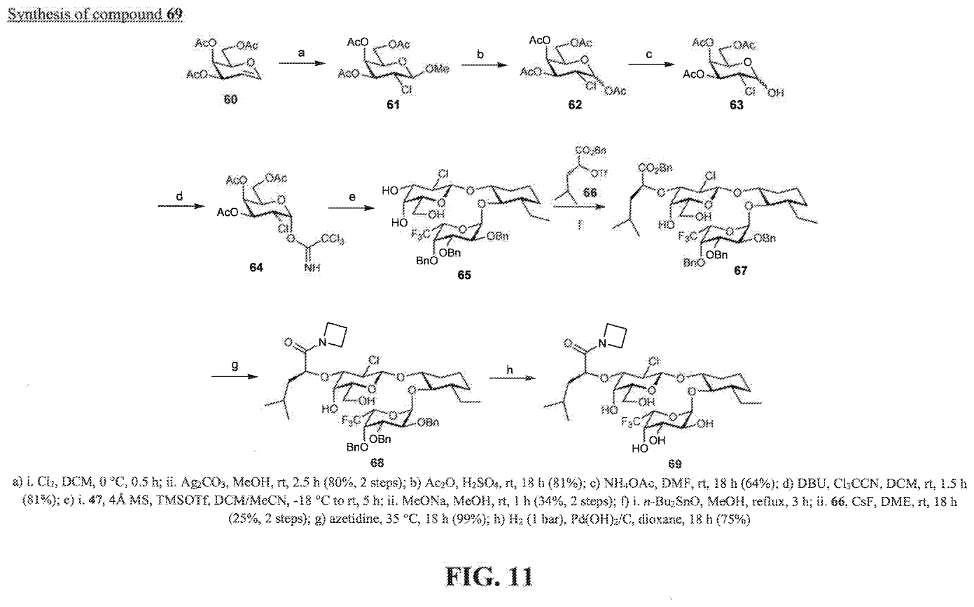

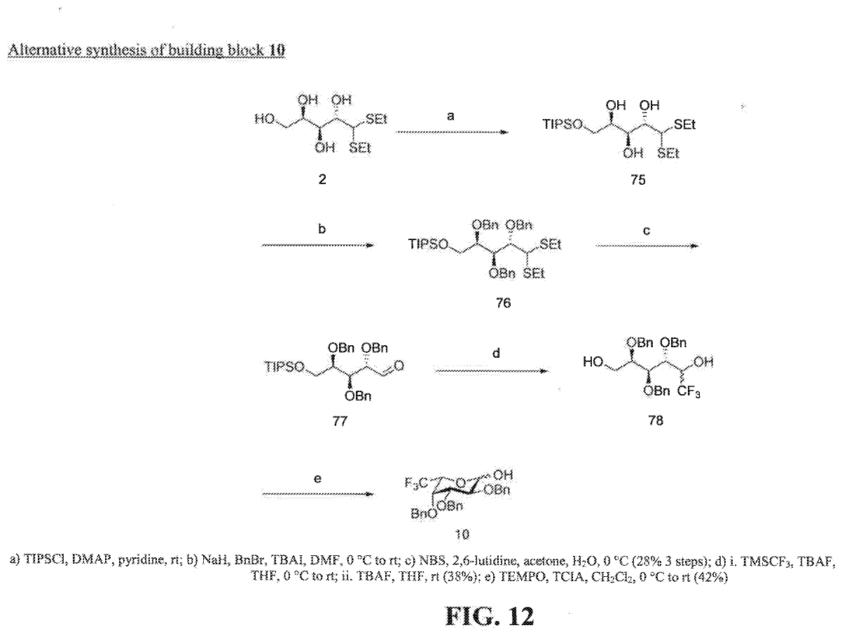

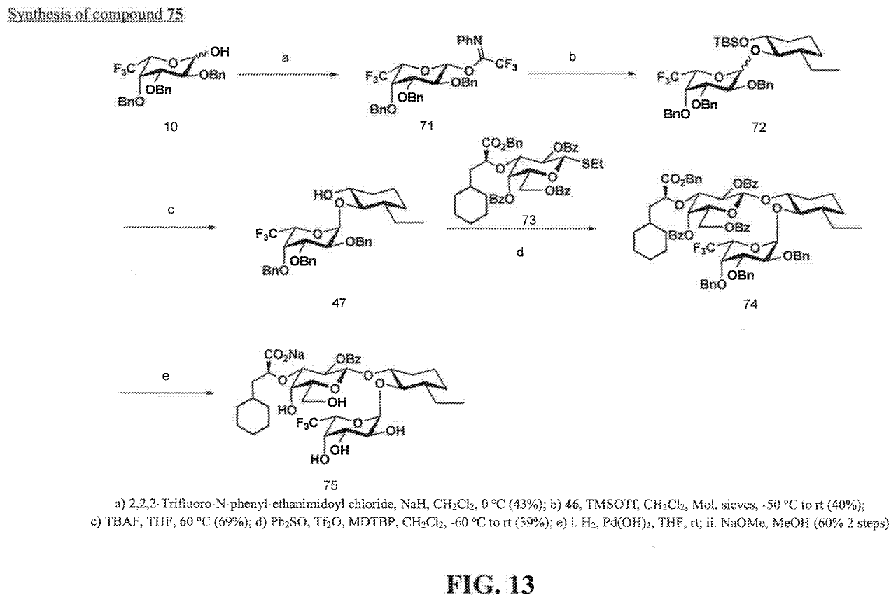

Exemplary glycomimetic E-selectin antagonists of Formula (I) and salts of compounds of Formula (I) were synthesized as described in Examples 1-12 and as shown in the exemplary synthesis schemes set forth in FIGS. 2-13.

### Example 1

### Synthesis of Compound 27

Synthesis of compound 2; To a solution of D-lyxose compound 1 (10.0 g, 66.6 mmol) in concentrated HCl (10 mL) was added thioethanol (13 mL) at 0° C. The reaction was stirred for 2 hours at 0° C. then the mixture was extracted with EtOAc (4×150 mL), the combined organic phases were neutralized by careful addition of solid NaHCO₃, dried over NaSO₄, filtered and concentrated in vacua to give compound 2 (14.3 g, 84%). ¹H NMR (500 MHz, CD₃OD); δ=4.27 (d, J=1.9 Hz, 1H, H-1), 4.00 (dd, J=1.9, 9.1 Hz, 1H, H-2), 3.93 (td, J=1.5, 6.3 Hz, 1H, H-4), 3.78 (dd, J=1.4, 9.1 Hz, 1H, H-3), 3.63 (d, J=6.3 Hz, 2H, H-5), 2.79-2.66 (m, 4H, S—CH₂—CH₃), 3.93 (td, J=6.0, 7.4 Hz, 6H, S—CH₂—CH₃); ¹³C NMR (126 MHz, CDCl₃): δ=75.3 (C-2), 72.2 (C-3), 71.6 (C-4), 65.1 (C-5), 56.0 (C-1), 26.5, 26.3 (S—CH₂—CH₃), 15.1 (S—CH₂—CH₃).

Synthesis of compound 2: To a solution of the crude product compound 2 (14.3 g) obtained in the previous step and DMAP (0.68 g, 5.6 mmol) in anhydrous pyridine (100 mL) was added TrCl (20.3 g, 72.9 mmol) in portions, and the resulting mixture was heated to 70° C. for 3 hours. The reaction was allowed to cool down to room temperature, and the solvent was removed in vacuo. The crude product was dissolved in DCM (1 L), washed with saturated aqueous NH₄Cl and saturated aqueous NaHCO₃, dried over Na₂SO₄, filtered, and concentrated in vacuo. The crude residue was purified by flash chromatography (petroleum ether/EtOAc, gradient 0-40%) to give compound 3 (22.3 g, 67%, 2 steps). [α]_{D}²²++10.3 (c 0.45, CHCl₃); NMR (500 MHz, CDCl₃): δ=7.45-7.41 (m, 5H, Ar—H), 7.34-7.28 (m, 6H, Ar—H), 7.27-7.22 (m, 4H, Ar—H), 4.17 (d, J=3.6 Hz, 1H, H-1), 4.13 (m, 1H, H-4), 3.90 (m, 1H, H-2), 3.83 (m, 1H, H-3), 3.43 (dd, J=4.5, 9.6 Hz, 1H, H-5a), 3.32 (dd, J=6.1, 9.6 Hz, 1H, H-5b), 2.82-2.73 (m, 3H, C—OH), 2.71 (q, J=7.5 Hz, 2H, S—CH₂—CH₃), 2.66 (q, J=7.4 Hz, 2H, Hz, S—CH₂—CH₃), 1.30-1.23 (m, 6H, S—CH₂—CH₃); ¹³C NMR (126 MHz, CDCl₃): δ=143.6, 128.6, 128.0, 127.2 (Ar—C), 87.2 (Ph₃C), 73.2 (C-2), 71.9 (C-3), 69.1 (C-4), 66.2 (C-5), 54.9 (C-1), 25.6, 25.6 (S—CH₂—CH₃), 14.7, 14.5 (S—CH₂—CH₃); ESI-MS: m/z: Calculated for C₂₈H₃₄NaO₄S₂ [M+Na]⁺: 521.2, found: 521.1.

Synthesis of compound 4: To a solution of compound 3 (22.0 g, 44.1 mmol) and TBAI (1.62 g, 4.41 mmol) in dry DMF (100 mL) NaH (60% in oil, 7.0 g, 176 mmol) was added in portions over 30 minutes at 0° C. The resulting suspension was stirred for 30 minutes at 0° C., then BnBr (18.8 mL, 160 mmol) was added dropwise. The reaction was stirred at room temperature for 3 hours, diluted with petroleum ether/Et₂O (1:1, 100 mL) and quenched by careful addition of water (50 mL), The water phase was extracted with petroleum ether/Et₂O (1:1, 2×200 mL), the combined organic phases were washed with water (2×50 mL) and brine (50 mL), dried over Na₂SO₄, filtered, and concentrated in vacuo. The snide product was purified by flash chromatography (petroleum ether/EtOAc, gradient 0-20%) to yield compound 4 (32.0 g, 94%) as colorless wax, [α]_{D}²² −5.2 (c 0.45, CHCl₃); NMR (500 MHz, CDCl₃): δ=7.44-7.38 (m, 6H, Ar—H), 7.32-7.18 (m, 22H, Ar—H), 7.16-7.10 (m, 2H, Ar—H), 4.97 (A of AB, J=11.3 Hz, 1H, PhCH₂), 4.61 (A′ of A′B′, J=11.8 Hz, 1H, PhCH₂), 4.60 (A″ of A″B″, J=11.4 Hz, 1H, PhCH₂), 4.50 (B″ of A″B″, J=11.5 Hz, 1H, PhCH₂), 4.45 (B′ of A′B′, J=11.8 Hz, 1H, PhCH₂), 4.40 (B of AB, J=11.3 Hz, 1H, PhCH₂), 4.19 (d, J=2.3 Hz, 1H, H-1), 4.13 (dd, J=2.4, 8.0 Hz, 1H, H-3), 4.08 (dd, J=2.3, 8.0 Hz, 1H, H-2), 3.43 (td, J=2.3, 6.2 Hz, 1H, H-4), 3.46 (dd, J=6.1, 9.7 Hz, 1H, H-5a), 3.28 (dd, J=6.4, 9.7 Hz, 1H, H-5b), 2.72-2.50 (m, 4H, S—CH₂—CH₃), 1.22-1.16 (m, 6H, S—CH₂—CH₃); ¹³C NMR (126 MHz, CDCl₃): δ=143.9, 138.8, 138.7, 138.4, 128.7, 128,2, 128.2, 128.2, 128.0, 127.8, 127.8, 127.5, 127.5, 127.4, 127.3, 127.0 (Ar—C), 87.1 (Ph₃C), 82.4 (C-2), 79.5 (C-3), 78.0 (C-4), 74.5, 74.4, 72.8 (3 PhCH₂), 63.5 (C-5), 53.8 (C-1), 26.6, 25.1 (S—CH₂—CH₃), 14.6, 14.4 (S—CH₂—CH₃); ESI-MS: m/z: Calculated for C₄₉H₅₂NaO₄S₂ [M+Na]⁺: 791.3, found: 791.4.

Synthesis of compounds 5: To a solution of 4 (32.0 g, 41.6 mmol) and 2,6-lutidine (57 mL, 499 mmol) in acetone/water (5:1, 240 mL) NBS (37 g, 208 mmol) was added in portions at 0° C., The reaction was stirred at room temperature for 2 h, then quenched by addition of saturated aqueous NaHCO₃ (30 mL) and saturated aqueous Na₂S₂O₃ (50 mL) and stirred at room temperature for 30 minutes. The acetone was removed in vacuo and the residual water phase was extracted with Et₂O (3×200 mL), the combined organic phases were washed with 0.05% aqueous HCl (50 mL), saturated aqueous NaHCO3 and brine (50 mL), dried over Na₂SO₄ filtered, and concentrated. The crude product was co-evaporated with xylene (3×100 mL) to remove the excess of 2,6-lutidine. The residue was purified by flash chromatography (petroleum ether/EtOAc, gradient 0-30%) to give 5 (22.0 g, 80%) as colorless wax, [α]_{D}²² −1.6 (c 1.7, CHCl₃); ¹H NMR (500 MHz, CDCl₃): δ=9.58 (d, J=1.5 Hz, 1H, H-1), 7.41-7.36 (m, 6H, Ar—H), 7.32-7.20 (m, 20H, Ar—H), 7.19-7.14 (m, 4H, Ar—H), 4.60 (A of AB, A′ of A′B′, J=11.7 Hz, 2H, PhCH₂), 4.53 (B of AB, J=11.5 Hz, 1H, PhCH₂), 4.52 (A″ of A″B″, J=11.9 Hz, 1H, PhCH₂), 4.47 (B′ of A′B′, J=11.7 Hz, 1H, PhCH₂), 4.35 (B″ of A″B″, J=11.9 Hz, 1H, PhCH₂), 4.08 (m, 1H, H-3), 3.92 (dd, J=1.5, 3.7 Hz, 1H, H-2), 3.80 (dd, J=5.0, 9.9 Hz, 1H, H-4), 3.44 (dd, J=4,4, 10.3 Hz, 1H, H-5a), 3.39 (dd, J=5.5, 10.3 Hz, 1H, H-5b); ¹³C NMR (126 MHz, CDCl₃): δ=201.9 (C-1), 143.9, 138.1, 137.8, 137.4, 129.1, 128.7, 128.4, 128.4, 128.3, 128.2, 128.1, 128.1, 127.9, 127.8, 127.7, 127.0 (Ar—C), 87.0 (Ph₃C), 83.6 (C-2), 80.2 (C-3), 78.6 (C-4), 73.8, 73.0, 72.6 (3 PhCH₂), 63.4 (C-5); ESI-MS: m/z: Calculated for C₄₅H₄₂NaO₅ [M+Na]⁺: 685.3, found: 685.3.

Synthesis of compound 6and 6b: To a solution of compound 5 (21.0 g, 31.6 mmol) in THF (60 mL), CF₃TMS (2M solution in THF, 31.6 mL, 63.3 mmol) was added, followed by TBAF (1 M solution in THF, 1.58 mL, 1.58 mmol). The reaction mixture was stirred for 2 hours, then additional TBAF (1 M solution in THF, 63 mL, 63 mmol) was added. The reaction mixture was stirred for 18 hours, then diluted with EtOAc, washed with brine, dried over Na₂SO₄ and concentrated in vacuo. The crude mixture of diastereoisomers 6a and 6b was purified by flash chromatography (petroleum ether/EtOAc, gradient 0-20%) to isolate the desired product compound 6a (11.3 g, 49%, 2 steps) and the side product compound 6b (8.0 g, 34%, 2 steps), both as colorless wax (note: 6a has higher Rf value than compound 6b; TLC: petroleum ether/EtOAc, 7:3).

Compound 6a: [α]_{D}²² −18.4 (c 1.0, CHCl₃); ¹H NMR (500 MHz, CDCl₃): δ=7.43-7.39 (m, 5H, Ar—H), 7.34-7.20 (m, 23H, Ar—H), 7.16-7.13 (m, 2H, Ar—H), 4.63 (A of AB, J==11.1 Hz, 1H, PhCH₂), 4.62 (A′ of A′B′, J=11.9 Hz, 1H, PhCH₂), 4.53 (B of AB, J=11.1 Hz, 1H, PhCH₂), 4.44 (A″ of A″B″, J=11.3 Hz, 1H, PhCH₂), 4.42 (B′ of A′B′, J=12.0 Hz, 1H, PhCH₂), 4.25 (B″ of A″B″, J=11.0 Hz, 1H, PhCH₂), 4.12 (m, 1H, H-5), 3.96-3.91 (m, 2H, H-3, H-4), 3.75 (m, 1H, H-2), 3.39 (ddd, J=5.7, 10.0, 16.3 Hz, 2H, H-1), 3.32 (d, J=9.3 Hz, 1H, OH); ¹³C NMR (126 MHz, CDCl₃): δ=143.7, 138.1, 137.6, 137.3, 128.6, 128.4, 128.4, 128.3, 128.2, 127.9, 127.9 127.8, 127.3, 127.2 (Ar—C, CF₃), 87.3 (Ph₃C), 78.5 (C-4), 77.3 (C-2), 75.1 (PhCH₂), 74.8, (C-3), 73.5, 72.2 (3C, 3 PhCH₂), 69.1-68.2 (m, C-5), 62.4 (C-1); ¹⁹F NMR (470 MHz, CDCl₃): δ=−76.5 (d, J=7.7 Hz, 3F, CF₃); ESI-MS: m/z: Calculated for C₄₆H₄₃F₃NaO₅ [M+Na]⁺: 755.3, found: 755.3.

Compound 6b: ¹H NMR (500 MHz, CDCl₃): δ=7.43-7.38 (m, 5H, Ar—H), 7.37-7.18 (m, 23H, Ar—H), 7.12-7.07 (m, 2H, Ar—H), 4.72 (A of AB, J=11.3 Hz, 1H, PhCH₂), 4.71 (A′ of A′B′, J =11.5 Hz, 1H, PhCH₂), 4.67 (B of AB, J=11.3 Hz, 1H, PhCH₂), 4.58 (B″ of A′B′, J=11.5 Hz, 1H, PhCH₂), 4.47 (A″ of A″B″, J=11.0 Hz, 1H, PhCH₂), 4.36 (B″ of A″B″, J=11.01 Hz, 1H, PhCH₂), 4.17 (dd, J=1.5, 5.6 Hz, 1H, H-3), 4.06 (m, 1H, H-5), 3.87 (d, J=4.7 Hz, 1H, H-1), 3.82 (m, 1H, H-2), 3.74 (dd, J=1.7, 8.2 Hz, 1H, H-4), 3.57 (dd, J=3.6, 10.8 Hz, 1H, H-1), 3.24 (dd, J=4.8, 10.8 Hz, 1H, H-1′); ¹³C NMR (126 MHz, CDCl₃): δ=143.7, 137.8, 137.7, 137.5, 128.6, 128.5, 128.4, 128.3, 128.0, 127.9, 127.8 127.8, 127.6, 127.1 (Ar—C, CF₃), 87.2 (Ph₃C), 80.4 (C-3), 79.4 (C-2), 78.5, (C-4), 74.6, 73.3, 73.1 (3 PhCH₂), 70.9-69.3 (m, C-5), 63.4 (C-1); m/z: Calculated for C46H₄₃F₃NaO₅ [M+Na]⁺: 755.3, found: 755.3.

Conversion of 6b to 6a: To a solution of compound 6b (5.33 g, 7.27 mmol) and pyridine (1.76 mL, 21.8 mmol) in dry DCM (20 mL), Tf₂O (2.45 L, 14.6 mmol) was added at −20° C. The reaction mixture was stirred at −20° C. for 30 minutes then at room temperature for 1 hour. The reaction was diluted with DCM (200 mL), washed with water (30 mL) and saturated aqueous NaHCO₃ (30 mL), dried over Na₂SO₄, filtered, and concentrated. The residue was dissolved in DMF (20 mL) and NaNO₂ (10 g, 145 mmol) was added followed by 18-crown-6 (576 mg, 2.18 mmol). The reaction mixture was stirred for 4 days at room temperature, then diluted with DCM (200 mL), washed with water (30 mL) and brine (30 mL), dried over Na₂SO₄, filtered, and concentrated in vacuo. The residue was purified by flash chromatography (petroleum ether/EtOAc, gradient 0-20%) to give compound 6a (3.05 g, 57%, 2 steps) as colorless wax.

Synthesis of compound 8: To a solution of compound 6a (11 g, 15 mmol) in DMF (50 mL), NaH (60% in mineral oil, 1.2 g, 30 mmol) was added at 0° C., followed by 2-(bromomethyl)naphthalene (6.65 g, 30.0 mmol). The reaction mixture was stirred for 18 hours at room temperature, then quenched by careful addition of water at 0° C. The formed suspension was taken up in Et₂O (500 mL), washed with saturated aqueous NH₄Cl (50 mL), dried over Na₂SO₄, filtered and concentrated in vacuo. The crude intermediate compound 7 was dissolved in 1,4-dioxane/water (2:1, 150 mL) and concentrated HCl (34 mL) was added dropwise. The reaction mixture was heated for 2 hours at 80° C., then cooled to room temperature and extracted with Et₂O (2×300 mL). The combined organic phases were washed with saturated aqueous NaHCO₃ (2×50 mL), dried over Na₂SO₄, filtered and concentrated in vacuo. The residue was purified by flash chromatography (petroleum ether/EtOAc, gradient 0-40%) to give compound 8 (7.57 g, 80%, 2 steps) as colorless wax. [α]_{D}^{>}−1.2 (c 0.8, CHCl₃); ¹H NMR (500 MHz, CDCl₃): δ=7.85-7.76 (m, 4H, Ar—H), 7.49-7.39 (m, 3H, Ar—H), 7.34-7.23 (m, 13H, Ar—H), 7.21-7.15 (m, 2H, Ar—H), 4.94 (A of AB, J=11.4 Hz, 1H, PhCH₂), 4.77 (B of AB, J=11.4 Hz, 1H, PhCH₂), 4.76 (A′ of A′B′, J=11.0 Hz, 1H, PhCH₂), 4.61 (A″ of A″B″, 11.7 Hz, 1H, PhCH₂), 4.59 (d, J=3.2 Hz, 2H, NapCH₂), 4.56 (B′ of A′B′, J=11.0 Hz, 1H, PhCH₂), 4.46 (B″ of A″B″, J=11.7 Hz, 1H, PhCH₂), 4.36 (m, 1H, H-5), 4.05 (dd, J=4.0, 5.4 Hz, 1H, H-4), 3.84 (m, 1H, H-3), 3.82-3.74 (m, 2H, H-1), 3.71 (m, 1H, H-2), 2.15 (t, J=6.1 Hz, OH); ¹³C NMR (126 MHz, CDCl₃): δ=138.0, 137.8, 137.7, 134.6, 133.2, 133.1, 128.5, 128.4, 128.3, 128.1, 128.1, 128.0, 127.9, 127,8, 127.8, 127.7, 127.7, 127.7, 126.8, 126.2, 126.1, 126.0, 125.9 (Ar—C, CF₃), 79.0 (C-2), 78.2 (C-3), 77.1 (C-4), 76.5-75.4 (m, C-5), 74.9, 74.4, 73.3, (3 PhCH₂), 72.4 (NapCH₂), 61.5 (C-1); ¹⁹F NMR (470 MHz, CDCl₃): δ=−72.2 (d, J=7.3 Hz, 3F, CF₃); ESI-MS: m/z: Calculated for C₃₈H₃₇F₃NaO₅[M+Na]⁺: 653.2, found: 653.3.

Synthesis of compound 9: To a solution of oxalyl chloride (0.2 ml, 2.37 mmol) in dry DCM (5 mL), dry DMSO (0.23 mL, 3.17 mmol) was added at −78° C. The reaction mixture was stirred for 30 minutes at −78° C., then a solution of compound 8 (1.00 g, 1.58 mmol) in DCM (5 mL) was added dropwise. The reaction was allowed to slowly warm up to −50° C. then the mixture was cooled to −78° C. and DIPEA (0.83 mL, 4.74 mmol) was added dropwise. The resulting mixture was allowed to warm up to −50° C. and was quenched with water (3 mL). The reaction was diluted with DCM (200 mL), the organic phase was washed with 0.5 M HCl (30 mL) and saturated aqueous NaHCO₃ (50 mL), dried over Na₂SO₄, filtered, and concentrated in vacuo. The crude product was purified by flash chromatography (petroleum ether/acetone, gradient 0-15%) to yield compound 9 (800 mg, 81%) as colorless wax. [α]_{D}²² −0.9 (c 1.25, CHCl₃); ¹H NMR (500 MHz, CDCl₃): δ=9.70 (d, J=0.8 Hz, 1H, H-1), 7.82-7.73 (m, 3H, Ar—H), 7.68 (s, 1H, Ar—H), 7.48-7.44 (m, 2H, Ar—H), 7.41 (dd, J=1.6, 8.4 Hz, 214, Ar—H), 7.33-7.21 (m, 13H, Ar—H), 7.11-7.07 (m, 21-1, Ar—H), 4.92 (A of AB, J=11.5 Hz, 1H, ArCH₂), 4.73 (A′ of A′B′, J=7.0 Hz, 1H, ArCH₂), 4.71 (B of AB, J=7.3 Hz, 1H, ArCH₂), 4.68 (A″ of A″B″, J=11.8 Hz, 1H, ArCH₂), 4.49 (B″ of A″B″, J=12.1 Hz, 1H, ArCH₂), 4.48 (B′ of A′B′, J=11.6 Hz, 1H, ArCH₂), 4.39 (A′″ of A′″B′″, J=11.5 Hz, 1H, ArCH₂), 4.31 (m, 1H, H-5), 4.21 (B′″ of A′″B′″, J=11.5 Hz, 1H, ArCH₂), 4.12-4.07 (m, 2H, H-2, H-3), 4.05 (dd, J=3.9, 5.9 Hz, 1H, H-4); ¹³C NMR (126 MHz, CDCl₃): δ=201.7 (C-1), 137.5, 137.1, 136.9, 134.3, 133.1, 133.1, 128.6, 128.4, 128.3, 128.2, 128.2, 128.1, 128.0, 127.9, 127.9, 127.7, 127.7, 127.5, 127.0, 126.1, 126.1, 125.9 (Ar—C, CF₃), 82.8 (C-2), 78.4 (C-3), 76.6 (C-4), 75.8 (q, J=28.7 Hz, C-5), 75.0, 74.3, 73.2, 73.0 (4 ArCH₂); ¹⁹F NMR (470 MHz, CDCl₃): δ=−71.8 (d, J=7.2 Hz, 3F, CF₃); ESI-MS: m/z: Calculated for C₃₈H₃₅F3NaO₅[M+Na]⁺: 651.7, found: 651.1.

Synthesis of compound 10: To a solution of compound 9 (800 mg, 1.27 mmol) in DCM (12 mL) water (0.1 ml) was added followed by the addition of DDQ (866 mg, 3.81 mmol) at 0° C. The reaction mixture was stirred for 6 hours at room temperature, then diluted with DCM (10 mL) and filtered through celite. The filtrate was washed with 10% aqueous Na₂CO₃ (10 mL) and brine (10 mL), dried over Na₂SO₄, filtered, and concentrated in vacuo. The crude product was purified by flash chromatography (petroleum ether/acetone, gradient 0-30%) to give compound 10 (320 mg, 51%) as a 3:1 mixture of α/β-anomers. ¹H NMR (500 MHz, CDCl₃): 1.0α: δ=7.39-7.26 (m, 15H, Ar—H), 5.34 (app t, J=3.0 Hz, 1H, H-1), 4.92-4.65 (m, 6H, 3 PhCH₂), 4.29 (q, J=6.6 Hz, 1H, H-5), 4.14 (d, J=2.1 Hz, 1H, H-4), 4.09 (dd, J=3.6, 9.9 Hz, 1H, H-2), 3.88 (dd, J=2.8, 9.9 Hz, 1H, H-3), 3.07 (d, J=2.5 Hz, 1H, OH); 10β: δ=7.39-7.26 (m, 15H, Ar—H), 4.92-4.65 (m, 7H, H-1, 3 PhCH₂), 4.07 (d, J=2.2 Hz, 1H, H-4), 3.83 (dd, J=7.3, 9.5 Hz, 1H, H-2), 3.76 (q, =6.3 Hz, 1H, H-5), 3.52 (dd, J=2.8, 9.5 Hz, 1H, H-3), 3.42 (d, J=7.7 Hz, 1H, OH); ¹³C NMR (126 MHz, CDCl₃): 10α: δ=138.1, 137.8, 137.7, 128.5,128.3, 128.2, 128.1, 128.0, 127.8, 127.7, 127.5 (Ar—C), 123.4 (q, J=280 Hz, C-6), 92.1 (C-1), 77.8 (C-3), 75.8 (C-2), 74.9, 73.9 (2 PhCH₂), 73,2 (C-4), 731 (PhCH₂), 69.5-68.7 (m, C-5); 10β: δ=138.2, 137.9, 137.5, 128.4, 128.3, 128.2, 128,0, 127.9, 127.8, 127.6 (Ar—C), 122.9 (q, J=280 Hz, C-6), 98.1 (C-1), 81.0 (C-3), 80.0 (C-2), 75.1, 74.8, 73.2 (3 PhCH₂), 73.2-72.4 (m, C-5), 72.2 (C-4); ¹⁹F NMR (470 MHz, CDCl₃): 10α: δ=−72.9 (d, J=−6.7 Hz, 3F, CF₃); 10β: δ=−72.8 (d, J=6.3 Hz, 3F, CF₃); EST-MS: m/z Calculated for C₂₇H₂₇F₃NaO₅ [M+Na]⁺: 511.2, found: 511.2.

Synthesis compound 11: To a solution of compound 10 (290 mg, 0.593 mmol) in dry DCM (3 mL) Cl₃CCN (0.59 mL, 5.94 mmol) was added followed by the addition of K₂CO₃ (409 mg, 2.96 mmol) at 0° C. The reaction mixture was stirred for 18 hours at 4° C., then filtered through a short pad of silica, which was washed with DCM (20 mL). The filtrate was concentrated in vacuo to obtain compound 11 (339 mg, 90%) as a 1:3.8 mixture of α/β-anomers. ¹H NMR (500 MHz, CDCl₃): 11α: δ−8.66 (s, 1H, NH), 7.39-7.24 (m, 15H, Ar—H), 6.64 (d, J=3.5 Hz, 1H, H-1), 4.95-4.65 (m, 6H, 3 PhCH₂), 4.31 (dd, J=3.5, 10.0 Hz, 1H, H-2), 4.27 (q, J=6.3 Hz, 1H, H-5), 4.19 (d, J=2.5 Hz, 1H, H-4), 3.98 (dd, J=2.7, 10.0 Hz, 1H, H-3); 11β: δ=8.73 (s, 1H, NH), 7.39-7.24 (m, 15H, Ar—H), 5.78 (d, J=8.1 Hz, 1H, H-1), 4.95-4.65 (m, 6H, 3 PhCH₂), 4.17 (dd, J=8.2, 9.6 Hz, 1H, H-2), 4.10 (d, J=2.5 Hz, 1H, H-4), 3.87 (q, J=6.0 Hz, 1H, H-5), 3.62 (dd, J=2.9, 9.7 Hz, 1H, H-3); ¹³C NMR (126 MHz, CDCl₃): 11α: δ=160.7 (C═N), 138.0, 137.7, 137.6, (Ar—C), 128.5-127.4 (Ar—C, C-6), 94.3 (C-1), 91.0 (CCl₃), 77.0 (C-3), 75.1 (C-2), 75.0, 73.4, 73.3 (3 PhCH₂), 73.2 (C-4), 71.7-70.8 (m, C-5); 11β: δ=161.2 (C═N), 137.9, 137.7, 137.6, (Ar—C), 128.5-127.4 (Ar—C, C-6), 98.1 (C-1), 90.6 (CCl₃), 81.1 (C-3), 77.3 (C-2), 75.4, 74.9 (2 PhCH₂), 73.8-72.9 (m, C5), 73.4 (PhCH₂), 72.3 (C-4); ¹⁹F NMR (470 MHz, CDCl₃): 11α: δ=−72.6 (d, J=6.2 Hz, 3F, CF₃); 11β: δ=−72.9 (d, J=6.4, 3F, CF₃); ESI-MS: m/z. Calculated for C₂₇H₂₇F₃NaO₅ [MH+Na]⁺: 511.2, found: 511.2.

Synthesis of compound 13: To a solution A compound 12 (1.00 g, 11.1 mmol) and Et₃N (1.87 mL, 13.3 mmol) in dry DCM (35 mL) BzCl (1.29 mL, 11.1 mmol) was added dropwise at −10° C. The mixture was allowed to slowly warm up to room temperature and was stirred for 18 hours. The reaction was diluted with. DCM (150 mL), washed with 1M HCl (20 mL) and saturated aqueous NaHCO₃ dried over Na₂SO₄, and concentrated. The crude product was purified by flash chromatography (petroleum ether/EtOAc, gradient 0-40%) to yield compound 13 (1.28 g, 59%) as colorless soil. [α]_{D}²² −43.5 (c 1.1, CHCl₃); ¹H NMR (500 MHz, CDCl₃): δ=8.05 (d, J=7.7 Hz, 2H, Ar—H), 7.57 (t, J=7.4 Hz, 1H, Ar—H), 7.45 (t, J=7.7 Hz, 2H, Ar—H), 5.03 (p, J=6.4 Hz, 1H, BzOCH), 3.91 (p, J=6.4 Hz, 1H, HOCH), 2.01 (br s, 1H, OH), 1.36 (d, J=6.4 Hz, 3H, BzOCHCH₃), 1.27 (d, J=6.4 Hz, 3H, HOCHCH₃); ¹³C NMR (126 MHz, CDCl₃): δ=166.4 (CO), 133.2, 129.7, 128.6 (Ar—C), 75.6 (BzOC), 70.6 (HOC), 19.2 (HOCHCH₃), 16.4 (BzOCHCH₃).

Synthesis of compound 15: To a solution of compound 14 (D. Schwizer et al., Chem Eur. J., 18, 1342-1351 (2012)) (500 mg, 2.04 mmol) in pyridine (7 mL) BzCl (0.35 mL, 3.07 mmol) was added dropwise at 0° C. The reaction was allowed to slowly warm up to room temperature and stirred for 18 hours. The mixture was diluted with petroleum ether (100 mL), washed with 0.5 M HCl (20 mL) and saturated aqueous NaHCO₃, dried over Na₂SO₄, and concentrated. The crude product was dissolved in MeOH (12 mL) and H₂SO₄ (81 μL) was added at 0° C. The reaction was stirred at room temperature for 18 h, neutralized with Et₃N (0.3 mL) and concentrated in vacuo. The crude product was purified by flash chromatography (petroleum ether/EtOAc, gradient 0-40%) to give compound 15 (364 mg, 76%, 2 steps) as colorless oil. [α]_{D}²² +38.6 (c 0.55, CHCl3); ¹H NMR (500 MHz, CDCl₃): δ=8.09 (app d, J=8.4 Hz, 2H, Ar—H), 7.57 (t, J=7.4 Hz, 1H, Ar—H), 7.46 (t, J=7.7 Hz, 2H, Ar—H), 4.70 (t, J=9.7 Hz, 1H, H-2), 3.67 (ddd, J=4.7, 9.2, 11.1 Hz, 1H, H-1), 2.12 (m, 1H, H-6a), 1.80-1.71 (m, 3H, H-4a, H-3, H-5a), 1.48-1.31 (m, 2H, H-5b, H-6b), 1.17 (m, 1H, H-4b), 0.97 (d, J=6.3 Hz, 3H, CH₃); ¹³C NMR (126 MHz, CDCl₃): δ=167.7 (CO), 133.1, 129.8, 128.4 (Ar—C), 83.6 (C-2), 73.7 (C-1), 36.5 (C-3), 33.9 (C-6), 33.1 (C-4), 23.2 (C-5), 18.2 (CH₃); ESI-MS: m/z: Calculated for C₁₄H₁₈NaO₃[M+Na]⁺: 257.1 found: 256.9.

Synthesis of compound 1: To a solution of compound 15 (360 mg, 1.53 mmol) and O-allyl-2,2,2-trichloroacetimidate (1.17 mL, 7.68 mmol) in dry DCM (3 mL) TfOH (0.135 mL, 1.53 mmol) was added dropwise at 0° C. The reaction was stirred at 0° C. for 0.5 h then at room temperature for 6 h. The reaction was quenched with Et₃N (0.3 mL) concentrated in vacuo and the crude residue was purified by flash chromatography (petroleum ether/EtOAc, gradient 0-20%) to give compound 16 (364 mg, 81%) as colorless oil. [α]_{D}²² +0.8 (c 0.94, CHCl₃); ¹H NMR (500 MHz, CDCl₃): δ=8.08 (app d, J=8.4 Hz, 2H, Ar—H), 7.55 (t, J=7.4 Hz, 1H, Ar—H), 7.45 (t, J=7.7 Hz, 2H, Ar—H), 5.72 (m, 1H, CH₂CHCH₂), 5.11 (dd, J =1.7, 17.2 Hz, 1H, CH₂CHCH₂O), 4.99 (dd, J=1.4, 10.4 Hz, 1H, CH₂CHCH₂O), 4.88 (m, 1H, H-2), 4.06 (dd, J=5.4, 13.0 Hz, 1H, CH₂CHCH₂O), 3.91 (dd, J=5.6, 13.0 Hz, 1H, CH₂CHCH₂O), 3.39 (ddd, J=4.6, 9.2, 11.0 Hz, 1H, H-1), 2.12 (m, 1H, H-6a), 1.78-1.65 (m, 3H, H-4a, H-3, H-5a), 1.44-1.29 (m, 2H, H-5b, H-6b), 1.16 (m, 1H, H-4b), 0.95 (d, J=6.4 Hz, 3H, CH₃); ¹³C NMR (126 MHz, CDCl₃): δ=166.3 (CO), 135.3 (CH₂CHCH₂O), 132.6, 129.6, 128.2 (Ar—C), 116.2 (CH₂CHCH₂O), 80.9 (C-2), 80.0 (C-1), 70.5 (CH₂CHCH₂O), 36.9 (C-3), 33.0 (C-4), 30.8 (C-6), 23.2 (C-5), 18.1 (CH₃); ESI-MS: m/z: Calculated for C₁₇H₂₂NaO₃ [M+Na]⁺: 297.4 found: 297.8.

Synthesis of compound 17: To a solution of compound 16 (330 mg, 1.2 mmol) in dry MeOH/DCM (1:2; 2 mL) NaOMe (1 M, 1.8 mL, 1.8 mmol) was added. The reaction was stirred for 3 d at room temperature, then diluted with MeOH (5 mL) and neutralized with Amberlite IR120 resin (H⁺ form). The resin was filtered off and the filtrate was concentrated in vacuo (40° C., 100 mbar). The crude residue was purified by flash chromatography (petroleum ether/Et₂O, gradient 0-30%) to give compound 17 (168 mg, 82%) as colorless oil. [α]_{D}²² −28.2 (c 0.84, CHCl₃); ¹H NMR (500 MHz, CDCl₃): δ=6.00-5.81 (m, 1H, CH₂CHCH₂), 5.28 (dd, J=1.6, 17.3 Hz, 1H, CH₂CHCH₂O), 5.18 (dd, J=1.3, 10.4 Hz, 1H, CH₂CHCH₂O), 4.16 (dd, J=5.5, 12.7 Hz, 1H, CH₂CHCH₂O), 3.96 (dd, J=5.8, 12.7 Hz, 1H, CH₂CHCH₂O), 3.12 (ddd, J=4.3, 8.8, 10.8 Hz, 1H, H-1), 3.01 (m, 1H, H-2), 2.71 (br s, 1H, OH), 2.07 (m, 1H, H-6a), 1.73-1.60 (m, 2H, H-4a, H-5a), 1.45 (m, 1H, H-3), 1.32-1.11 (m, 2H, H-5b, H-6b), 1.05 (d, J=6.4 Hz, 3H, CH₃), 1.00 (m, 1H, H-4b); ¹³C NMR (126 MHz, CDCl₃): δ=135.1 (CH₂CHCH₂O), 116.9 (CH₂CHCH₂O), 82.9 (C-1), 72.9 (C-2), 69.8 (CH₂CHCH₂O), 37.4 (C-3), 33.1 (C-4), 29.3 (C-6), 23.3 (C-5), 18.4 (CH₃); ESI-MS: m/z: Calculated for C₁₀H₁₈NaO₂ [M+Na]⁺: 193.1 found: 192.8.

Synthesis of compound 20: A suspension of compound 18 (A. Sherman et al., Carbohydr. Res., 338, 697-703 (2003)) (1.06 g, 3.35 mmol) and n-Bu₂SnO (1.67 g, 6.71 mmol) in dry MeOH (20 mL) was heated to reflux at 70° C. for 4 hours. The reaction was allowed to cool to room temperature, and dry toluene (100 mL) was added. The solvents were removed in vacuo and the residue was dried on high vacuum for 18 hours, then a solution of compound 19 (M. Qabar et al., Tetrahedron Lett, 39, 5895-5898 (1998)) (1.98 g, 5.03 mmol) in dry DME (20 mL) was added. To the resulting suspension anhydrous CsF (1.02 g, 6.71 mmol, dried on high vacuum at 100° C. for 18 hours) was added and the turbid mixture was stirred at room temperature for 18 hours. The reaction was quenched by addition of 1M K₂HPO₄ containing 10% KF (20 mL) at 0° C. The mixture was stirred at room temperature for 30 minutes, then extracted with DCM (3×100 mL). The combined organic phases were washed with saturated aqueous NaHCO₃ (50 mL), dried over Na₂SO₄, filtered and concentrated in vacuo. The crude product was purified by flash chromatography (petroleum ether/EtOAc, gradient 0-50%) to yield the desired intermediate (786 mg, 42% after two steps). To the solution of the intermediate (400 mg, 0.72 mmol) and DMAP (17.5 mg, 0.14 mmol) in pyridine (5 mL) was added BzCl (0.33 mL, 2.86 mmol) dropwise at 0° C. The mixture was stirred at room temperature for 18 hours then concentrated in vacuo. The crude residue was purified by flash chromatography (petroleum ether/EtOAc, gradient 0-30%) to yield compound 20 (467 mg, 85%). ¹H NMR (500 MHz, CDCl₃): δ=8.12-8.03 (m, 4H, Ar—H), 7.60-7.55 (m, 2H, Ar—H), 7.49-7.41 (m, 4H, Ar—H), 7.34-7.21 (m, 10H, Ar—H), 5.91 (d, J=3.2 Hz, 1H, H-4), 5.63 (app t, J=9.7 Hz, 1H, H-2), 5.18 (A of AB, J=12.1 Hz, 1H, PhCH₂), 5.18 (B of AB, J=12.1 Hz, 1H, PhCH₂), 4.55 (d, J=9.7 Hz, 1H, H-1), 4.53 (A′ of A′B′, J=11.8 Hz, 1H, PhCH₂), 4.47 (B′ of A′ B′, J=12.1 Hz, 1H, PhCH₂), 4.16 (dd, J=4.9, 7.8 Hz, 1H, Lac-H2), 4.87-3.80 (m, 2H, H-3, H-5), 3.65-3.53 (m, 2H, H-6), 2,84-2.70 (m, 2H, SCH₂CH₃), 1.41-1.14 (m, 7H, Cy-H, Lac-H3), 1.26 (t, J=7.5 Hz, 3H, SCH₂CH₃), 1.04 (m, 1H, Cy-H), 0.85-0.77 (m, 2H, Cy-H), 0.69 (m, 1H, Cy-H), 0.52-0.41 (m, 2H, Cy-H); ESI-MS: m/z: Calculated for C₄₅H₅₀NaO₉S [M+Na]⁺: 789.3, found: 789.3.

Synthesis of compound 23: A suspension of donor-compound 11 (140 mg, 0.221 mmol), acceptor-compound 21 (R. L. Wiseman et al., J. Am. Chem. Soc. 127, 5540-5551 (2005)) (41 mg, 0.663 mmol) and activated 4 Å molecular sieves in dry DCM (5 mL) was stirred at room temperature for 1 hour then TBDMS-OTf (15 p.L) was added at 0° C. The reaction was allowed to warm up to room temperature and stirred for 18 hours, then quenched with Et₃N (0.1 mL). The mixture was filtered through celite, and the filtrate was concentrated in vacuo. The crude residue was dissolved in dry MeOH/DCM (1:1, 4 mL) and 1M NaOMe (0.22 mL, 0.22 mmol) was added. The reaction was stirred for 3 hours at room temperature, diluted with MeOH (5 mL) and neutralized with Amberlite IR120 resin (H⁺ form). The resins were filtered off and the filtrate was concentrated in vacuo. The crude residue was purified by flash chromatography (petroleum ether/acetone, gradient 0-30%) to yield compound 23 (23 mg, 20%, 2 steps). [α]_{D}²² −52.8 (c 0.44, CHCl₃); ¹H NMR (500 MHz, CDCl₃): δ=7.41-7.25 (m, 15H, Ar—H), 4.95 (d, J=3.7 Hz, 1H, H-1), 4.91 (A of AB, J=10.9 Hz, 1H, PhCH₂), 4.84 (A′ of A′B′, J=11.5 Hz, 1H, PhCH₂), 4.82 (A″ of A″B″, J=11.1 Hz, 1H, PhCH₂), 4.73 (B″ of A″B″, J=11.7 Hz, 1H, PhCH₂), 4.67 (B of AB, J=10.9 Hz, 1H, PhCH₂), 4.66 (B′ of A′B′, J=11.8 Hz, 1H, PhCH₂), 4.18 (m, 1H, H-5), 4.16 (d, J=1.7 Hz, 1H, H-4), 4.12 (dd, J=3.7, 10.1 Hz, 1H, H-2), 3.93 (dd, J=2.8, 10.1 Hz, 1H, H-3), 3.82 (m, 1H, OCH₂CH₂O), 3.73 (t, J=4.4 Hz, 2H, OCH₂CH₂O), 3.62 (m, 1H, OCH₂CH₂O); ¹³C NMR (126 MHz, CDCl₃): δ=138.2, 137.9, 137.7, 128.5, 128.5, 128.3, 128.2, 128.2, 128.1, 127.8, 127.7, 127.5 (Ar—C), 98.7 (C-1), 78.2 (C-3), 75.7 (C-2), 74.9, 74.1 (2 PhdH₂), 73.4 (C-4), 73.2 (PhCH₂), 70.9 (OCH₂CH₂O), 69.0 (q, J=31.8 Hz, C-5); ¹⁹F NMR (470 MHz, CDCl₃): δ=−72.9 (d, J=6.7 Hz, 3F, CF₃); ESI-MS: m/z: Calculated for C₂₉H₃₁F₃NaO₆ [M+Na]⁺: 555.2, found: 555.1.

Synthesis of compound 24: A suspension of donor-compound 11 (140 mg, 0.221), acceptor-compound 22 (E. Santaniello et al., Tetrahedron: Asymm. 16, 1705-1708 (2005)) (79.2 mg, 0.442 mmol), and activated 4 Å molecular sieves in dry DCM (5 mL) was stirred at room temperature for 1 hour then TBDMS-OTf (15 μL) was added at 0° C. The reaction was allowed to warm up to room temperature and stirred for 18 hours, then quenched with Et₃N (0.1 mL). The mixture was filtered through celite, and the filtrate was concentrated in vacuo. The crude residue was dissolved in dry MeOH/DCM (1:1, 4 mL) and 1M NaOMe (0.22 mL, 0.22 mmol) was added. The reaction was stirred for 3 hours at room temperature, diluted with MeOH (5 mL) and neutralized with Amberlite IR120 resin (H⁺ form). The solids were filtered off and the filtrate was concentrated in vacuo. The residue was purified by flash chromatography (petroleum ether/acetone, gradient 0-30%) to give compound 24 (25 mg, 21%, 2 steps). [α]_{D}²² −62.7 (c 0.44, CHCl₃); ¹H NMR (500 MHz, CDCl₃): δ=7.39-7.26 (m, 15H, Ar—H), 4.93 (d, J=3.7 Hz, 1H, H-1), 4.90 (A of AB, J=10.9 Hz, 1H, PhCH₂), 4.84 (A′ of A′B′, J=11.8 Hz, 1H, PhCH₂), 4.81 (A″ of A″B″, J=11.7 Hz, 1H, PhCH₂), 4.73 (B″ of A″B″, J=11.7 Hz, 1H, PhCH₂), 4.67 (B of AB, J=10.9 Hz, 1H, PhCH₂), 4.66 (B′ of A′B′, J=11.8 Hz, 1H, PhCH₂), 4.19-4.14 (m, 2H, H-4, H-5), 4.11 (dd, J=3.7, 10.1 Hz, 1H, H-2), 3.97 (m, 1H, OCH₂CH(CH₃)O), 3.93 (dd, J=2.8, 10.1 Hz, 1H, H-3), 3.93 (dd, J=3.1, 10.9 Hz, 1H, OCH₂CH(CH₃)O), 3.47 (dd, J=7.8, 10.9 Hz, 1H, OCH₂CH(CH₃)O), 2.70 (br s, 1H, OH), 1.14 (d, J=6.4 Hz, 3H, OCH₂CH(CH₃)O); ¹³C NMR (126 MHz, CDCl₃): δ=138.1, 137.9, 137.7, 128.5, 128.5, 128.3, 128.2, 128.1, 128.0, 127.8, 127.7, 127.5 (Ar—C, C-6), 97.9 (C-1), 78.1 (C-3), 75.2 (C-2), 74.9 (PhCH₂), 74.2 (OCH₂CH(CH₃)O), 74.1 (PhCH₂), 73.4 (C-4), 73.2 (PhCH₂), 69.1 (q, J=31.8 Hz, C-5), 65.5 (OCH₂CH(CH₃)O), 18.7 (OCH₂CH(CH₃)O); ¹⁹F NMR (470 MHz, CDCl₃): δ=−72.9 (d, J=6,8 Hz, 3F, CF₃); ESI-MS: m/z: Calculated for C₃₀H₃₃F₃NaO₆ [M+Na]⁺: 569.2 found: 569.2.

Synthesis compound 25: A suspension of donor-compound 20 (40 mg, 0.052 mmol), acceptor-compound 23 (23 mg, 0.043 mmol) and activated 4 Å molecular sieves (0.5 g) in dry DCM (3 mL) was stirred at room temperature for 3 hours. In a second flask a suspension of dimethyl(methylthio)sulfonium tritlate (DMTST, 55 mg, 0.215 mmol) and activated 4 Å molecular sieves (0.2 g) in dry DCM (2 mL) was stirred at room temperature for 3 hours and then added via syringe to the first flask. The reaction mixture was stirred at room temperature for 18 hours, filtered through celite and the celite was washed with DCM (50 mL). The filtrate was washed with saturated aqueous NaHCO₃ (20 mL), dried over Na₂SO₄, filtered and concentrated. The crude product was purified by flash chromatography (petroleum ether/EtOAc, gradient 0-30%) to obtain compound 25 (40 mg, 75%). [α]_{D}²² −9.4 (c 0.5, CHCl₃); ¹H NMR (500 MHz, CDCl₃): δ=8.10 (d, J=7.2 Hz, 2H, Ar—H), 8.02 (d, J=7.3 Hz, 2H, Ar—H), 7.59-7.48 (m, 2H, Ar—H), 7.47-7.40 (m, 2H, Ar—H), 7.40-7.19 (m, 27H, Ar—H), 5.82 (d, J=3.3 Hz, 1H, Gal-H4), 5.59 (dd, J=8.1, 9.8 Hz, 1H, Gal-H2), 5.15 (A of AB, J=12.1 Hz, 1H, PhCH₂), 5.07 (B of AB, J=12.1 Hz, 1H, PhCH₂), 4.85 (d, J=3.6 Hz, 1H, Fuc-H1), 4.81 (A′ of A′B′, J=10.9 Hz, 1H, PhCH₂), 4.70 (A″, A′″ of A″B″, A′″B′″, J=11.8 Hz, 2H, PhCH₂), 4.63 (d, J=8.0 Hz, 1H, Gal-H1), 4.56 (B′, B″ of A′B′, A″B″, J=11.1 Hz, 2H, PhCH₂), 4.52 (A″″ of A″″B″″, J=11.8 Hz, 1H, PhCH₂), 4.51 (B′″ of A′″B′″, J=11.6 Hz, 1H, PhCH₂), 4.46 (B″″ of A″″ B″″, J=11.8 Hz, 1H, PhCH₂), 4.12 (dd, 5.0, 7.6 Hz, 1H, Lac-H2), 4.03-3.88 (m, 3H, Fuc-H2, Fuc-H4, Fuc-H5), 3.83-3.75 (m, 4H, Gal-H3, Gal-H5, OCH₂CH₂O), 3.73-3.65 (m, 3H, Fuc-H3, OCH₂CH₂O), 3.63-3.55 (m, 2H, Gal-H6), 1.40-1,13 (m, 6H, Cy-H, Lac-H3), 1.03 (m, 1H, Cy-H), 0.94-0.73 (m, 3H, Cy-H), 0.66 (m, 1H, Cy-H), 0.50-0.40 (m, 2H, Cy-H); ¹³C NMR (126 MHz, CDCl₃): δ=172.4 (Lac-C1), 165.9, 164.7 (Bz-CO), 138.5, 138.3, 137.9, 137.9, 135.6, 133.3, 133,0, 130.1, 129.8, 129.7, 128.6, 128.5, 128.4, 128.4, 128.2, 128.0, 127.8, 127.8, 127.7, 127.7, 127.6, 127.3 (Ar—C, Fuc-C6), 100.8 (Gal-C1), 98.5 (Fuc-C1), 78.4 (Lac-C2), 78.1 (Fuc-C3), 77.6 (Gal-C3), 75.8 (Fuc-C2), 74.8, 73.7 (2 PhCH₂), 73.6 (Gal-C5), 73.4, 73.4, 73.4 (Fuc-C4, 2 PhCH₂), 72.6 (Gal-C2), 70.2 (Gal-C4), 69.2 (Gal-C6), 69.1-68.3 (m, Fuc-C5), 68.3, 67.6 (OCH₂CH₂O), 66.6 (PhCH₂), 40.4, 33.3, 32.8, 26.1, 25.8, 25.5 (Cy-C, Lac-C3); ¹⁹F NMR (470 MHz, CDCl₃): δ=−72.3 (d, J=6.7 Hz, 3F, CF₃); ESI-MS: m/z Calculated for C₇₂H₇₅F₃NaO₁₅ [M+Na]⁺: 1260.4, found: 1260.3.

Synthesis of compound 20: A suspension of donor-compound 20 (40 mg, 0.052 mmol), acceptor-compound 24 (20 mg, 0.036 mmol) and activated 4 Å molecular sieves (0.5 g) in dry DCM (3 mL) was stirred at room temperature for 3 hours. In a second flask a suspension of dimethyl(methylthio)sulfonium triflate (DMTST, 47 mg, 0.182 mmol) and activated 4 Å molecular sieves (0.2 g) in dry DCM (2 mL) was stirred at room temperature for 3 hours and then added via syringe to the first flask. The reaction was stirred at room temperature for 18 hours, filtered through celite and the celite was washed with DCM (50 mL). The filtrate was washed with saturated aqueous NaHCO₃ (20 mL), dried over Na₂SO₄, filtered and concentrated. The crude product was purified by flash chromatography (petroleum ether/EtOAc, gradient 0-40%) to yield compound 26 (40 mg, 87%). [α]_{D}²² −28.1 (c 0.70, CHCl₃); ³H NMR (500 MHz, CDCl₃): δ=8.10 (d, J=7.2 Hz, 2H, Ar—H), 8.03 (d, J=7.3 Hz, 2H, Ar—H), 7.54 (dt, J=7.4, 14.5 Hz, 2H, Ar—H), 7.42 (td, J=2.9, 7.7 Hz, 4H, Ar—H), 7.34-7.14 (m, 25H, Ar—H), 5.82 (d, 3.3 Hz, 1H, Gal-H4), 5.53 (dd, J=8.2, 9.7 Hz, 1H, Gal-H2), 5.14 (A of AB, J=12.1 Hz, 1H, PhCH₂), 5.06 (B of AB, J=12.1 Hz, 1H, PhCH₂), 4.94 (d, J=3.5 Hz, 1H, Fuc-H1), 4.81 (A′ of A′ B′, J=10.9 Hz, 1H, PhCH₂), 4.71 (A″ of A″B″, J=11.7 Hz, 1H, PhCH₂), 4.70 (A′″ of A′″B′″, J=11.9 Hz, 1H, Ph-CH₂), 4.63 (d, J=9.2 Hz, 1H, Gal-H1), 4.61 (B′″ of A′″B′″, J=11.9 Hz, 1H, PhCH₂), 4.57 (B′ of A′B′, J=10.9 Hz, 1H, PhCH₂), 4.56 (B″ of A″B″, J=11.7 Hz, 1H, PhCH₂), 4.48 (A″″ of A″″B″″, J=11.8 Hz, 1H, PhCH₂), 4.41 (B″″ of A″″B″″, J=11.8 Hz, 1H, PhCH₂), 4.24 (q, J=6.4 Hz, 1H, Fuc-H5), 4.11 (dd, J=5.0, 7.8 Hz, 1H, Lac-H2), 4.05-4.00 (m, 2H, Fuc-H2, Fuc-H4), 3.95 (m, 1H, OCH₂CH(CH₃)O), 3.88 (dd, J=2.8, 10.0 Hz, 1H, Fuc-H3), 3.80-3.75 (m, 2H, Gal-H3, Gal-H5), 3.72 (dd, J=6.3, 10.7 Hz, 1H, OCH₂CH(CH₃)O), 3.60-3.53 (m, 2H, Gal-H6), 3.43 (dd, J=4.6, 10.7 Hz, 1H, OCH₂CH(CH₃)O), 1.41-1.14 (m, 6H, Cy-H, Lac-H3), 1.03 (m, 1H, Cy-H), 1.00 (d, J=6.4, 3H, OCH₂CH(CH₃)O), 0.94-0.63 (m, 4H, Cy-H), 0.66 (m, 1H, Cy-H), 0.53-0.44 (m, 2H, Cy-H); ¹³C NMR (126 MHz, CDCl₃): δ=172.4 (Lac-C1), 165.9, 164.7 (Bz-CO): 138.5, 138.4, 138.0, 137.9, 135.6, 133.1, 133.0, 130.1, 130.1, 129.9, 129.8, 129.6, 128.5, 128.5, 128.4, 128.4, 128.3, 128.3, 128.3, 128.2, 128.1, 128.0, 127.7, 127.7, 127.6, 127.5, 127.2 (Ar—C, Fuc-C6), 100.9 (Gal-C1), 97.6 (Fuc-C1), 78.2, 78.1 (Lac-C2, Fuc-C3), 77.6 (Gal-C3), 75.8 (Fuc-C2), 75.3 (OCH₂CH(CH₃)O), 74.8, 73.7 (2 PhCH₂), 73.6 (Fuc-C4), 73.4 (Gal-C5), 73.3, 73.0 (2 PhCH₂), 73.0 (Gal-C2), 71.7 (OCH₂CH(CH₃)O), 70.1 (Gal-C4), 69.1 (Gal-C6), 69.1-68.3 (m, Fuc-C5), 66.6 (PhCH₂), 40.4, 33.4, 33.3, 32.7, 26.1, 25.7, 25.5 (Cy-C, Lac-C3), 17.3 (OCH₂CH(CH₃)O); ¹⁹F NMR (470 MHz, CDCl₃): δ=−72.6 (d, J=6.8 Hz, 3F, CF₃); ESI-MS: m/z: Calculated for C₇₃H₇₇F₃NaO₁₅ [M+Na]⁺: 1273.5, found: 1273.6.

Synthesis of compound 27: A suspension of compound 25 (38 mg, 0.031 mmol) and Pd(OH)₂/C (50 mg, 10% Pd) in THF (4 mL) was hydrogenated (1 bar H₂) for 2 hours at room temperature. The reaction mixture was filtered through a double layer of filter paper and concentrated in vacuo. The crude residue was dissolved in dry MeOH (0.3 mL) and 1M aqueous LiOH (0.3 mL, 0.30 mmol) was added. The reaction was heated via microwave irradiation to 50° C. for 10 h, then diluted with MeOH (5 mL) and neutralized with Amberlite IR120 resin (H⁺ form). The resin was filtered off and the filtrate was concentrated in vacuo. The crude residue was dissolved in water (1 mL) and filtered through a short column of Dowex 50 (Na⁺ form) ion exchange resin. The filtrate was concentrated and the crude product was purified by reversed phase column chromatography (C18, H₂O/MeOH, gradient 0-100%) to give compound 27, which was microfiltered and lyophilized from water (8.0 mg, 45%, 2 steps). ¹H NMR (500 MHz, D₂O): δ=5.15 (d, J=3.8 Hz, 1H, Fuc-H1), 4.57 (q, J=6.8 Hz, 1H, Fuc-H5), 4.47 (d, J=8.0 Hz, 1H, Gal-H1), 4.30 (d, J=3.1 Hz, 1H, Fuc-H4), 4.14 (m, 1H, OCH₂CH₂O), 4.03-3.90 (m, 5H, Lac-H2, Fuc-H3, Gal-H4, OCH₂CH₂O), 3.88 (dd, J=3.8, 10.3 Hz, 1H, Fuc-H2), 3.84-3.75 (m, 3H, Gal-H6, OCH₂CH₂O), 3.70 (dd, J=4.1, 7.8 Hz, 1H, Gal-H5), 3.63 (dd, J=8.2, 9.4 Hz, 1H, Gal-H2), 3.44 (dd, J=3.2, 9.6 Hz, 1H, Gal-H3), 1.81 (m, 1H, Cy-H), 1.75-1.52 (m, 7H, Lac-H3, Cy-H), 1.30-1.12 (m, 3H, Cy-H), 1.03-0.87 (m, 2H, Cy-H); ¹³C NMR (126 MHz, CDCl₃): δ=182.5 (Lac-C1), 102.7 (Gal-C1), 98.9 (Fuc-C1), 82.8 (Gal-C3), 79.3 (Lac-C2), 74.6 (Gal-C5), 69.9 (Gal-C2), 68.7 (OCH₂CH₂O), 68.5 (Fuc-F3), 68.7-68.2 (m, Fuc-C5), 67.7 (Fuc-C2), 67.4 (OCH₂CH₂O), 67.1 (Fuc-C4), 66.2 (Gal-C4), 61.1 (Gal-C6), 41.2, 33.6, 33.2, 31.9, 26.1, 26.0, 25.7 (Cy-C, Lac-C3); ¹⁹F NMR (470 MHz, CDCl₃): δ=−73.1 (d, J=7.0 Hz, 3F, CF₃); HR-MS: m/z: Calculated for C₂₃H₃₆F₃Na₂O₁₃ [M+Na]⁺: 623.1903, found: 623.1906.

### Example 2

### Synthesis of Compound 28

Synthesis of compound 28: A suspension of compound 26 (25 mg, 0.020 mmol) and Pd(OH)₂/C (50 mg, 10% Pd) in THE (5 mL) was hydrogenated (1 bar H₂) for 4 hours at room temperature. The reaction mixture was filtered through a double layer of filter paper and concentrated in vacuo. The crude residue was dissolved in dry MeOH (0.4 mL) and 1M aqueous LiOH (0.4 mL, 0.40 mmol) was added. The reaction was heated via microwave irradiation to 50° C. for 3 hours, then diluted with MOH (5 mL) and neutralized with Amberlite IR120 resin (H⁺ form). The solids were filtered off and the filtrate was concentrated in vacuo. The crude residue was dissolved in water (1 mL) and filtered through a short column of Dowex 50 (Na⁺ form) ion exchange resin. The filtrate was concentrated and the crude product was purified by reversed phase column chromatography (C18, H₂O/MeOH, gradient 0-100%) to yield compound 28, which was microfiltered and lyophilized from water (6.8 mg, 55%, 2 steps). ¹H NMR (500 MHz, D₂O): δ=5.13 (d, J=3.7 Hz, 1H, Fuc-H1), 4.58 (q, J=6.8 Hz, 1H, Fuc-H5), 4.52 (d, J=8.0 Hz, 1H, Gal-H1), 4.31 (d, J=3.0 Hz, 1H, Fuc-H4), 4.26-4.16 (m, 1H, OCH₂CH(CH₃)O), 4.05-3.99 (m, 1H, Lac-H2), 3.98-3.91 (m, 2H, Fuc-H3, Gal-H4), 3.88 (dd, J=3.8, 10.3 Hz, 11I, Fuc-H2), 3.82-3.73 (m, 3H, Gal-H6, OCH₂CH(CH₃)O), 3.72-3.65 (m, 2H, Gal-H5, OCH₂CH(CH₃)O), 3.59 (dd, J=8.2, 9.4 Hz, 1H, Gal-H2), 3.43 (dd, 3.1, 9.6 Hz, 1H, Gal-H3), 1.84-1.78 (m, 1H, Cy-H), 1.75-1.53 (m, 7H, Lac-H3, Cy-H), 1.27 (d, J=6.5 Hz, 3H, OCH₂CH(CH₃)O), 1.33-1.11 (m, 3H, Cy-H), 1.05-0.87 (m, 2H, Cy-H); ¹³C NMR (126 MHz, CDCl₃): δ=182.4 (Lac-C1), 101.1 (Gal-C1), 98.5 (Fuc-C1), 82.5 (Gal-C3), 79.3 (Lac-C2), 74.5 (Gal-C5), 74.2 (OCH₂CH(CH₃)O), 71.3 (OCH₂CH(CH₃)O), 70.0 (Gal-C2), 69.0-68.0 (m, Fuc-C5), 68.6 (Fuc-F3), 67.8 (Fuc-C2), 67.2 (Fuc-C4), 66.2 (Gal-C4), 61.1 (Gal-C6), 41.2, 33.6, 33.2, 31.9, 26.1, 25.9, 25.7 (Cy-C, Lac-C3), 15.7 (OCH₂CH(CH₃)O); ¹⁹F NMR (470 MHz, CDCl₃): δ=−73.15 (d, J=7.0, 3F, CF₃); HR-MS: m/z: Calculated for C₂₄H₃₈F₃Na₂O₁₃ [M+Na]⁺: 637.2060, found: 637.2061.

### Example 3

### Synthesis of Compound 33

Synthesis of compound 29: A suspension of donor-compound 11 (282 mg, 0.446), acceptor-compound 13 (104 mg, 0.535 mmol), and activated 4 Å molecular sieves (1 g) in DCM (7 mL) was stirred at room temperature for 1 h then. TBDMS-OTf (30 μL) was added at 0 CC. The reaction was allowed to warm to room temperature and stirred for 18 hours, then it was quenched with Et₃N (0.3 mL). The mixture was filtered through celite, and the filtrate was concentrated in maw. The crude residue was dissolved in dry MeOH/DCM (1:1, 6 mL) and 1M NaOMe (0.45 mL, 0.446 mmol) was added. The reaction was stirred for 18 h at room temperature, diluted with MeOH (5 mL) and neutralized with Amberlite IR120 resin (H⁺ form). The solids were filtered off and the filtrate was concentrated in vacuo. The crude residue was purified by flash chromatography (petroleum ether/acetone, gradient 0-40%) to give compound 29 (45 mg, 18%, 2 steps). [α]_{D}²² −66.8 (c 1.00, CHCl₃); ¹H NMR (500 MHz, CDCl₃): δ=7.38-7.24 (m, 15H, Ar—H), 5.09 (d, J=3.8 Hz, 1H, H-1), 4.90 (A of AB, J=10.9 Hz, 1H, PhCH₂), 4.82 (A′ of A′B′, J=11.8 Hz, 1H, PhCH₂), 4.81 (A″ of A″B″, J=11.6 Hz, 1H, PhCH₂), 4.72 (B″ of A″B″, J=11.6 Hz, 1H, PhCH₂), 4.67 (B′ of A′B′, J=11.8 Hz, 1H, PhCH₂), 4.66 (B of AB, J=10.9 Hz, 1H, PhCH₂), 4.31 (q, J=6.7 Hz, 1H, H-5), 4.16 (d, J=2.1 Hz, 1H, H-4), 4.13 (dd, J=3.8, 10.1 Hz, 1H, H-2), 3.93 (dd, J=2.8, 10.1 Hz, 1H, H-3), 3.68 (m, HOCH), 3.57 (m, 1H, CH₃CH-Fuc), 2.76 (d, J=3.8 Hz 1H, OH), 1.16 (d, J=6.3 Hz, 6H, 2 CH₃); ¹³C NMR (126 MHz, CDCl₃): δ=138.3, 138.0, 138.0, 128.6, 128.6, 128.4, 128.3, 128.3, 128.1, 127.9, 127.8, 127.6 (Ar—C, C-6), 94.2 (C-1), 78.6 (CH₃CH-Fuc), 78.5 (C-3), 75.5 (C-2), 75.0, 74.1 (2 PhCH₂), 73.6 (C-4), 73.4 (PhCH₂), 70.2 (HOCH), 69.2 (q, J=31.8 Hz, C-5), 19.4, 15.5 (2 CH₃); ¹⁹F NMR (470 MHz, CDCl₃): δ=−72.9 (d, J=6.8 Hz, 3F, CF₃); ESI-MS: m/z; Calculated for C₃₁H₃₅F₃NaO₆ [M+Na]⁺: 583.2 found: 583.2.

Synthesis of compound 31: A suspension of donor-compound 30 (S. Nilsson et al., Glycoconj. J, 8, 9-15 (1991)) (84.0 mg, 0.160 mmol), acceptor-compound 29 (20 mg, 0.080 mmol) and activated 4 Å molecular sieves (0.5 g) in dry DCM (3 mL) was stirred at room temperature for 3 hours. In a second flask a suspension of dimethyl(methylthio)sulfonium triflate (DMTST, 103 mg, 0.40 mmol) and activated 4 Å molecular sieves (0.2 g) in DCM (2 mL) was stirred at room temperature for 3 hours then added via syringe to the first flask. The reaction was stirred at room temperature for 18 hours, then neutralized with Et₃N (0.3 mL). The mixture was filtered through celite, the celite was washed with DCM (50 mL) and the filtrate was concentrated in vacua. The crude product was purified by flash chromatography (petroleum ether/EtOAc, gradient 0-40%). The glycosylated intermediate was dissolved in dry MeOH/DCM (1:1, 4 mL) and 1M NaOMe (0.15 mL, 0.15 mmol) was added. The mixture was stirred for 18 hours at room temperature, diluted with MeOH (5 mL) and neutralized with Amberlite IR120 resin (H⁺ form). The resin was filtered off and the filtrate was concentrated in vacua. The residue was purified by flash chromatography (petroleum ether/acetone, gradient 0-50%) to give compound 31 (53 mg, 82%, 2 steps). [α]_{D}²² −81.1 (c 1.00, CHCl₃); ¹H NMR (500 MHz, CDCl₃): δ=7.52 (d, J=7.0 Hz, 2H, Ar—H), 7.36-7.13 (m, 18H, Ar—H), 5.62 (s, 1H, PhCH), 5.02 (q, J=6.7 Hz, 1H, Fuc-H5), 4.86 (br s, 1H, Fuc-H1), 4.78 (A of AB, J=12.2 Hz, 1H, PhCH₂), 4.69 (A′ of A′B′, J=11.3 Hz, 1H, PhCH₂), 4.61 (B of AB, J=12.2 Hz, 1H, PhCH₂), 4.58 (B′ of A′B′, J=11.3 Hz, 1H, PhCH₂), 4.35 (A″ of A″B″, J=10.8 Hz, 1H, PhCH₂), 4.34 (dd, J=1.3, 12.4 Hz, 1H, Gal-H6), 4.29 (d, J=7.3 Hz, 1H, Gal-H1), 4.22 (d, J=2.7 Hz, 1H, Gal-H4), 4.08 (dd, J=1.7, 12.4 Hz, 1H, Gal-H6), 3.96-3.93 (m, 2H, Fuc-H2, Fuc-H4), 3.92 (B″ of A″B″, J=10.8 Hz, 1H, PhCH₂), 3.87-3.82 (m, 2H, Fuc-H3, CH₃CH), 3.78-3.67 (m, 3H, Gal-H3, Gal-H2, CH₃CH.), 3.44 (d, J=1.0 Hz, 1H, Gal-H5), 2.58 (d, J=8.0 Hz, 1H, OH), 2.42 (d, J=1.6 Hz, 1H, OH), 1.15 (d, J=6.4 Hz, 1H, CH₃), 1.11 (d, J=6.4 Hz, 1H, CH₃); ¹³C NMR (126 MHz, CDCl₃): δ=139.1, 138.7, 138.5, 137,5, 128.9, 128.3, 128.2, 128.1, 128.1, 128.0, 127.8, 127.7, 127.4, 127.3, 127.2, 125.9 (Ar—C, Fuc-C6), 101.2 (Gal-C1), 100.5 (PhCH), 94.5 (Fuc-C1), 78.4, 78.2 (CH₃CH, Fuc-C3), 75.5, 75.4 (Gal-C4, Fuc-C4), 74.8 (PhCH₂), 74.4 (CH₃CH), 74.3 (Fuc-C2), 73.7 (PhCH₂), 72.6 (Gal-C3), 72.4 (PhCH₂), 71.6 (Gal-C2), 69.3 (Gal-C6), 68.8-67.8 (m, Fuc-C5), 66.8 (Gal-C5), 16.8, 15.5 (2 CH₃); ¹⁹F NMR (470 MHz, CDCl₃): δ=−72.0 (d J=7.0 Hz, 3F, CF₃); ESI-MS: m/z: Calculated for C₄₄H₄₉F₃NaO₁₁ [M+Na]⁺: 833.3, found: 833.3.

Synthesis of compound 33: suspension of compound 31 (52 mg, 0.064 mmol) and n-Bu₂SnO (24 mg, 0.096 mmol) in dry MeOH (4 mL) was heated to reflux at 70° C. for 4 h. The reaction was allowed to cool to room temperature, and dry toluene (20 mL) was added. The solvents were removed in vacua and the residue was dried on high vacuum for 4 h, then a solution of compound 19 (M. Qabar et al., Tetrahedron Lett. 39, 5895-5898 (1998)) (76 mg, 0.192 mmol) in dry DME (3 mL) was added. To the resulting suspension anhydrous CsF (29 mg, 0.192 mmol, dried on high vacuum at 110° C. for 5 hours) was added and the turbid mixture was stirred at room temperature for 18 hours. The reaction was quenched by addition of 1M K₂HPO₄ containing 10% KF (2 mL) at 0° C. The mixture was stirred at room temperature for 30 minutes, then extracted with DCM (3×30 mL). The combined organic phases were washed with saturated aqueous NaHCO₃ (20 mL), dried over Na₂SO₄, filtered and concentrated in vacua. The crude product was purified by flash chromatography (toluene/EOAc, gradient 0-30%) to yield a mixture of benzylester 32a and lactone 32b in 2.5:1 ratio (41 mg, 63%, 2 steps). ESI-MS: m/z: Calculated for 32a C₆₀H₆₉F₃NaO₁₃ [M+Na]⁺: 1077.5, found: 1077.5; Calculated for 32b C₅₃H₆₁F₃NaO₁₂ [M+Na]⁺: 969.4, found: 969.4. To a mixture of 32a-b (20 mg, 0.020 mmol) in THE/H₂O (1:1, 1 mL) 1M aqueous NaOH (0.1 mL, 0.1 mmol) was added. The reaction was stirred at room temperature for 18 h, then neutralized with Amberlite IR120 resin (H⁺ form). The resin was filtered off and the filtrate was concentrated in vacua. The crude residue was dissolved in THF (4 mL) and Pd(OH)₂/C (10 mg, 10% Pd) was added. The suspension was hydrogenated (1 bar H₂) for 2 hours at room temperature. The reaction mixture was filtered through a double layer of filter paper and concentrated in vacua. The crude residue was dissolved in water (1 mL) and filtered through a short column of Dowex 50 (Na⁺ form) ion exchange resin. The filtrate was concentrated and the crude product was purified by reversed phase column chromatography (C18, H₂O/MeCN, gradient 0-40%) to give compound 33, which was microfiltered and lyophilized from water (4.1 mg, 33%, 2 steps). NMR (500 MHz, D₂O): δ=5.18 (d, J=3.8 Hz, 1H, Fuc-H1), 4.87 (q, J=6.9 Hz, 1H, Fuc-H5), 4.46 (d, J=8.0 Hz, 1H, Gal-H1), 4.30 (d, J=3.0 Hz, 1H, Fuc-H4), 4.01-3.92 (m, 4H, OCH(CH₃), Lac-H2, Fuc-H3, Gal-H4), 3.90-3.82 (m, 2H, OCH(CH₃), Fuc-H2), 3,73-3.73 (m, 2H, Gal-H6), 3.64 (t, J=6.5 Hz, 1H, Gal-H5), 3.57 (dd, J=8.0, 9.6 Hz, 1H, Gal-H2), 3.41 (dd, J=3.2, 9.6 Hz, 1H, Gal-H3), 1.81 (m, 1H, Cy-H), 1.75-1.53 (m, 7H, Lac-H3, Cy-H), 1.33-1.11 (m, 9H, OCII(CH₃), Cy-H), 1.04-0.87 (m, 2H, Cy-H); ¹³C NMR (126 MHz, CDCl₃): δ=182.8 (Lac-C1), 101.0 (Gal-C1), 95.9 (Fuc-C1), 83.0 (Gal-C3), 79.3 (Lac-C2), 77.7, 75.5 (OCH(CH₃)), 74.3 (Gal-C5), 69.9 (Gal-C2), 68.6 (Fuc-F3), 68.8-68.4 (m, Fuc-C5), 67.6 (Fuc-C2), 67.2 (Fuc-C4), 66.2 (Gal-C4), 61.4 (Gal-C6), 41.2, 33.6, 33.3, 31.9, 26.2, 26.0, 25.7 (Cy-C, Lac-C3), 15.3, 14.2 (OCH(CH₃)); ¹⁹F NMR (470 MHz, CDCl₃): δ=−72.9 (d, J=6.9 Hz, 3F, CF₃); ESI-MS: m/z: Calculated for C₂₅H₄₁F₃NaO₁₃ [M+Na]⁺: 629.2, found: 629.2.

### Example 4

### Synthesis of Compound 37

Synthesis of compound 35: A suspension of donor-compound 11 (200 mg, 0.316 mmol), acceptor-compound 34 (183 mg, 1.58 mmol) and activated 4 Å molecular sieves (0.5 g) in DCM (5 mL) was stirred at room temperature for 1 hour then TBDMS-OTf (21 μL) was added at 0° C. The reaction was allowed to warm to room temperature and stirred for 18 hours, then it was quenched with Et₃N (0.3 mL). The mixture was filtered through celite, and the filtrate was concentrated in vacua. The residue was purified by flash chromatography (petroleum ether/acetone, gradient 0-30%) to give compound 35 (52 mg, 28%). [α]_{D}²² −16.5 (c 0.50, CHCl₃); ¹H NMR (500 MHz, CDCl₃): δ=7.39-7.23 (m, 15H, Ar—H), 5.14 (d, J=3.7 Hz, 1H, H-1), 4.90 (A of AB, J=10.8 Hz, 1H, PhCH₂), 4.82 (A′ of A′B′, J=11.6 Hz, PhCH₂), 4.81 (A″ of A″B″, J=11.7 Hz, 1H, PhCH₂), 4.73 (B′ of A′ B′, J=11.6 Hz, PhCH₂), 4.66 (B″ of A″B″, J=11.7 Hz, 1H, PhCH₂), 4.66 (B of AB, J=10.8 Hz, 1H, PhCH₂), 4.66 (q, J=6.7 Hz, 1H, H-5), 4.18 (d, J=2.0 Hz, 1H, 11-4), 4.12 (dd, J=3.8, 10.1 Hz, 1H, H-2), 3.98 (dd, J=2.9, 10.1 Hz, 1H, H-3), 3.51 (m, 1H, Cy-H1), 3.38 (m, 1H, Cy-H2), 2.74 (d, J=2.9 Hz, 1H, OH), 2.05-4.96 (m, 2H, Cy-H), 1.77-1.68 (m, 2H, Cy-H), 1.31-1.20 (m, 4H, Cy-H); ¹³C NMR (126 MHz, CDCl₃): δ=138.3, 138.0, 137.8, 128.5, 128.5, 128.4, 128.3, 128.2, 128.1, 127,9, 127.7, 127.4 (Ar—C, C-6), 94.4 (C-1), 82.0 (Cy-C2), 78.3 (C-3), 75.5 (C-2), 74.9, 73.9 (2 PhCH₂), 73.5 (C-4), 73.2 (PhCH₂), 72.5 (Cy-C1), 69.4-68.1 (m, C5), 29.7, 29.3, 24.3. 24.0 (Cy-H); ¹⁹F NMR (470 MHz, CDCl₃): δ=−72.8 (d, J=6.8 Hz, 3F, CF₃); ESI-MS: m/z: Calculated for C₃₃H₃₇F₃NaO₆ [M+Na]⁺: 609.2 found: 609.3.

Synthesis of compound 30: A suspension of donor-compound 20 (52 mg, 0.068 mmol), acceptor-compound 35 (2.0 mg, 0.034 mmol) and activated 4 Å molecular sieves (0.5 g) in dry DCM (2 mL) was stirred at room temperature for 3 hours. In a second flask a suspension of dimethyl(methylthio)sulfonium triflate (DMTST, 44 mg, 0.17 mmol) and activated 4 Å molecular sieves (0.2 g) in DCM (2 mL) was stirred at room temperature for 3 hours and then added via syringe to the first flask. The reaction was stirred at room temperature for 18 hours, filtered through celite and the celite was washed with DCM (50 mL). The filtrate was washed with saturated aqueous NaHCO₃ (20 mL), dried over Na₂SO₄, filtered and concentrated. The crude product was purified by flash chromatography (petroleum ether/EtOAc, gradient 0-50%) to obtain compound 36 (30 mg, 68%). [α]_{D}²² −52.6 (c 0.20, CHCl₃); ¹H NMR (500 MHz, CDCl3): δ=8.14 (d, J=7.2 Hz, 2H, Ar—H), 8.05 (d, J=7.2 Hz, 2H, Ar—H), 7.55 (t, J=7.4 Hz, 2H, Ar—H), 7.44 (td, J=7.7, 15.1 Hz, 4H, Ar—H), 7.31-7.14 (m, 25H, Ar—H), 5.86 (d, J=3.2 Hz, 1H, Gal-H4), 5.52 (t, J=8.9 Hz, 1H, Gal-H2), 5.12 (A of AB, J=12.1 Hz, 1H, PhCH₂), 5.06 (B of AB, J=12.1 Hz, 1H, PhCH₂), 5.01 (d, J=3.3 Hz, 1H, Fuc-H1), 4.89 (m, 1H, Fuc-H5), 4.11 (A′ of A′B′, J=10.8 Hz, 1H, PhCH₂), 4.69 (A″ of A″B″J=12.5 Hz, 1H, PhCH₂), 4.62 (A′″ of A′″B′″, J=11.4 Hz, 1H, PhCH₂), 4.58 (d, J=7.8 Hz, 1H, Gal-H1), 4.57 (B″ of A″B″, J=12.5 Hz, 1H, PhCH₂), 4.49 (B′ of A′B′, J=10.8 Hz, 1H, PhCH₂), 4.43 (A″″ of A″″B″″, J=12.0 Hz, 1H, PhCH₂), 4.41 (B′″ of A′″B′″, J=12.9 Hz, 1H, PhCH₂), 4.37 (B″″ of A″″B″″, J=12.0 Hz, 1H, PhCH₂), 4.11 (dd, J=5.0, 7.6 Hz, 1H, Lac-H2), 4.06 (br s, 1H, Fuc-H4), 4.02 (dd, J=3.4, 10.0 Hz, 1H, Fuc-H2), 3.97 (dd, J=2.7, 10.0 Hz, 1H, Fuc-H3), 3.81 (dd, J=3.2, 9.9 Hz, 1H, Gal-H3), 3.76 (t, J=6.1 Hz, 1H, Gal-H5), 3.64-3.53 (m, 4H, Gal-H6, Cy-H), 1.99-0.38 (m, 21H, Cy-H, Lac-H3); ¹³C NMR (126 MHz, CDCl₃): δ=172.6 (Lac-C1), 166.1, 164.6 (Bz-CO), 138.7, 138.6, 138.4, 138.0, 135.7, 133.3, 133.2, 130.2, 130.1, 130.1, 129.8, 128.7, 128.7, 128.6, 128.6, 128.5, 128.4, 128.4, 128.4, 128.2, 128.2, 128.1 127.8, 127.8, 127.6, 127.6, 127.2 (Ar—C, Fuc-C6), 100.7 (Gal-C1), 94.1 (Fuc-C1), 80.5 (Cy-C), 78.8 (Fuc-C3), 78.4 (Lac-C2), 78.1 (Gal-C3), 75.9 (Fuc-C2), 75.3 (Cy-C), 75.0 (PhCH₂), 74.2 (Fuc-C4), 73.8, 73.4, 73.4 (3 PhCH₂), 73.1 (Gal-C2), 70,3 (Gal-C4), 69.1 (Gal-C6), 68.9 (Gal-C5), 69.1-68.1 (m, Fuc-C5), 66.7 (PhCH₂), 40.6, 33.5, 33.4, 32.9, 30.3, 29.8, 28.7, 28.6, 26.3, 25.9, 25.7, 24.0, 23.2 (Cy-C, Lac-C3); ¹⁹F NMR (470 MHz, CDCl₃): δ=−72.4 (d, J=6.9 Hz, 3F, CF₃); ESI-MS: m/z: Calculated for C₇₆H₈₁F₃NaO₁₅ [M+Na]⁺: 1313.5, found: 1313.6.

Synthesis of compound 37: A suspension of compound 36 (28 mg, 0.022 mmol) and Pd(OH)₂/C (50 mg, 10% Pd) THF (5 mL) was hydrogenated (1 bar H₂) for 2 hours at room temperature. The reaction mixture was filtered through a double layer of filter paper and concentrated in vacua. The crude residue was dissolved in dry MeOH (0.2 mL) and 1M aqueous LiOH (0.43 mL, 0.43 mmol) was added. The reaction was heated via microwave irradiation to 60° C. for 10 h, then diluted with MeOH (5 mL) and neutralized with Amberlite IR120 resin (H⁺ form). The resin was filtered off and the filtrate was concentrated in vacua. The crude residue was dissolved in water (1 mL) and filtered through a short column of Dowex 50 (Na⁺ form) ion exchange resin. The filtrate was concentrated and the crude product was purified by reversed phase column chromatography (C18, H₂O/MeOH, gradient 0-100%) to yield compound 37, which was microfiltered and lyophilized from water (9.8 mg, 69%, 2 steps). ¹H NMR (500 MHz, D₂O): δ=5.19 (d, J=3.7 Hz, Fuc-H1), 5.10 (q, J=6.9 Hz, 1H, Fuc-H5), 4.47 (d, J=7.9 Hz, 1H, Gal-H1), 4.30 (d, J=2.9 Hz, 1H, Fuc-H4), 4.07 (dd, J=3.2, 9.2 Hz, 1H, Lac-H2), 3.98-3.93 (m, 2H, Fuc-H3, Gal-H4), 3.88 (dd, J=3.7, 10.3 Hz, 1H Fuc-H2), 3.78-3.70 (m, 3H, Gal-H6, Cy-H), 3.65-3.52 (m, 3H, Gal-H5, Cy-H, Gal-H2), 3.41 (dd, J=3.1, 9.6 Hz, 1H, Gal-H3), 2.17-2.07 (m, 2H, Cy-H), 1.81 (m, 1H, Cy-H), 1.77-1.53 (m, 9H, Lac-H3, Cy-H), 1.37-1.13 (m, 7H, Cy-H), 1.07-0.87 (m, 2H, Cy-H); ¹³C NMR (126 MHz, CDCl₃): δ=181.9 (Lac-C1), 100.7 (Gal-C1), 95.5 (Fuc-C1), 82.7 (Gal-C3), 79.2 (Cy-C), 78.8 (Lac-C2), 77.2 (Cy-C), 74.3 (Gal-C5), 70.0 (Gal-C2), 68.5 (Fuc-C3), 68.7-67.8 (m, Fuc-C5), 67.5 (Fuc-C2), 67.2 (Fuc-C4), 66.4 (Gal-C4), 61.6 (Gal-C6), 41.0, 33.5. 33.2, 31.9, 30.1, 29.1, 26.1, 25.9, 25.7, 23.2, 23.1 (Cy-C, Lac-C3); ¹⁹F NMR (470 MHz, CDCl₃): δ=−72.6 (d, J=6.8 Hz, 3F, CF₃); HR-MS: m/z: Calculated for C₂₂H₄₂F₃Na₂O₁₃ [M+Na]⁺: 677.2373, found: 677.2371.

### Example 5

### Synthesis of Compound 38

Synthesis of compound 38: A suspension of compound 36 (12 mg, 0.0093 mmol) and Pd(OH)₂/C (20 mg, 10% Pd) in THF (4 mL) was hydrogenated (1 bar H₂) for 2 hours at room temperature. The reaction mixture was filtered through a double layer of filter paper and concentrated in vacuo. The crude residue was dissolved in thy MeOH (0.2 mL) and 1M NaOMe (30 mL, 0.030 mmol) was added. The reaction was stirred at room temperature for 2 hours, then diluted with MeOH (5 mL) and neutralized with Arnberlite IR120 resin (H⁺ form). The resin was filtered off and the filtrate was concentrated in vacua. The crude residue was dissolved in water (1 mL) and filtered through a short column of Dowex 50 (Na⁺ form) ion exchange resin. The filtrate was concentrated and the crude product was purified by reversed phase column chromatography (C18, H₂O/MeCN, gradient 0-100%) to give compound 38, which was microfiltered and lyophilized from water (2.7 mg, 40%, 2 steps). ¹H NMR (500 MHz, D₂O): δ=8.16 (d, J=7.3 Hz, 2H, Ar—H), 7.73 (t, J=7.5 Hz, 1H, Ar—H), 7.59 (t, J=7.8 Hz, 2H, Ar—H), 5.22 (t, J=8.8 Hz, 1H, Gal-H2), 5.16 (d, J=3.8 Hz, 1H, Fuc-H1), 5.10 (q, J=7.2 Hz, 1H, Fuc-H5), 4.89 (d, J==8.1 Hz, 1H, Gal-H1), 4.34 (d, J=3.0 Hz, 1H, Fuc-H4), 4.02 (d, J=2.9 Hz, 1H, Gal-H4), 3.97 (dd, J=3.3, 10.3 Hz, 1H, Fuc-H3), 3.91-3.84 (m, 2H, Fuc-H2, Lac-H2), 3.92-3.78 (m, 4H, Gal-H3, Gal-H5, Gal-H6), 3.69 (m, 1H, Cy-H), 3.48 (m, 1H, Cy-H), 2.10L98 (m, 2H, Cy-H), 1.63 (m, 1H, Cy-H), 1.58-1.48 (m, 2H, Cy-H), 1.45 (m, 1H, Lac-H3), 1.40-0.93 (m, 10H, Lac-H3′, Cy-H), 0.87 (m, 1H, Cy-H), 0.78-0.61 (m, 2H, Cy-H), 0.54 (m, 1H, Cy-H), 0.40 (m, 1H, Cy-H); ¹³C NMR (126 MHz, CDCl₃): δ=167.9 (Lac-C1), 134.1, 129.9, 129.0, 128.9 (Ar—C), 99.2 (Gal-C1), 95.2 (Fuc-C1), 81.2 (Gal-C3), 80.0 (Cy-C), 79.1 (Lac-C2), 76.6 (Cy-C), 74.5 (Gal-C5), 72.2 (Gal-C2), 68.6 (Fuc-F3), 68.7-67.8 (m, Fuc-C5), 67.5 (Fuc-C2), 67.2 (Fuc-C4), 66.4 (Gal-C4), 61.6 (Gal-C6), 41.7 (Lac-C3), 33.6, 32.9, 31.4, 30.3, 28.7, 25.7, 25.2, 25.0, 23.0 (Cy-C); ¹⁹F NMR. (470 MHz, CDCl₃): δ=−72.5 (d, J=6.8 Hz, 3F, CF₃); HR-MS: m/z: Calculated for C₃₄H₄₆F₃Na₂O₁₄ [M+Na]⁺: 781.2635, found: 781.2635.

### Example 6

### Synthesis of Compound 41

Synthesis of compound 39: A suspension of donor 11 (110 mg, 0.174 mmol), acceptor 17 (59 mg, 0.348 mmol) and activated 4 ∈ molecular sieves (0.5 g) in DCM (5 mL) was stirred at room temperature for 1 hour then TBDMS-OTf (14 μL) was added at 0° C. The reaction was allowed to waiting up to room temperature and stirred for 18 hours, then quenched with Et3N (0.3 mL). The mixture was filtered through celite, and the filtrate was concentrated in vacuo. The crude residue was purified by flash chromatography (petroleum ether/Et₂O, gradient 0-20%) to isolate the glycosylation product as a mixture of α/β-anomers (67 mg, 60%). The mixture of anomers was dissolved in MeCN (0.2 mL) and H₂O (3.3 μL) and PdCl₂MeCN₂ (8 mg, 0.031 mmol) were added. The mixture was stirred under air atmosphere at 60° C. for 18 hours then concentrated and purified by flash chromatography (petroleum ether/EtOAc, gradient 0-25%) to obtain product compound 39 (19 mg, 18%, 2 steps). [α]_{D}²² −40.1 (c 0.24, CHCl₃); ¹H NMR (500 MHz, CDCl₃): δ =7.38-7.26 (m, 15H, Ar—H), 5.19 (d, J=3.6 Hz, 1H, H-1), 4.91 (A of AB, J=10.8 Hz, 1H, PhCH₂), 4.82 (A′ of A′B′, J=11.8 Hz, 1H, PhCH₂), 4.80 (A″ of A″B″, J=11.7 Hz, 1H, PhCH₂), 4.74 (B′ of J=A′B′, 11.8 Hz, 1H, PhCH₂), 4.71 (B″ of A″B″, J=11.7 Hz, 1H, PhCH₂), 4.66 (B of AB, J=10.8 Hz, 1H, PhCH₂), 4.13 (dd, J=3.5, 10.2 Hz, 1H, H-2), 4.43 (q, J=6.4 Hz, 1H, H-5), 4.13-4.15 (m, 2H, H-4, H-2), 3.99 (dd, J=2.7, 10.2 Hz, 1H, H-3), 3.40 (m, 1H, CyMe-H1), 3.01 (dd, J=3.9, 10.2 Hz, 1H, CyMe-H2), 2.00 (m, 111, CyMe-H6a), 1.71-1.52 (m, 3H, CyMe-H3, CyMe-H4a, CyMe-H5a), 1.33-1.16 (m, 2H, CyMe-H5b, CyMe-H6b), 1.05 (d, J=6.4 Hz, 3H, CH₃), 0.99 (m, 1H, CyMe-H4b); ¹³C NMR (126 MHz, CDCl₃): δ=138.2, 137.8, 137.8, 128.4, 128.4, 128.2, 128.2, 128.2, 127.9, 127.7, 127.7, 127.5 (Ar—C, C-6), 97.4 (C-1), 92.0 (Cy-C2), 77.8 (C-3), 75.7 (C-2), 74.9, 74.0 (2 PhCH₂), 73.6 (C-4), 73.1 (PhCH₂), 72.5 (Cy-C1), 69.6 (q, J=31.5 Hz, C-5), 36.1 (CyMe-C3), 33.8 (CyMe-C4), 33.0 (CyMe-C6), 23.2 (CyMe-C5), 18.6 (CyMe-CH₃); ¹⁹F NMR (470 MHz, CDCl₃): δ=−72.7 (d, J=6.6 Hz, 3F, CF₃); ESI-MS: m/z: Calculated for C₃₄H₃₉F₃NaO₆ [M+Na]⁺: 623.3 found: 623.2.

Synthesis of compound 40: A suspension of donor-compound 20 (30.6 mg, 0.0399 mmol), acceptor-compound 39 (12 mg, 0.020 mmol) and activated 4 Å molecular sieves (0.3 g) in dry DCM (2 mL) was stirred at room temperature for 3 hours. In a second flask a suspension of dimethyl(methylthio)sulfonium triflate (DMTST, 25 mg, 0.095 mmol) and activated 4 Å molecular sieves (0.2 g) in DCM (2 mL) was stirred at room temperature for 3 hours then added via syringe to the first flask. The reaction was stirred at room temperature for 18 hours, filtered through celite, and the celite was washed with DCM (50 mL). The filtrate was washed with saturated aqueous NaHCO₃ (20 mL), dried over Na₂SO₄, filtered and concentrated. The crude product was purified by flash chromatography (petroleum ether/EtOAc, gradient 0-50%) to give compound 40 (12 mg, 46%). [α]_{D}²² −39.3 (c 0.16, CHCl₃); ¹H NMR (500 MHz, CDCl₃): δ=8.15 (d, J=7.2 Hz, 2H, Ar—H), 8.05 (m, 2H, Ar—H), 7.55 (m, 2H, Ar—H), 7.44 (m, 4H, Ar—H), 7.33-7.10 (m, 25H, Ar—H), 5.86 (d, J=3.3 Hz, 1H, Gal-H4), 5.53 (t, J=9.0 Hz, 1H, Gal-H2), 5.26 (d, 3.3 Hz, 1H, Fuc-H1), 5.21 (m, 1H, Fuc-H5), 5.09 (m, 2H, PhCH₂), 4.76-4.63 (m, 3H, PhCH₂), 4.58 (d, J=8.2 Hz, 1H, Gal-H1), 4.56-4.35 (m, 5H, PhCH₂), 4.11-4.00 (m, 4H, Lac-H2, Fuc-H2, Fuc-H3, Fuc-H4), 3.83 (dd, J=2.7, 9.6 Hz, 1H, Gal-H3), 3.78 (t, J=6.2 Hz, 1H, Gal-H5), 3.63-3.52 (m, 4H, Gal-H6, CyMe-H1), 3.31 (t, J=9.2 Hz, 1H, CyMe-H2), 1.90 (m, 1H, CyMe-H), 1.50-1.07 (m, 10H, Lac-H3, Cy-H, CyMe-H), 1.04 (d, J=6.6 Hz, 3H CyMe-CH₃) 0.98-0.63 (m, 7H, Cy-H, CyMe-H), 0.53-0.40 (m, 2H, Cy-H, CyMe-H); ¹³C NMR (126 MHz, CDCl₃): δ=172.4 (Lac-C1), 166.2, 164.3 (Bz-CO), 138.4, 138.3, 138.2, 137.8, 135.6, 133.1, 130.1, 130.0, 129.9, 129.7, 128.5, 128.5, 128.4, 128.3, 128.2, 128.2, 128.2, 128.0, 127.9, 127.7, 127.6, 127.6, 127.4, 127.4, 127.1 (Ar—C, Fuc-C6), 100.1 (Gal-C1), 97.3 (Fuc-C1), 82.1 (CyMe-C1), 81.2 (CyMe-C2), 78.6, 78.4 (Fuc-C3, Lac-C2), 78.2 (Gal-C3), 75.5 (Fuc-C2), 74.7 (PbCH₂), 74.1 (2C, Fuc-C4, PhCH₂), 73.6 (PhCH₂), 73.3 (Gal-C5), 72.5 (2C, Gal-C2, PhCH₂), 70.2 (Gal-C4), 68.7 (Gal-C6), 68.9-68.4 (m, Fuc-C5), 66.6 (PhCH₂) 40.4, 38.9, 33.3, 33.2, 33.1, 31.3, 29.7, 26.1, 25.7, 25.5 (CyMe-C, Cy-C, Lac-C3), 19.0 (CyMe-CH₃); ¹⁹F NMR (470 MHz, CDCl₃): δ=−72.1 (d, J=6.7 Hz, 3F, CF₃); ESI-MS: m/z: Calculated for C₇₇H₈₃F₃NaO₁₅ [M+Na]⁺: 1327.6, found: 1327.7.

Synthesis of compound 41: A suspension of compound 40 (10 mg, 0.0076 mmol) and Pd(OH)₂/C (20 mg, 10% Pd) in THF (4 mL) was hydrogenated (1 bar H₂) for 2 h at room temperature. The reaction mixture was filtered through a double layer of filter paper and concentrated in vacuo. The crude residue was dissolved in dry MeOH (0.2 mL) and 1M aqueous LiOH (0.15 mL, 0.15 mmol) was added. The reaction was heated via microwave irradiation to 60° C. for 10 h, then diluted with MeOH (5 mL) and neutralized with Amberlite IR120 resin (H⁺ form). The resin was filtered off and the filtrate was concentrated in vacuo. The residue was dissolved in water (1 mL) and filtered through a short column of Dowex 50 (Na⁺ form) ion exchange resin. The filtrate was concentrated and the crude product was purified by reversed phase column chromatography (C18, H₂O/MeOH, gradient 0-100%) to give compound 41, which was microfiltered and lyophilized from water (2 mg, 40%, 2 steps). ¹H NMR (500 MHz, D₂O): δ=5.38-5.32 (m, 2H, Fuc-H1, Fuc-H5), 4.44 (d, J=8.1 Hz, 1H, Gal-H1), 4.30 (d, J=2.3 Hz, 1H, Fuc-H4), 4.00-3.94 (m, 2H, Lac-H2, Fuc-H3), 3.93-3.87 (m, 2H, Gal-H4, Fuc-H2), 3.77-3.72 (m, 2H, Gal-H6), 3.69 (m, 1H, CyMe-H1), 3.62-3.55 (m, 2H, Gal-H5, Gal-H2), 3.40-3.31 (m, 2H, CyMe-H2, Gal-H3), 2.11 (m, 1H, CyMe-H), 1.81 (m, 1H, CyMe-H or Cy-H), 1.73-1.51 (m, 10H, Lac-H3, CyMe-H, Cy-H), 1.41-1.15 (m, 6H, CyMe-H, Cy-H), 1.13 (d, J=6.2 Hz, 3H, CyMe-CH₃), 1.04-0.84 (m, 2H, CyMe-H, Cy-H); ¹³C NMR (126 MHz, CDCl₃): δ=182.8 (Lac-C1), 101.1 (Gal-C1), 98.6 (Fuc-C1), 83.3 (CyMe-C2), 82.9 (Gal-C3), 80.1 (CyMe-C1), 79.2 (Lac-C2), 74.2 (Gal-C5), 69.6 (Gal-C2), 69.0-68.1 (m, Fuc-C5), 68.0 (Gal-C4), 67.7 (Fuc-C3), 67.3 (Fuc-C4), 66.3 (Fuc-C2), 61.8 (Gal-C6), 41.2, 38.9, 33.6, 33.3, 33.1, 31.9, 30.7, 26.2, 25.9, 25.7, 22.7 (CyMe-C, Cy-C, Lac-C3), 18.3 (CyMe-CH₃); ¹⁹F NMR (470 MHz, CDCl₃): δ=−72.3 (d, J=6.9 Hz, 3F, CF₃); HR-MS: m/z: Calculated for C₂₈H₄₄F₃Na₂O₁₃ [M+Na]⁺: 691.2529, found: 691.2529.

### Example 7

### Synthesis of Compound 42

Synthesis of compound 42: A suspension of compound 40 (14 mg, 0.0113 mmol) and Pd(OH)₂/C (20 mg, 10% Pd) in THF (4 mL) was hydrogenated (1 bar H₂) for 2 hours at room temperature. The reaction mixture was filtered through a double layer of filter paper and concentrated in vacuo. The crude residue was dissolved in H₂O/dioxane (1:1, 1 mL) and 1M NaOH (28 μL) was added. The reaction was stirred at room temperature for 18 hours, then lyophilized. The crude product was purified by reversed phase column chromatography (C18, H₂O/MeCN, gradient 0-50%) to give compound 42, which was microfiltered and lyophilized from water (3 mg, 34%, 2 steps). ¹H NMR (500 MHz, D₂O): δ=8.15 (d, J=7.2 Hz, 2H, Ar—H), 7.72 (t, J=7.3 Hz, 1H, Ar—H), 7.58 (t, J=7.5 Hz, 2H, Ar—H), 5.40 (app q, J=6.6 Hz, 1H, Fuc-H5), 5.31 (d, J=3.6 Hz, 1H, Fuc-H1), 5.27 (t, J=8.8 Hz, 1H, Gal-H2), 4.86 (d, J=8.2 Hz, 1H, Gal-H1), 4.32 (d, J=2.1 Hz, Fuc-H4), 4.01 (br s, 1H, Gal-H4), 3.97 (dd, J=2.9, 10.4 Hz, 1H, Fuc-H3), 3.93-3.84 (m, 2H, Fuc-H2, Lac-H2), 3.84-3.70 (m, 4H, Gal-H3, Gal-H5, Gal-H6), 3.65 (m, 1H, CyMe-H1), 3.24 (t, J=9.5 Hz, 1H, CyMe-H2), 2.04 (m, 1H, CyMe-H), 1.65-1.15 (m, 11H, Cy-H, CyMe-H, Lac-H3), 1.07 (d, J=6.0 Hz, 3H, CyMe-CH₃), 1.05-0.80 (m, 4H, Cy-H, CyMe-H), 0.73-0.59 (m, 2H, Cy-H, CyMe-H), 0.52 (m, 1H, Cy-H or CyMe-H), 0.37 (m, 1H, Cy-H or CyMe-H); ¹³C NMR (126 MHz, CDCl₃): δ=172.8 (PhCO), 167.7 (Lac-C1), 134.1, 129.9, 129.0, 128.9 (Ar—C), 99.7 (Gal-C1), 98.5(Fuc-C1), 82.8 (Gal-C3), 81.4, 81.3 (CyMe-C, Lac-C2), 79.0 (CyMe-C), 74.4 (Gal-C5), 71.9 (Gal-C2), 68.8-67.9 (m, Fuc-C5), 68.1 (Fuc-F3), 67.8 (Fuc-C2), 67.4 (Fuc-C4), 66.4 (Gal-C4), 61.7 (Gal-C6), 41.7 (Lac-C3), 38.7, 33.6, 32.9, 32.9, 31.4, 31.1, 25.7, 25.2, 24.9, 22.4 (CyMe-C, Cy-C), 18.2 (CyMe-CH₃); ¹⁹F NMR (470 MHz, CDCl₃): δ=−72.4 (d, J=6.8 Hz, 3F, CF₃); HR-MS: m/z: Calculated for C₃₅H₄₈F₃Na₂O₁₄.

### Example 8

### Synthesis of Compound 51

Synthesis of compound 45: A suspension of 43 (G. Hirai et al., J. Am. Chem, Soc., 129, 15420-15421 (2007)) (2.00 g, 5.34 mmol) and n-Bu₂SnO (1.99 g, 8.00 mmol) in dry MeOH (20 mL) was heated to reflux at 70° C. under argon for 6 h. The reaction was allowed to cool to rt, and dry toluene (150 mL) was added. The solvents were removed in vacuo and the residue was dried in high vacuum. for 3 h, then a solution of 19 (M. Qabar et al., Tetrahedron Lett., 39, 5895-5898 (1998)) (4.20 g, 10.7 mmol) in dry DME (20 mL) was added. To the resulting suspension anhydrous CsF (2.43 g, 16.0 mmol, dried in high vacuum at 120° C. for 3 h) was added and the turbid mixture was stirred under argon at rt for 18 h. The reaction was quenched by addition of 1M K₂HPO₄ containing 10% KF (20 mL) at 0° C. The mixture was stirred at rt for 30 min, then extracted with DCM (3×100 mL). The combined organic phases were washed with satd. aq. NaHCO₃ (50 mL), dried over Na₂SO₄, filtered and concentrated in vacuo. The crude product was purified by flash chromatography (toluene/EtOAc, gradient 0-20%) to yield lactone 44 (990 mg, 36%, 2 steps) and benzylester 45 (730 mg, 22%, 2 steps).

Compound 44: [α]_{D}²² −50 (c 1.0, CHCl₃); ¹H NMR (500 MHz, CDCl₃): δ=7.54-7.48 (m, 2H, Ar—H), 7.40-7.32 (m, 3H, Ar—H), 7.14-7.07 (m, 2H, Ar—H), 6.85-6.79 (m, 2H, Ar—H), 5.61 (s, 1H, PhCH), 5.02 (d, J=7.8 Hz, 1H, H-1), 4.89 (dd, J=7.8, 10.2 Hz, 1H, H-2), 4.62, (dd, J=4.5, 9.2 Hz, 1H, Lac-H2), 4.44-4.36 (m, 2H, H-4, H-6), 4.13 (dd, J=1.7, 12.5 Hz, 1H, H-6′), 3.84 (dd, J=3.2, 10.2 Hz, 1H, H-3), 3.78 (s, 3H, OCH₃), 3.63 (d, J=0.8 Hz, 1H, H-5), 1.85-1.62 (m, 7H, Cy-H, Lac-H-3), 1.62-1.52 (m, 1H, Cy-H), 1.31-1.11 (m, 3H, Cy-H), 1.06-0.85 (m, 2H, Cy-H); ¹³C NMR (126 MHz, CDCl₃): δ=171.0 (Lac-C1), 129.4, 129.2, 128.4, 128.4, 126.5, 119.9, 114.6, (Ar—C), 101.3 (PhCH), 100.3 (C-1), 73.9, 73.8 (C-2, C-4), 72.5 (Lac-C2), 71.1 (C-3), 69.3 (C-6), 67.1 (C-5), 55.8 (OCH₃), 38.6 (Lac-C3), 34.1, 33.7, 32.1, 26.6, 26.4, 26.1 (Cy-C); ESI-MS: m/z: Calcd for C₂₉H₃₄NaO₈ [M+Na]⁺: 533.2, found: 533.2.

Compound 45: [α]_{D}²² −37.9 (c 0.66, CHCl₃); ¹H NMR (500 MHz, CDCl₃): δ=7.58-7.45 (m, 2H, Ar—H), 7.42-7.28 (m, 8H, Ar—H), 7.09-6.97 (m, 2H, Ar—H), 6.84-6.69 (m, 2H, Ar—H), 5.43 (s, 1H, PhCH), 5.14-5.06 (m, 2H, PhCH₂), 4.72 (d, J=7.7 Hz, 1H, H-1), 4.55, (dd, J=4.4, 9.0 Hz, 1H, Lac-H2), 4.27, (d, J=12.9 Hz, 1H, H-6), 4.25, (d, J=3.4 Hz, 1H, H-4), 4.19 (dd, 7.8, 9.7 Hz, 1H, H-2), 3.96 (dd, J=1.1, 12.2 Hz, 1H, H-6′), 3.76 (s, 3H, OCH₃), 3.50 (dd, J=3.4, 9.8 Hz, 1H, H-3), 3.37 (br s, 1H, H-5), 2.55 (br s, 1H, OH), 1.85-1.48 (m, 7H, Cy-H, Lac-H3), 1.24-1.08 (m, 3H, Cy-H), 0.98-0.81 (m, 3H, Cy-H); ¹³C NMR (126 MHz, CDCl₃); δ=173.9 (Lac-C1), 155.7, 151.4, 138.0, 135.8, 128.9, 128.7, 128.7, 128.6, 128.2, 127.1, 126.6, 119.3, 114.6, (Ar—C), 103.0 (PhCH), 101.0 (C-1), 79.7 (C-3), 78.0 (Lac-C2), 75.1 (C-4), 70.8 (C-2), 69.1 (C-6), 67.1 (C-5), 66.7 (PhCH₂), 55.8 (OCH₃), 40.7 (Lac-C3), 33.9, 33.9, 32.7, 26.6, 26.4, 26.3 (Cy-C); ESI-MS: m/z: Calcd for C₃₆H₄₂NaO₉ [M+Na]⁺: 641.3, found: 641.2.

Synthesis of compound 47: A suspension of donor 11 (700 mg, 1.11 mmol), acceptor 46 (G. Hirai et al., J, Am. Chem. Soc., 129, 15420-15421 (2007)) (400 mg, 1.55 mmol) and activated 4 Å molecular sieves (2 g) in dry DCM/1,4-dioxane (1:2, 6 mL) was stirred at rt for 1 h under argon, then TBSOTf (51 μL) was added at 0° C. The reaction was allowed to warm up to rt and stirred for 18 h, then quenched with Et₃N (0.5 mL). The mixture was filtered through celite, and the filtrate was concentrated in vacuo. The crude residue was purified by flash chromatography (petroleum ether/Et₂O, gradient 0-20%) to isolate the glycosylation product as a mixture of α/β-anomers (572 mg, 71%). The mixture of anomers was dissolved in MeCN (3 mL) and HF-pyridine (0.5 mL) was added at rt. The mixture was stirred under argon for 2 d then DCM (5 mL) was added followed by an addition of said. aq. NaHCO₃ (5 mL) at 0° C. The resulted suspension was stirred at rt for 30 min then washed with DCM (2×100 mL). The combined organic phases were washed with said, aq. NaHCO₃, dried over Na₂SO₄ and concentrated in vacuo. The crude residue was purified by flash chromatography (petroleum ether/EtOAc, gradient 0-30%) to obtain product 47 (191 mg, 28%, 2 steps). [α]_{D}²² −54.7 (c 0.79, CHCl₃); ¹H NMR (500 MHz, CDCl₃): δ=7.39-7.26 (m, 15H, Ar—H), 5.18 (d, J=3.5 Hz, 1H, H-1), 4.91 (A of AB, J=10.8 Hz, 1H, PhCH₂), 4.81 (A′ of A′ B′, J=11.7 Hz, 1H, PhCH₂), 4.80 (A″ of A″B″, J=11.8 Hz, PhCH₂), 4.75 (B″ of A″B″, J=11.8 Hz, 1H, PhCH₂), 4.70 (B′ of A′B′, J=11.7 Hz, 1H, PhCH₂), 4.66 (B of AB, J=10.8 Hz, 1H, PhCH₂), 4.13 (dd, J=3.5, 10.2 Hz, 1H, H-2), 4.41 (q, J=6.4 Hz, 1H, H-5), 4.20-4.14 (m, 2H, H-4, H-2), 3.98 (dd, J=2.5, 10.2 Hz, 1H, H-3), 3.88 (d, J=2.7 Hz, 1H, OH), 3.40 (m, 1H, CyMe-H1), 3.10 (m, 1H, CyMe-H2), 2.03-1.89 (m, 2H, CyMe-H6, CH₂CH₃), 1.82 (m, 1H, CyMe-H4), 1.68 (m, 1H, CyMe-H5), 1.36-1.04 (m, 4H, CyMe-H3, CyMe-H5′, CyMe-H6′, CH₂CH₃), 0.88 (m, 1H, CyMe-H4′), 0.79 (t, J=7.4 Hz, 3H, CH₂CH₃); ¹³C NMR (126 MHz, CDCl₃): δ=138.3, 137.9, 137.9, 128.6, 128.5, 128.4, 128.3, 128.3, 128.0, 127,9, 127.8, 127.6 (Ar—C, C-6), 97.2 (C-1), 92.2 (Cy-C2), 77.9 (C-3), 75.7 (C-2), 75.1, 74.3 (2 PhCH₂), 73.6 (C-4), 73.1 (PhCH₂), 72.7 (Cy-C1), 69.8 (q, J=31.0 Hz, C-5), 42.3 (CyMe-C3), 33.0 (CyMe-C6), 29.8 (CyMe-C4), 24.4 (CH₂CH₃), 24.3 (CyMe-C5), 11.0 (CH₂CH₃); ¹⁹F NMR (470 MHz, CDCl₃): δ=−72.7 (d, J=6.6 Hz, 3F, CF₃); ESI-MS: m/z: Calcd for C₃₅H₄₁F₃NaO₆ [M+N]⁺: 637.3 found: 637.2.

Synthesis of compound 48: To a solution of 44 (227 mg, 0.44 mmol) and 3,3-difluororazetidine hydrochloride (114 mg, 0.88 mmol) in DCM (3 ml) Et₃N (0.37 mL, 2.66 mmol) was added. The reaction was stirred at rt for 18 h then purified directly by a short flash chromatography (toluene/EtOAc, gradient 0-80%) to isolate the amide intermediate (169 mg, 63%). To the solution of the intermediate (160 mg, 0.26 mmol) in pyridine (2 mL) BzCl (61 μL) was added at 0° C. The mixture was stirred at it for 4 h then diluted with toluene (20 mL) and concentrated in vacuo. The crude residue was purified by flash chromatography (toluene/EtOAc, gradient 0-80%) to yield 48 (123 mg, 66%; 41% after 2 steps). [α]_{D}²² −26.6 (c 0.50, CHCl₃); ¹H NMR (500 MHz, CDCl₃): δ=8.10-8.02 (m, 2H, Ar—H), 7.62-7.53 (m, 3H, Ar—H), 7.52-7.36 (m, 5H, Ar—H), 7.03-6.92 (m, 2H, Ar—H), 6.83-6.72 (m, 2H, Ar—H), 5.79 (dd, J=8.1, 10.0 Hz, 1H, H-2), 5.51 (s, 1H, PhCH), 5.09 (d, J=8.0 Hz, 1H, H-1), 4.73 (m, 1H, CF₂CH₂), 4.42-4.27 (m, 3H, H-4, H-6, CF₂CH₂), 4.11 (dd, J==1.6, 12.4 Hz, 1H, H-6′), 4.06-3.95 (m, 2H, CF₂CH₂, Lac-H2), 3.80-3.73 (m, 4H, H-3, OCH₃), 3.67 (m, 1H, CF₂CH₂), 3.58 (br s, 1H, H-5), 1.61-1.14 (m, 8H, Cy-H, Lac-H3), 1,00-0.83 (m, 2H, Cy-H), 0.79-0.56 (m, 3H, Cy-H); ¹³C NMR (126 MHz, CDCl₃): δ=171.8 (t, J=3.7 Hz, Lac-C1), 165.2 (PhCO,), 155.7, 151.3, 137.2, 133.3, 129.8, 129.7, 129.7, 128.6, 128.5, 126.6, 119.5 (Ar—C), 115.7 (t, J=271 Hz, CF₂), 114.6 (Ar—C), 102.1 (PhCH), 101.1 (C-1), 80.0 (C-3), 78.8 (Lac-C2), 73.0 (C-4), 70.0 (C-2), 69.1 (C-6), 66.4 (C-5), 63.4 (t, J==28.3 Hz, CF₂CH₂), 60.1 (t, J=28.6 Hz, CF₂CH₂), 55.6 (OCH₃), 40.5 (Lac-C3), 33.7, 33.2, 32.5, 26.1, 25.7, 25.6 (Cy-C); ¹⁹F NMR (470 MHz, CDCl₃): δ=−100.36-−102,82 (m, 2F, CF₂); ESI-MS: m/z: Calcd for C₃₉H₄₃F₂NNaO₉ [M+N]⁺: 730.3 found: 730.3.

Synthesis of compound 49: To a solution of 48 (110 mg, 0.155 mmol) in MeCN/H₂O (1:1, 2 ml) CAN (426 mg, 0.78 mmol) was added at −20° C. The reaction was stirred at rt for 2 h then brine (5 mL) was added and the resulting mixture was extracted with EtOAc (3×30 mL). The combined organic phases were dried over Na₂SO₄ and concentrated in vacua. The crude residue was purified by flash chromatography (toluene/acetone, gradient 10-50%) to isolate the intermediate (50 mg, 53%). To a solution of the intermediate (50 mg, 0.0811 mmol) in dry DCM (1 mL) were added Cl₃CCN (0.41 μL) and DBU (3.6 μL, 0.024 mmol). The reaction was stirred under argon for 3 h at rt, then the mixture was purified directly by a short flash chromatography (petroleum ether/EtOAc, gradient 0-50%) to give 49 (38.6 mg, 63%, 33% after 2 steps). ¹H NMR (500 MHz, CDCl₃): δ=8.60 (s, 1H, NH), 8.05-8.02 (m, 2H, Ar—H), 7.61-7.50 (m, 3H, Ar—H), 7.47-7.35 (m, 5H, Ar—H), 7.03-6.92 (m, 2H, Ar—H), 6.83-6.72 (m, 2H, Ar—H), 6.75 (d, J=3.45 Hz, 1H, H-1), 5.55 (dd, J=3.5, 10.4 Hz, 1H, H-2), 5.53 (s, 1H, PhCH), 4.70 (m, 1H, CF₂CH₂), 4.48 (d, J=2.8 Hz, 1H, H-4), 4.38-4.25 (m, 2H, H-6, CF₂CH₂), 4.21 (dd, J=3.3, 10.4 Hz, 1H, H-3), 4.15-4.05 (m, 3H, CF₂CH₂, Lac-H2, H-6′), 3.98 (br s, 1H, H-5), 3.83 (m, 1H, CF₂CH₂), 1.67-1.28, (m, 8H, Cy-H, Lac-H3), 1.05-0.92 (m, 2H, Cy-H), 0.92-0.65 (m, 3H, Cy-H); ¹³C NMR (126 MHz, CDCl₃): 8=172.1 (t, J=3.4 Hz, Lac-C1), 165.5, 160.5 (CNH, PhCO), 137.3, 133.6, 130.0, 129.8, 129.5, 128.7, 128.6 126.5 (Ar—C), 115.8 (m, CF₂), 102.1 (PhGH), 95.0 (C-1), 91.3 (CCl₃), 78.9 (Lac-C2), 76.1 (C-3), 73.6 (C-4), 69.2 (C-6), 68.2 (C-2), 65.2 (C-5), 63.5 (t, J=28.5 Hz, CF₂CH₂), 60.3 (t, J=28.9 Hz, CF₂CH₂), 40.7 (Lac-C3), 33.9, 33.4, 32.6, 26.3, 26.0, 26.0 (Cy-C); ¹⁹F NMR (470 MHz, CDCl₃): δ=−100.40-−102.56 (m, 2F, CF₂); ESI-MS: m/z: Calcd for C₃₄H₃₇Cl₃F₂N₂NaO₈ [M+Na]⁺: 767.1 found: 767.2.

Synthesis of compound 50: A suspension of donor 49 (37 mg, 0.051 mmol), acceptor 47 (14 mg, 0.023 mmol) and activated 4 Å molecular sieves (0.3 g) in dry DCM (1 mL) was stirred at rt for 1 h under argon. The suspension was cooled to −20° C. and a solution of TMSOTf (0.87 μL, 4.5 μmol) in dry DCM (50 μL) was added. The reaction was allowed to gradually warm up to 0° C. (3 h), then quenched with Et₃N (0.1 mL). The mixture was filtered through celite, and the filtrate was concentrated in vacuo. The crude residue was purified by flash chromatography (toluene/acetone, gradient 5-30%) to isolate product 50 (13.2 mg, 48%). [α]_{D}²² −29.8 (c 0.33, CHCl₃); ¹H NMR (500 MHz, CDCl₃): δ=8.06 (d, J=7.2 Hz, 2H, Ar—H), 7.61-7.54 (m, 3H, Ar—H), 7.45 (t, J=7.7 Hz, 2H, Ar—H), 7.43-7.14 (m, 18H, Ar—H), 5.60 (s, 1H, PhCH), 5.48 (t, J=8.7 Hz, 1H, Gal-H2), 5.35 (q, J=6.4 Hz, 1H, Fuc-H5), 5.07 (d, J=2.1 Hz, 1H, Fuc-H1), 5.02 (m, 1H, CH₂CF₂), 4.82 (A of AB, J=11.8 Hz, 1H, PhCH₂), 4.70 (d, J=8.3 Hz, 1H, Gal-H1), 4.65 (B of AB, J=10.8 Hz, 1H, PhCH₂), 4.60 (A′ of A′B′, J=11.3 Hz, 1H, PhCH₂), 4.57-4.46 (m, 2H, CH₂CF₂, B′ of PhCH₂), 4.41 (d, J==11.5 Hz, 1H, Gal-H6), 4.33 (d, J=3.5 Hz, 1H, Gal-H4), 4.27 (A″ of A″B″, J=10.8 Hz, 1H, PhCH₂), 4.17-3.92 (m, 7H, Gal-H6′, CH₂CF₂, B″ of PhCH₂, Lac-H2, Fuc-H2, Fuc-H3, Fuc-H4), 3.83 (m, 1H, CH₂CF₂), 3.71 (m, 1H, Gal-H3), 3.61 (m, 1H, CyEt-H1), 3.45 (br s, 1H, Gal-H5), 3.38 (t, J=9.6 Hz, 1H, CyEt-H2), 1.98 (m, 1H, CyEt-H), 1.89 (m, 1H, CH₂CH₃), 1.74-1.57 (m, 2H, CyEt-H), 1.54-1.08 (m, 12H, Lac-H3, Cy-H, CyEt-H, CH₂CH₃), 0.99-0.54 (m, 9H, Cy-H, CyEt-H, CH₂CH₃); ¹³C NMR (126 MHz, CDCl₃): δ=171.9 (Lac-C1), 164.6 (PhCO), 139.1, 138.9, 138.5, 137.4, 133.2, 130.2, 129.8, 129.3, 129.2, 12.8.8, 128.6, 128.5, 128.4, 128.4, 128.3, 128.1, 127.9, 127,8, 127,6, 127.4, 127.3, 126.2 (Ar—C, Fuc-C6, CF₂), 101.2 (PhCH), 100.8 (Gal-C1), 97.9 (Fuc-C1), 82.6 (CyEt-C1), 81.2 (CyMe-C2), 80.5 (Gal-C3), 79.0 (CyEt-C2), 78.6, 78.5 (Fuc-C3, Lac-C2), 75.2 (Fuc-C2), 74.9, 74.7 (PhCH₂), 74.1 (Fuc-C4), 73.4 (Gal-C4), 71.8 (PhCH₂), 70.3 (Gal-C2), 69.5 (Gal-C6), 68.7 Fuc-C5), 66.7 (Gal-C5), 63.9 (CH₂CF₂), 60.4 (CH₂CF₂), 45.6, 40.4, 33.8, 33.2, 32.8, 31.5, 28.6, 26.3, 25.8, 25.7, 23.5, 23.4 (CyEt-C, Cy-C, Lac-C3, CH₂CH₃), 10.9 (CH₂CH₃); ¹⁹F NMR (470 MHz, CDCl₃): δ=−71.8 (d, J=6.8 Hz, 3F, CF₃), −100.08-−102.04 (m, 2H, CF₂); ESI-MS: m/z: Calcd for C₆₇H₇₆F₅NNaO₁₃ [M+N]⁺: 1220.5, found: 1220.5.

Synthesis of compound 51: A suspension of 50 (10 mg, 8.3 μmol) and Pd(OH)₂/C (10 mg, 10% Pd) in THF (4 mL) was hydrogenated (1 bar H₂) for 2 h at rt. The reaction mixture was filtered through a double layer of filter paper and concentrated in vacua. The crude residue was purified by flash chromatography (DCM/MeOH, gradient 0-20%) to isolate product 51 (6.1 mg, 87%). [α]_{D}²² −51.5 (c 0.20, MeOH); ¹H NMR (500 MHz, CD₃OD): δ=8.09 (d, J=7.3 Hz, 2H, Ar—H), 7.72 (t, J=7.4 Hz, 1H, Ar—H), 7.51 (t, J=7.7 Hz, 2H, Ar—H), 5.38 (dd J=8.3, 9.5 Hz, Gal-H2), 5.23 (q, J=6.8 Hz, 1H, Fuc-H5), 5.14 (d, J=3.8 Hz, 1H, Fuc-H1), 4.99 (m, 1H, CH₂CF₂), 4.73-4.64 (m, 2H, Gal-H1, CH₂CF₂), 4.40-4.25 (m, 2H, CH₂CF₂), 4.19 (d, J=2.2 Hz, 1H, Fuc-H4), 4.08 (dd, J=3.2, 9.1 Hz, 1H, Lac-H2), 3.96 (d, J=2.3 Hz 1H, Gal-H4), 3.94 (dd, J=3.2, 10.3 Hz, 1H, Fuc-H3), 3.86-3.71 (m, 3H, Fuc-H2, Gal-H6), 3.66 (d, J=3.1, 9.8 Hz 1H, Gal-H3), 3.62 (m, 1H, CyEt-H1), 3.54 (t, J=6.0 Hz 1H, Gal-H5), 3.35 (t, J=8.9 Hz, 1H, CyEt-H2), 1.97 (m, 1H, CyEt-H), 1.86 (m, 1H, CH₂CH₃), 1.70-1.43 (m, 5H, Cy-H, CyEt-H, Lac-H3), 1.41-1.12 (m, 9H, Cy-H, CyEt-H, Lac-H3, CH₂CH₃), 1.05 (m, 1H, Cy-H or CyEt-H), 0.99-0.80 (m, 4H, Cy-H, CyEt-H), 0.79-0.49 (m, 4H, Cy-H or CyEt-H, CH₂CH₃); ¹³C NMR (126 MHz, CD₃OD): δ=175.3 (PhCO), 166.6 (Lac-C1), 134.4, 131.6, 130.8, 129.7 (Ar—C, Fuc-C6, CF₂), 101.4 (Gal-C1), 99.9 (Fuc-C1), 82.7 (Gal-C3), 82.2 (CyEt-C1), 80.5 (CyEt-C2), 77.3 (Lac-C2), 76.1 (Gal-C5), 72.8 (Gal-C2), 70.3 (m, Fuc-C5), 70.0 (Fuc-C2), 69.8 (Fuc-F3), 69.4 (Fuc-C4), 67.0 (Gal-C4), 64.4 (CH₂CF₂), 62.7 (Gal-C6), 61.4 (CH₂CF₂), 45.9, 41.1, 34.9, 34.2 33.5, 32.2, 30.8, 29.4, 27.2, 26.8, 26.7, 24.8, 23.4, 10,9 (CyEt-C, Cy-C, Lac-C3, CH₂CH₃); ¹⁹F NMR (470 MHz, CD₃OD): δ=−73.6 (d, J=7.1 Hz, 3F, CF₃), −102.4-−103.77 (m, 2H, CF₂); ESI-MS: m/z: Calcd for C₃₉H₅₄F₅NNaO₁₃ [M+Na]⁺: 862.3, found: 862.3.

### Example 9

### Synthesis of Compound 53

Synthesis of compound 52: To a solution of 51 (5 mg, 0.006 mmol) in dry MeOH (0.2 mL) 1 M NaOMe (30 μL, 0.030 mmol) was added. The reaction was stirred under argon at rt for 3 d, then at 60° C. for an additional 1 h. The reaction was diluted with MeOH (1 mL) and neutralized with Amberlite IR120 resin (H⁺ form). The resin was filtered off and the filtrate was concentrated in vacuo. The crude residue was dissolved in pyridine/Ac₂O (1:1, 0.6 mL) and stirred at rt for 18 h. The reaction was diluted with toluene (10 mL) and concentrated in vacuo. The crude product was purified by flash chromatography (petroleum ether/EtOAc, gradient 0-50%) to isolate 52 (4 mg, 78%, 2 steps). ¹H NMR (500 MHz, CDCl₃): δ=5.72 (s, 1H, Fuc-H4), 5.51 (q, J=6.4 Hz, 1H, Fuc-H5), 5.46 (d, J=2.7 Hz, 1H, Gal-H4), 5.34-5.39 (m, 3 H, Fuc-H1, Fuc-H2, Fuc-H3), 4.70 (d, J=7.9 Hz, 1H, Gal-H1), 4.47-4.37 (m, 2H, Gal-H6, Lac-H2), 4.31 (dd, J=7.9, 10.2 Hz, 1H, Gal-H2), 4.19 (dd, J=7.6, 11.5 Hz, 1H, Gal-H6′), 3.90 (dd, J=6.9 Hz, 1H, Gal-H5), 3.77 (dd, J=3.3, 10.2 Hz, 1H, Gal-H3), 3.63 (m, 1H, CyEt-H1), 3.45 (t, J=9.0 Hz, 1H, CyEt-H2), 2.15, 2.12, 2.09, 2.08 (4s, 12H, 4 CH₃CO), 2.06 (m, 1H, CyEt-H), 1.98 (s, 3H, CH₃CO), 1.81-1.36 (m, 15H, CyEt-H Cy-H, Lac-H3, CH₂CH₃), 1.31-1.08 (m, 4H, Cy-H, CyEt-H, CH₂CH₃), 1.02-0.76 (m, 5H, Cy-H, CyEt-H, CH₂CH₃); ¹⁹F NMR (470 MHz, CDCl₃): δ=−72.9 (d, J=6.4 Hz, 3F, CF₃); ESI-MS: m/z: Calcd for C₃₉H₅₅F₃NaO₁₇ [M+Na]⁺: 875.3, found: 875.4.

Synthesis of compound 53: To a solution of 52 (4.0 mg, 0.0047 mmol) in DCM (0.4 mL) azetidine (0.1 mL) was added. The reaction was stirred at rt for 1 h then toluene (10 mL) was added and the mixture was concentrated in vacuo. The crude residue was lyophilized from water and purified by flash chromatography (DCM/MeOH, gradient 0-40%, MeOH contains 10% H₂O) to isolate product 53 (2.9 mg, 88%). ¹H NMR (500 MHz, D₂O): δ=5.35 (m; 1H, Fuc-H5), 5.29 (d, J=3.7 Hz, 1H, Fuc-H1), 4.47-4.35 (m, 3H, Gal-H1, Lac-H2, CH₂CF₂), 4.32 (m, 1H, NCH₂CH₂), 4.28 (d, J=2.3 Hz, 1H, Fuc-H4), 4.13-4.00 (m, 2H, NCH₂CH₂), 3.96 (dd, J=3.2, 10.5 Hz, 1H, Fuc-H3), 3.92-3.87 (m, 2H, Fuc-H2, Gal-H4), 3.78-3.66 (m, 3H, Gal-H6, CyEt-H1), 3.61-3.53 (m, 2H, Gal-H2, Gal-H5), 3.47 (t, J=9.6 Hz, 1H, CyEt-H2), 3.37 (dd, J=3.2, 9.6 Hz 1H, Gal-H3), 2.43-2.30 (m, 2H, NCH₂CH₂), 2.10 (m, 1H, CyEt-H), 1.88-1.60 (m, 9H, Cy-H, CyEt-H, Lac-H3, CH₂CH₃), 1.58-1.12 (m, 9H, Cy-H, CyEt-H, CH₂CH₃), 1.09-0.93 (m, 3H, Cy-H, CyEt-H), 0.90 (t, J=7.40 Hz, 3H, Cy-H, CH₂CH₃); ¹³C NMR (126 MHz, D₂O): δ=174.4 (Lac-C1), 101.1 (Gal-C1), 99.5 (Fuc-C1), 81.5 (Gal-C3), 80.6, 80.5 (CyEt-C1, CyEt-C2), 74.3 (2C, Lac-C2, Gal-C5), 70.0 (Gal-C2), 68.5 (m, Fur-C5), 68.0 (Fuc-C2), 67.7 (Fuc-C3), 67.3 (Fuc-C4), 67.0 (Gal-C4), 61.5 (Gal-C6), 51.2 (NCH₂CH₂), 48.8 (NCH₂CH₂), 44.7, 39.6, 33.5, 33.1 30.7, 28.3, 26.0, 25.8, 25.6, 23.1, 22.6 15.3, 9.5 (CyEt-C, Cy-C, Lac-C3, NCH₂CH₂, CH₂CH₃); ¹⁹F NMR (470 MHz, CD₃OD): δ=−72.3 (d, J=6.7 Hz, 3F, CF₃); ESI-MS: m/z: Calcd for C₃₂H₅₂F₃NNaO₁₂ [M+Na]⁺: 722.3, found: 722.4.

### Example 10

### Synthesis of Compound 59

Synthesis of compound 54: To a solution of 45 (450 mg, 0.73 mmol) in pyridine (5 mL) was added BzCl (0.17 mL, 1.45 mmol) dropwise under argon at 0° C. The mixture was stirred at rt for 4 h, then toluene (20 ml) was added and the mixture was concentrated in vacuo. The crude residue was purified by flash chromatography (petroleum ether/EtOAc, gradient 0-40%) to yield 54 (380 mg, 72%). [α]_{D}²² −8.5 (c 1.0, CHCl₃); ¹H NMR (500 MHz, CDCl₃): δ=8.09-8.03 (m, 2H, Ar—H), 7.60-7.55 (m, 3H, Ar—H), 7.43 (t, J=7.8 Hz, 2H, Ar—H), 7.40-7.28 (m, 8H, Ar—H), 6.90-6.85 (m, 2H, Ar—H), 6.73-6.67 (m, 2H, Ar—H), 5.82 (dd, J=8.0, 10.0 Hz, 1H, H-2), 5.43 (s, 1H, PhCH), 5.12 (A of AB, J=12.1 Hz, 1H, PhCH₂), 5.04 (B of AB, J=12.1 Hz, 1H, PhCH₂), 4.94 (d, J=8.0 Hz, 1H, H-1), 4.34-4.27 (m, 2H, H-4, H-6), 4.20, (dd, J=4.4, 8.7 Hz, 1H, Lac-H2), 3.96 (dd, J=1.4, 12.2 Hz, 1H, H-6′), 3.74 (dd, J=8.1, 11.4 Hz, H-3), 3.71 (s, 3H, OCH₃), 3.39 (br s, 1H, H-5), 1.62-1.54 (m, 2H, Cy-H, Lac-H3), 1.45-1.34 (m, 4H, Cy-H, Lac-H3′), 1.32-1.19 (m, 2H, Cy-H), 0.96-0.85 (m, 2H, Cy-H), 0.77 (m, 1H, Cy-H), 0.65-0.54 (m, 2H, Cy-H); ¹³C NMR (126 MHz, CDCl₃): δ=173.3 (Lac-C1), 164.9 (PhCO), 155.5, 151.5, 137.7, 135.5, 133.2, 129.9, 129.8, 128.9, 128.7, 128.6, 128.4, 128.1, 126.6, 119.4, 114.3, (Ar—C), 101.7 (C-1), 101.0 (PhCH), 78.9 (Lac-C2), 78.6 (C-3), 75.0 (C-4), 71.6 (C-2), 68.8 (C-6), 66.9 (C-5), 66.6 (PhCH₂), 55.6 (OCH₃), 40.7 (Lac-C3), 33.6, 33.3, 32.6 26.2, 25.8, 25.6 (Cy-C); ESI-MS: Calcd for C₄₃H₄₆NaO₁₀ [M+Na]⁺: 745.3 found: 745.3.

Synthesis of compound 55: To a solution of 54 (284 mg, 0.39 mmol) in MeCN/toluene (1:1, 4 ml) was added a solution of CAN (2.15 g, 3.92 mmol) in 1120 mL at −15° C. The reaction was stirred at rt for 1 h at −15° C. then brine (10 mL) was added and the resulting mixture was extracted with EtOAc (3×30 mL). The combined organic phases were washed with said. aq. NaHCO₃, dried over Na₂SO₄ and concentrated in vacua. The crude residue was purified by flash chromatography (toluene/acetone, gradient 2-20%) to isolate the intermediate (142 mg, 59%). To a solution of the intermediate (57 mg, 0.092 mmol) in dry DCM (1 mL) were added F₃CC(NPh)Cl (60 μL, 0.369 mmol) and Cs₂CO₃ (28 mg, 0.184 mmol) under argon at rt. The reaction was stirred for 18 h at rt, then the mixture was purified directly by a short flash chromatography (petroleum ether/EtOAc, gradient 0-50%) to give 55 as a mixture of α/β anomers (55 mg, 75%, 44% after 2 steps). Major anomer: ¹H NMR (500 MHz, CDCl3): δ=8.07 (d, J=7.3 Hz, 2H, Ar—H), 7.62-7.55 (m, 3H, Ar—H), 7.46 (t, J=7.8 Hz, 2H, Ar—H), 7.38-7.29 (m, 8H, Ar—H), 7.23 (t, J=7.8 Hz, 2H, Ar—H), 7.07 (m, 1H, Ar—H), 6.67 (br s, 2H, Ar—H), 5.84 (br s, 1H, H-1, H-2), 5.42 (s, 1H, PhCH), 5.14 (A of AB, J=12.1 Hz, 1H, PhCH₂), 5.04 (B of AB, J=12.1 Hz, 1H, PhCH₂), 4.33-4.14 (m, 3H, H-6, Lac-H2, H-4), 3.92 (m, 1H, H-6′), 3.75 (br s, H-3), 3.38 (br s, 1H, H-5), 1.60-1.25, (m, 8H, Cy-H, Lac-H3), 1.10-0.90 (m, 2H, Cy-H), 0.94-0.65 (m, 3H, Cy-H); ESI-MS: m/z: Calcd for C₄₄H₄₄F₃NNaO₉ [M+Na]⁺: 810.3 found: 810.2.

Synthesis of compound 56: A suspension of donor 55 (56 mg, 0.071 mmol), acceptor 47 (17.5 mg, 0.028 mmol) and activated 4 Å molecular sieves (0.3 g) in DCM (1 mL) was stirred at rt for 1 h under argon. The suspension was cooled to −50° C. and a solution of TMSOTf (1.57 μL, 8.5 μmol) in DCM (50 μL) was added. The reaction was allowed to warns up to −20° C. (2 h) and then was quenched with Et₃N (0.1 mL). The mixture was purified directly by flash chromatography (toluene/EtOAc, gradient 0-30%) to isolate product 56 (28.5 mg, 84%). [α]_{D}²² −65.0 (c 0.50, CHCl₃); ¹H NMR (500 MHz, CDCl₃): δ=8.06 (m, 2H, Ar—H), 7.61 (d, J=7.3 Hz, 2H, Ar—H), 7.55 (t, 7.4 Hz, 1H, Ar—H), 7.43 (t, J=7.7 Hz, 2H, Ar—H), 7.37-7.12 (m, 23H, Ar—H), 5.46-5.39 (m, 2H, PhCH, Gal-H2), 5.29 (q, J=6.3 Hz, 1H, Fuc-H5), 5.07 (A of AB, J=12.2 Hz, 1H, PhCH₂), 5.05 (d, J=3.0 Hz, 1H, Fuc-H1), 4.99 (B of AB, J=12.2 Hz, 1H, PhCH₂), 4.80 (A′ of A′B′, J=11.7 Hz, 1H, PhCH₂), 4.71-4.60 (m, 4H, Gal-H1, B′ of PhCH₂, A″B″ of PhCH₂), 4.33 (d, J=12.1 Hz, 1H, Gal-H6), 4.24 (d, J=3.3 Hz, 1H, Gal-H4), 4.17 (A′″ of A′″B′″, J=10.7 Hz, 1H, PhCH₂), 4.12 (m, 1H, Lac-H2), 4.02-3.93 (m, 3H, Gal-H6′, Fuc-H2, Fuc-H3), 3.86 (s, 1H, Fuc-H4), 3.80-3.74 (m, 2H, Gal-H3, B′″ of PhCH₂), 3.52 (m, 1H, CyEt-H1), 3.32 (t, J=9.6 Hz, 1H, CyEt-H2), 3.28 (br s, 1H, Gal-H5), 1.91-1.80 (m, 2H, CyEt-H or Cy-H), 1.66-1.80 (m, 2H, Lac-H3, CH₂CH₃), 1.54-1.31 (m, 9H, Lac-H3′, Cy-H, CyEt-H), 1.20-0.78 (m, 7H, Cy-H, CyEt-H, CH₂CH₃), 0.77 (t, J=7.3 Hz, 1H, CH₂CH₃), 0.70-0.56 (m, 2H, Cy-H or CyEt-H); ¹³C NMR (126 MHz, CDCl₃): δ=173.4 (Lac-C1), 164.5 (PhCO), 139.3, 139.1, 138.5, 137.9, 135.7, 133.1, 130.3, 129.9, 128.8, 128.7, 128.6, 128.6, 128.5, 128.4, 128.3, 128.0, 127.9, 127.7, 127.6, 127.4, 127.1, 126.3 (Ar—C, Fuc-C6) 101.0 (Gal-C1), 100.1 (PhCH), 97.9 (Fuc-C1), 82.6 (CyEt-C1), 79.2 (Gal-C3), 79.0 (CyEt-C2), 79.0 (CyEt-C2), 78.7 (Lac-C2), 78.3 (Fuc-C3), 75.4 (Fuc-C2), 75.1 (Gal-C4), 74.9, 74.7 (2 PhCH₂), 74.3 (Fuc-C4), 72.5 (Gal-C2), 71.7 (PhCH₂), 69.2 (Gal-C6), 68.6 (m, Fuc-C5), 66.0 (Gal-C5), 66.6 (PhCH₂), 45.6, 40.8, 33.7, 33.4, 32.8, 31.6, 28.5, 26.4, 25.9, 25.7, 23.5, 23.3, 21.2, 10.6 (CyEt-C, Cy-C, Lac-C3, CH₂CH₃); ¹⁹F NMR (470 MHz, CDCl₃): δ=−71.6 (d, J=6.8 Hz, 3F, CF₃); ESI-MS: m/z: Calcd for C₇₁H₇₉F₃NaO₁₄ [M+Na]⁺: 1235.5 found: 1235.7.

Synthesis of compound 57: To a solution of 56 (25 mg, 0.021 mmol) in THF (0.5 mL) 1 M NaOH (0.2 mL) was added. The reaction was stirred at 60° C. for 6 h, then MeOH (2 mL) was added and the reaction was neutralized with Amberlite IR120 resin (H⁺ form). The resin was filtered off and the filtrate was concentrated in vacuo. The crude residue was dissolved in pyridine/Ac₂O (1:1, 1 mL) and stirred at rt for 3 h. The reaction was co-evaporated with toluene (2×10 mL). The crude product was dissolved in DCM (2 mL) and azetidine (0.2 mL) was added. The reaction was stirred at rt for 18 h then purified directly by flash chromatography (petroleum ether/acetone, gradient 3-60%) to isolate 57 (9.7 mg, 44%, 3 steps). [α]_{D}²² −55.8 (c 0.43, CHCl₃); ¹H NMR (500 MHz, CDCl₃): δ=7.60(d, J=7.0 Hz, 2H, Ar—H), 7.35-7.12 (m, 18H, Ar—H), 5.61 (s, 1H, PhCH), 5.32 (q, J=6.6 Hz, 1H, Fuc-H5), 5.08 (d, J=1.8 Hz, 1H, Fuc-H1), 4.81 (A of AB, J=11.7 Hz, 1H, PhCH₂), 4.64 (B of AB, J=11.7 Hz, 1H, PhCH₂), 4.55 (A′ of A′B′, J=11.3 Hz, 1H, PhCH₂), 4.46-4.40 (m, 2H, NCH₂CH₂, B′ of PhCH₂), 4.36-4.17 (m, 6H, Gal-H6, Gal-H1, NCH₂CH_{2,} Gal-H4, Lac-H2, A″ of PhCH₂), 4.08 (dd, J=1.4, 12.2 Hz, 1H, Gal-H6′), 4.03-3.97 (m, 2H, Fuc-H2, Fuc-H3), 3.95-3.86 (m, 4H, B″ of PhCH₂, Gal-H2, Fuc-H4, NCH₂CH₂), 3.73-3.59 (m, 2H, NCH₂CH₂, CyEt-H1), 3.46 (dd, J=3.5, 9.7 Hz, 1H, Gal-H3), 3.39 (t, J=9.7 Hz, 1H CyEt-H2), 3.35 (br s, 1H, Gal-H5), 2.32 (d, J=1.6 Hz, 1H, OH), 2.17-2.04 (m, 2H, CyEt-H, NCH₂CH₂), 1.95 (m, 1H, CH₂CH₃), 1.85-1.57 (m, 9H, Lac-H3, Cy-H, CyEt-H), 1.55-1.08 (m, 9H, Cy-H, NCH₂CH₂, CyEt-H, CH₂CH₃), 1.04-0.87 (m, 3H, Cy-H, CyEt-H), 0.84 (t, J=7.4 Hz, 3H, CH₂CH₃); ¹³-C NMR (126 MHz, CDCl₃): δ=172.8 (Lac-C1), 139.1, 138.9, 138.4, 138.3, 129.2, 129.0, 128.8, 128.4, 128.3, 128.2, 128.1, 127.8, 127.8, 127.6, 127.3, 127.3, 126.3 (Ar−C, Fuc-C6), 102.2 (Gal-C1), 100,5 (PhCH), 98.1 (Fuc-C1), 81.2 (CyEt-C1), 80.6 (Gal-C3), 79.6 (CyEt-C2), 78.4 (Lac-C2), 78.4 (Fuc-C3,), 75.1 (Fuc-C2), 74,9 (PhCH₂), 74.7 (2C, Gal-C4, PhCH₂), 74.2 (Fuc-C4), 71.9 (PhCH₂), 70.1 (Gal-C2), 69.5 (Gal-C6), 68.8 (m, Fuc-C5), 67.0 (Gal-C5), 51.3, 48.6 (NCH₂CH₂), 45.7 (NCH₂CH₂), 40.2, 34.1, 34.0, 32.7, 31.6, 28.8, 26.7, 26.4, 26.3, 23.5, 23.4 (CyEt-C, Cy-C, Lac-C3, CH₂CH₃), 16.2 (CH₂CH₃); ¹⁹F NMR (470 MHz, CDCl₃): δ=−71,7 (d, J=6.8 Hz, 3F, CF₃); ESI-MS: m/z: Calcd for C₆₀H₇₄F₃NNaO₁₂ [M+Na]⁺: 1080.5, found: 1080.6.

Synthesis of compound 58: A solution of 57 (20 mg, 0.019 mmol) in pyridine/Ac₂O (1:1, 1 mL) was stirred at rt for 18 h. The reaction was co-evaporated with toluene (2×10 mL) and the crude product was purified by flash chromatography (petroleum ether/acetone, gradient 3-50%) to isolate 58 (17.8 mg, 85%). [α]_{D}²² −33.8 (c 0.50, CHCl₃); ¹H NMR (500 MHz, CDCl₃): δ=7.65 (d, J=7.3 Hz, 2H, Ar—H), 7.37 (t, J=7.4 Hz, 2H, Ar—H), 7.34-7.13 (m, 16H, Ar—H), 5.62 (s, 1H, PhCH), 5.32 (q, J=6.6 Hz, 1H, Fuc-H5), 5.23 (m, 1H, Gal-H2), 5.09 (d, J=2.5 Hz, 1H, Fuc-H1), 4.82 (A of AB, J=11.8 Hz, 1H, PhCH₂), 4.70-4.60 (m, 2H, NCH₂CH₂, B of PhCH₂), 4.57 (A′ of A′B′, J=11.3 Hz, 1H, PhCH₂), 4.47 (d, J=8.0 Hz, 1H, Gal-H1), 4.46 (B′ of A′B′, J=11.3 Hz, 1H, PhCH₂), 4.35 (d, J=11.9 Hz, 1H, Gal-H6), 4.32 (d, J=3.4 Hz, PH, Gal-H4), 4.26 (A″ of A″B″, J=10.8 Hz, 1H, PhCH₂), 4.14 (td, J=6.0, 9.5 Hz, 1H, NCH₂CH₂), 4.08 (d, J=11.9 Hz, 1H, Gal-H6′), 4.04-3.96 (m, 4H, Fuc-H2, Fuc-H3, Fuc-H4, B″ of PhCH₂,), 3.87 (dd, J=3.9, 9.7 Hz, 1H, Lac-H2), 3.77 (m, 1H, NCH₂CH₂), 3.60-3.51 (m, 2H, Gal-H3, CyEt-H1), 3.47 (m, 1H, NCH₂CH₂), 3.42 (t, J=9.6 Hz, 1H CyEt-H2), 3.35 (br s, 1H, Gal-H5), 2.08 (s, 3H, COCH₃), 2.07-1.95 (m, 2H, Cy-H or CyEt-H, NCH₂CH₂), 1.90 (m, 1H, CH₂CH₃), 1.75-1.53 (m, 9H, Cy-H, CyEt-H, NCH₂CH₂, Lac-H3), 1.53-1.36 (m, 3H, Lac-H3′, Cy-H, CyEt-H), 1.34-1.10 (m, 6H, Cy-H, CyEt-H, CH₂CH₃), 1.00-0.85 (m, 3H, Cy-H, CyEt-H), 0.83 (t, J=7.3 Hz, 3H, CH₂CH₃); ¹³C NMR (126 MHz, CDCl₃): δ=171.9 (Lac-C1), 168.5 (PhCO), 138.9, 138.8, 138.3, 137.8, 129.1, 129.0, 128.6, 128.2, 128.2, 128.2, 127.9, 127.7, 127.7, 127.5, 127.2, 127.1 126.3 (Ar—C, Fuc-C6), 101.0 (Gal-C1), 100.8 (PhCH), 97.7 (Fuc-C1), 82.7 (CyEt-C1), 81.0 (Gal-C3), 80.3 (Lac-C2), 78.6 (CyEt-C2), 78.3 (Fuc-C3), 75.0 (Fuc-C2), 74.7, 74.6 (2 PhCH₂), 74.0 (Fuc-C4), 73.6 (Gal-C4), 71.6 (PhCH₂), 69.8 (Gal-C2), 69.3 (Gal-C6), 68.5 (m, Fuc-C5), 66.5 (Gal-C5), 52.0, 48.5 (NCH₂CH₂), 45.5 (NCH₂CH₂), 40.0, 34.0, 33.6, 32.5, 31.5, 28.6, 26.4, 26.3, 26.1, 23.5, 23.2, 21.0 (CyEt-C, Cy-C, Lac-C3, CH₂CH₃), 16.1 (CH₂CH₃); ¹⁹F NMR (470 MHz, CDCl3): δ=−71.8 (d, J=6.8 Hz, 3F, CF₃); ESI-MS: m/z: Calcd for C₆₂H₇₆F₃NNaO₁₃ [M+Na]⁺: 1122.5, found: 1122.6.

Synthesis of compound 59: A suspension of 58 (17 mg, 0.0154 mmol) and Pd(OH)₂/C (20 mg, 10% Pd) in THF (4 mL) was hydrogenated (1 bar H₂) for 2 h at rt. The reaction mixture was filtered through a double layer of filter paper and concentrated in vacuo. The crude residue was purified by flash chromatography (DCM/MeOH, gradient 5-25%) to isolate product 59 (9.7 mg, 85%). [α]_{D}²² −71.1 (c 0.25, MeOH); ¹H NMR (500 MHz, CD₃OD): δ=5.20 (q, J=6.9 Hz, 1H, Fuc-H5), 5.17 (d, J=3.8 Hz, 1H, Fuc-H1), 5.11 (dd J=8.3, 9.5 Hz, 1H, Gal-H2), 4.47 (d, J=8.1 Hz, 1H, Gal-H1), 4.39-4.29 (m, 2H, NCH₂CH₂), 4.18 (d, J=2.1 Hz, 1H, Fuc-H4), 4.11-3.99 (m, 3H, NCH₂CH₂, Lac-H2), 3.96 (dd, J=3.2, 10.3 Hz, 1H, Fuc-H3), 3.86-3.81 (m, 2H, Fuc-H2, Gal-H4), 3.81-3.71 (m, 2H, Gal-H6), 3.59 (m, 1H, CyEt-H1), 3.52-3.46 (m, 2H, Gal-H5, Gal-H3), 3.42 (t, J=8.9 Hz, 1H, CyEt-H2), 2.44-2.31 (m, 2H, NCH₂CH₂), 2.09 (s, 3H, COCH₃), 2.02 (m, 1H, CyEt-H), 1.95-1.81 (m, 2H, CyEt-H or Cy-H, CH₂CH₃), 1.80-1.16 (m, 16H, Cy-H, CyEt-H, CH₂CH_{3,} Lac-H3), 1.12-0.94 (m, 3H, Cy-H, CyEt-H), 0.92 (t, J=7.4 Hz, 3H, CH₂CH₃); ¹³C NMR (126 MHz, CD₃OD): δ=175.5, 171.1 (COCH₃, Lac-C1), 101.8 (Gal-C1), 99.8 (Fuc-C1), 83.0 (Gal-C3), 82.4 (CyEt-C1), 80.2 (CyEt-C2), 76.0 (Gal-C5), 75.8 (Lac-C2), 72.7 (Gal-C2), 70.2 (m, Fuc-C5), 69.9 (Fuc-C2), 69.8 (Fuc-C3), 69.3 (Fuc-C4), 67.5 (Gal-C4), 62.6 (Gal-C6), 52.3, 49.8 (NCH₂CH₂), 45.9, 41.8, 35.2, 34.7 33.4, 32.2, 30.8, 29.5, 27.5, 27.4, 27.2, 24.8, 23.5, 21.5, 16.8 (CyEt-C, Cy-C, Lac-C3, NCH₂CH₂, COCH₃, CH₂CH₃), 10.9 (CH₂CH₃); ¹⁹F NMR (470 MHz, CD₃OD): δ=−73.8 (d, J=7.1 Hz, 3F, CF₃); EST-MS: m/z: Calcd for C₃₄H₅₄F₃NNaO₁₃ [M+Na]⁺: 764.3, found: 764.3.

### Example 11

### Synthesis of Compound 69

Synthesis of compound 61: A stirred solution of tri-O-acetyl-D-galactal (60, 2.92 g, 10.7 mmol) in DCM (20 mL) was cooled in an ice bath and freshly prepared chlorine (concentrated HO was added to solid KMnO₄ at rt to provide Cl₂) was bubbled in, until a color caused by excess chlorine was present. After the solution was stirred for another 30 min, air was bubbled into the solution to remove the excess chlorine, and the solvent was evaporated under reduced pressure to provide the intermediate dichloride, which was used in the next step without further purification. To a solution of the dichloride (10.7 mmol) in dry MeOH (13 mL) was added silver carbonate at rt and the suspension was stirred at rt for another 2.5 h. The silver salt was removed by filtration through celite and the filter cake was washed thoroughly with MeOH, The filtrate was evaporated in vacuum and the residue was purified by flash chromatography (petroleum ether/EtOAc, 4:1 to 3:1) to provide 61 (2.93 g, 80%, 2 steps) as white solid. ¹H NMR (500 MHz, CDCl₃): δ=5.36 (d, J=3.1 Hz, 1H , H-4), 5.02 (dd, J=3.3, 11.0 Hz, 1H, H-3), 4.42 (d, J=8.3 Hz, 1H, H-1), 4.20 (m, 1H, H-6a), 4.12 (m, 1H, H-6b), 3.96 (t, J=6.7 Hz, 1H H-5), 3.91 (dd, J=8.4, 10.9 Hz, 1H, H-2), 3.60 (s, 3H, OMe), 2.13, 2.06, 2.05 (3s, 9H, 3 COCH₃); ESI-MS: m/z: Calcd for C₁₃H₁₉ClNaO₈ [M+Na]⁺: 361.1, found: 361.0.

Synthesis of compound 62: To a solution of 61 (1.83 g, 5.40 mmol) in acetic anhydride (15.0 mL) was added conc. H₂SO₄ (0.433 mL) at rt. The reaction mixture was stirred at rt for 18 h, then diluted with EtOAc and quenched carefully with satd. aq. NaHCO₃ at 0° C. The aqueous layer was extracted with EtOAc (2×50 mL), and the combined organic layers were washed with brine and dried over Na₂SO₄. The solvent was removed in vacuum and the residue was purified by chromatography on silica gel (petroleum ether/EtOAc, 4:1 to 3:1) to provide 62 (1.62 g, 81%) as white solid. ³H NMR (500 MHz, CDCl₃): δ=6.37 (d, J=3.4 Hz, 1H, H-1α), 5.75 (d, J=8.8 Hz, 0.38H, H-1β), 5.46 (d, J=1.8 Hz, 1H, H-4α), 5.39 (d, J=2.8 Hz, 0.36H, H-4β), 5.32 (dd, J=3.2, 11.2 Hz, 1H, H-3α), 5.09 (dd, J=3.3, 10.9 Hz, 0.37H, H-3β), 4.35 (t, J=7.1 Hz, 1H, H-5α), 4.32 (dd, J=3.5, 11.3 Hz, 1H, Hαa), 4.23-3.99 (m, 3.5H, H-6aα, H-6bα, H-6aβ, H-6Hβ, H-5β, H-2β), 2.19, 2.16, 2.06, 2.04 (4s, 16H, 4 COCH₃); ESI-MS: m/z: Calcd for C₁₄H₁₉ClNaO₉ [M+Na]⁺: 389.1, found: 389.0.

Synthesis of compound 63: To a solution of 62 (1.19 g, 3.24 mmol) in dry DMF (5.0 mL) was added ammonium acetate (748 mg, 9.72 mmol) at rt. The reaction mixture was stirred at rt for 18 h, then diluted with EtOAc, washed with water and brine, and dried over Na₂SO₄. The solvents were removed in vacuum and the residue was purified by chromatography on silica gel (petroleum ether/EtOAc, 7:3 to 6:4) to give 63 (0.67 g, 64%) as colorless oil, which was directly used in the next step.

Synthesis of compound 64: To a mixture of 63 (653 mg, 2.00 mmol) and trichioroacetonitrile (2.0 mL) in dry DCM (10 mL) was added DBU (89.6 μL, 0.6 mmol) at rt under argon. The reaction mixture was stirred at rt for 1.5 h, then the solvent was removed in vacuum and the residue was purified by chromatography on silica gel (petroleum ether/EtOAc, 4:1 to 3:1) to yield 64 (752 mg, 81%) as white foam. [α]_{D}²⁰ +131.1 (c 1.46, DCM); NMR (500 MHz, CDCl₃): δ=8.77 (s, 1H, NH), 6.53 (d, J=3.2 Hz, 1H, H-1), 5.48 (d, J=1.8 Hz, 1H, H-4), 5.34 (dd, j=3.1, 11.2 Hz, 1H, H-3), 4.45 (t, J=6.6 Hz, 1H, H-5), 4.36 (dd, J=3.3, 11.2 Hz, 1H, H-3), 4.11 (dd, J=6.6, 11.3 Hz, 1H, H-6a), 4.02 (dd, J=6.7, 11.3 Hz, 1H, H-6b), 2.12, 2.02, 1.97 (3s, 9H, COCH₃); ¹³C NMR (126 MHz, CDCl₃): δ=170.1, 169.7, 169.5 (3 CO), 160.4 (CN), 94.8 (C-1), 90.6 (C13C), 69.5 (C-3), 69.1 (C-5), 67.4 (C-4), 61.0 (C-6), 53.5 (C-2), 20.46, 20.41, 20.39 (3 COCH₃); ESI-MS: m/z: Calcd for C₁₄H₁₇NNaO₈ [M+Na]⁺: 490.0; found: 490.0.

Synthesis of compound 65: To a mixture of 64 (218 mg, 0.464 mmol), 47 (93.0 mg, 0.15 mmol) and 4 Å molecular sieves in dry DCM/CH₃CN (1:1, 8 mL) was added TMSOTf (25.1 μL, 0.139 mmol) at −18° C. under argon. The mixture was stirred at −18° C. for 1 h, then at rt for another 4 h. The reaction mixture was diluted with DCM and quenched with Et₃N, filtered through celite, and the celite was washed with DCM. The filtrate was concentrated under reduced pressure and the residue was purified by silica gel chromatography (petroleum ether/EtOAc, 6:1 to 3:1) to yield an inseparable mixture of α/β-isomers (104 mg) as white foam. To a suspension of the intermediate (104 mg) in dry MeOH (4.0 mL) was added MeONa/MeOH (0.5 N, 0.35 mL) at rt. The reaction mixture was stirred at rt for 1 h, then neutralized with HOAc. The solvents were removed in vacuum and the residue was purified by chromatography on silica gel (DCM/MeOH, 24:1 to 19:1) to give 65 (41 mg, 34%, 2 steps) as white foam. [α]_{D}²⁰ −87.5 (c 1.0, DCM); ¹H NMR (500 MHz, CDCl₃): δ=7.50-7.15 (m, 15H, Ar—H), 5.25 (q, J=6.4 Hz, 1H, Fuc-H5), 5.21 (d, J=3.5 Hz, 1H, Fuc-H1), 4.89 (d, 10.8 Hz, 1H, CH₂Ph), 4.85 (d, J=11.5 Hz, 1H, CH₂Ph), 4.81 (s, 2H, CH₂Ph), 4.70 (d, J=11.5 Hz, 1H, CH₂Ph), 4.65 (d, J=10.8 Hz, 1H, CH₂Ph), 4.41 (d, J=8.4 Hz, 1H, Gal-H1), 4.30 (s, 1H, Fuc-H4), 4.17 (dd, J=3.6, 10.3 Hz, 1H, Fuc-H2), 4.07 (dd, J=2.3, 10.3 Hz, 1H, Fuc-H3), 4.01 (s, 1H, Gal-H4), 3.90 (dd, J=4.9, 11.7 Hz, 1H, Gal-H6a), 3.85-3.74 (m, 2H, Gal-H2, Gal-H6b), 3.65-3.56 (m, 2H, Gal-H3, Cy-H1), 3.52-3.41 (m, 2H, Gal-H5, Cy-H2), 2.06 (m, 1H), 1.91 (m, 1H), 1.72 (m, 2H), 1.50 (m, 1H), 1.41 (m, 1H), 1.27 (m, 1H), 1.16 (m, 1H), 0.94 (m, 1H), 0.83 (t, 7.3 Hz, 3H, CH₃); ¹³C NMR (126 MHz, CDCl₃): δ=138.8, 138.3, 138.0, 128.5, 128.3, 128.3, 128.1, 128.1, 127.8, 127.5, 127.3 (Ar—C, Fuc-C6), 100.9 (Gal-C1), 98.0 (Fuc-C1), 81.1 (Cy-C1), 80.2 (Cy-C2), 78.6 (Fuc-C3), 75.6 (Fuc-C2), 74.9, 74.8 (2 CH₂Ph), 74.7 (Gal-C3), 74.0 (Fuc-C4), 73.7 (Gal-C5), 72.3 (CH₂Ph), 70.0 (Gal-C4), 68.7 (Fuc-C5), 63.2 (Gal-C6), 61.2 (Gal-C2), 45.11, 31.0, 28.5, 23.4, 22.8, 10.5 (CH₃); ESI-MS: m/z: Calcd for C₄₁H₅₀ClF₃NaO₁₀ [M+Na]⁺: 817.3, found: 817.3.

Synthesis of compound 67: A suspension of 65 (39.0 mg, 0.049 mmol) and n-Bu₂SnO (14 mg, 0.056 mmol) in dry MeOH (3 mL) was refluxed for 3 h. The solvent was removed under reduced pressure and the resulting foam was dried in vacuo for 4 h, CsF (15.0 mg, 0.098 mmol) was dried in vacuo at 100° C. for 30 min and flushed with argon. Then 66 (D. Hesek et al., J. Org. Chem., 71, 5848-5854 (2006)) (34.7 mg, 0.0989 mmol), CsF and DME (1.0 mL) were added to the tin acetal at rt. The reaction mixture was stirred at rt for 18 h. The solvent was removed under reduced pressure and the residue was purified by silica gel chromatography (petroleum ether/EtOAc, 4:1 to 7:3) to afford 67 (12.4 mg, 25%, 2 steps) as white foam. [α]_{D}²⁰ −87.3 (c 1.24, DCM); ¹H NMR (500 MHz, CDCl₃): δ=7.46-7.15 (m, 20H, Ar—H), 5.32 (m, 1H, Fuc-H5), 5.20 (s, 2H, CH₂Ph), 5.18 (d, J=3.5 Hz, 1H, Fuc-H1), 4.88 (d, J=11.5 Hz, 1H, CH₂Ph), 4.83 (d, J=10.8 Hz, 1H, CH₂Ph), 4.79 (d, J=11.5 Hz, 1H, CH₂Ph), 4.70 (d, J=11.5 Hz, 1H, CH₂Ph), 4.65 (d, J=10.8 Hz, 1H, CH₂Ph), 4.35 (m, 2H, Fuc-H4, Gal-H1), 4.17-4.08 (m, 3H, Fuc-H2, Lac-H2, Fuc-H3), 3.89 (t, J=9.2 Hz, 1H, Gal-H2), 3.85 (dd, J=4.7, 11.7 Hz, 1H, Gal-H6a), 3.74 (m, 2H, Gal-H4, Gal-H6b), 3.63-3.54 (m, 2H, 6-OH, Cy-H1), 3.44 (t, J=9.4 Hz, 1H, Cy-H2), 3.33 (s, 1H, Gal-H5), 3.25 (dd, 2.8, 10.0 Hz, 1H, Gal-H3), 2.05 (m, 2H), 1.94 (m, 1H), 1.86-1.64 (m, 4H), 1.58-1.37 (m, 3H), 1.28-1.12 (m, 2H), 0.99-0.85 (m, 7H), 0.82 (t, J=7.3 Hz, 3H, CH₃); ¹³C NMR (126 MHz, CDCl₃): δ=174.8 (CO), 138.9, 138.5, 138.2, 135.1, 128.8, 128.8, 128.6, 128.5, 128.3, 128.3, 128.1, 128.1, 127.7, 127.4, 127.4 (Ar—C, Fuc-C6), 101.5 (Gal-C1), 98.2 (Fuc-C1), 84,6 (Gal-C3), 81.3 (Cy-C1), 80.2 (Cy-C2), 78.7 (Lac-C2), 78.5 (Fuc-C3), 75.5 (Fuc-C2), 74.9, 74.8 (2 CH₂Ph), 74.0 (Fuc-C4), 73.2 (Gal-C5), 72.2 (CH₂Ph), 68.8 (2C, Fuc-C5, Gal-C4), 67.4 (CH₂Ph), 63.4 (Gal-C6), 59.0 (Gal-C2), 45.2, 42.6, 31.1, 29.3, 28.5, 23.9, 23.4, 23.3, 21.5, 10.5 (CH₂CH₃).

Synthesis of compound 68: Compound 67 (12.4 mg, 0.0124 mmol) was dissolved in azetidine (0.2 mL) and stirred at 35° C. for 18 h. The excess of azetidine was removed by flushing with air and the residue was purified by silica gel chromatography (DCM/MeOH, 40:1 to 24:1) to afford 68 (11.6 mg, 99%) as white solid. [α]_{D}²⁰ −209 (c 0.58, DCM); ¹H NMR (500 MHz, CDCl₃): δ=7.47-7.18 (m, 15H, Ar—H), 5.32 (m, 1H, Fuc-H5), 5.18 (d, J=3.1 Hz, 1H, Fuc-H1), 4.87-4.77 (m, 4H, CH₂Ph), 4.70 (d, J=11.6 Hz, 1H, CH₂Ph), 4.64 (d, J=10.8 Hz, 1H, CH₂Ph), 4.41-4.33 (m, 2H, Fuc-H2, Gal-H1), 4.26-4.01 (m, 6H, NCH₂CH₂, Fuc-H2, Fuc-H3), 3.99-3.88 (m, 3H, Lac-H2, Gal-H2, Gal-H4), 3.79 (m, 2H, Gal-H4, Gal-H6b), 3.60 (m, 1H, Cy-H1), 3.47-3.40 (m, 2H, Cy-H2, Gal-H5), 3.31 (s, 1H, Gal-40H), 3.18 (dd, J=2.9, 9.9 Hz, 1H, Gal-H3), 2.37 (m, 2H, NCH₂CH₂), 2.19-2.02 (m, 2H), 2.00-1.66 (m, 4H), 1.52-1.37 (m, 2H), 1.32-1.09 (m, 4H), 0.96 (d, J=6.7 Hz, 3H, CHCH₃CH₃), 0.93 (d, J=6.5 Hz, 3H, CHCH₃CH₃), 0.94 (m, 1H), 0.82 (t, J=7.3 Hz, 3H, CH₂CH₃); ¹³C NMR (126 MHz, CDCl₃): δ=173.5 (CO), 138.9, 138.5, 138.2, 128.5, 128.5, 128.2, 128.1, 128.0, 127.7, 127.6, 127.4, 127.3, 127.3, 127.0 (Ar—C, Fuc-C6), 101.6 (Gal-C1), 98.1 (Fuc-C1), 84.5 (Gal-C3), 81.1 (Cy-C1), 80.0 (Cy-C2), 78.6 (Fuc-C3), 75.4 (Fuc-C2), 75.4 (Lac-C2), 74.9, 74.7 (2 CH₂Ph), 73.9 (Fuc-C4), 73.4 (Gal-C5), 72.1 (CH₂Ph), 68.6 (2C, Fuc-C5, Gal-C4), 63.5 (Gal-C6), 59.2 (Gal-C2), 50.5 (NCH₂CH₂), 48.6 (NCH₂CH₂), 45.2, 42.3, 31.1, 28.5, 23.7, 23.6, 23.4, 22.9, 21.1, 15.9, 10.5 (CH₂CH₃); ESI-MS: m/z: Calcd for C₅₀H₆₅ClF₃NNaO₁₁ [M+Na]⁺: 970.4, found: 970.4.

Synthesis of compound 69: A suspension of 68 (11.6 mg, 0.0122 nmol) and Pd(OH)₂/C (3.2 mg, 10% Pd) in dioxane (2.5 mL) was hydrogenated (1 bar H₂) at rt for 18 h. The reaction mixture was filtered through celite and the solvent was removed under reduced pressure. The residue was purified by silica gel chromatography (DCM/MeOH+10% H₂O, 6:1 to 4:1). The product 69 was dissolved in MeCN/H₂O, filtered through a 0.45 μL microfilter and the filtrate was lyophilized to provide 4 (6.2 mg, 75%) as white fluffy solid, ¹H NMR (500 MHz, CD₃OD): δ=5.25 (d, J=6.9 Hz, 1H, Fuc-H5), 5.16 (d, J=3.6 Hz, 1H, Fuc-H1), 4.49 (d, J=8.5 Hz, 1H, Gal-H1), 4.40 (m, 1H NCH₂CH₂), 4.30 (m, 1H, NCH₂CH₂), 4.22 (dd, J=2.8, 10.2 Hz, 1H), 4.17 (d, J=1.4 Hz, 1H), 4.12-3.98 (m, 2H, NCH₂CH₂), 3.92 (dd, J=3.1, 10.3 Hz, 1H), 3.81 (m, 4H), 3.71 (dd, J=5.1, 11.4 Hz, 1H), 3.65 (m, 1H), 3.52 (t, J=5.7 Hz, 1H), 3.46-3.39 (m, 2H), 2.38 (m, 2H, NCH₂CH₂), 2.12 (m, 2H), 1.96 (m, 1H), 1.74 (m, 3H), 1.53 (m, 1H), 1.43-1.21 (m, 4H), 1.05 (m, 1H), 1.02 (m, 6H, CHCH₃CH₃), 0.93 (t, J=7.4 Hz, 3H, CH₂CH₃); ESI-MS: m/z: Calcd for C₂₉H₄₇ClF₃NNaO₁₁ [M+Na]⁺: 700.3; found: 700.2.

### Example 12

### Synthesis of Compound 75

Synthesis of compound 75: To a stirred solution of compound 2 (70 g, 272.6 mmol) in pyridine (600 mL) was added 4-dimethylaminopyridine (33.3 g, 272.6 mmol) and triisopropylsilylchloride (52.6 g, 272.6 mmol) . The reaction mixture was stirred at RT for 12 h. The reaction mixture was concentrated under reduced pressure. The resultant residue was diluted with water, extracted with ethyl acetate, dried over anhydrous sodium sulfate, filtered and concentrated under reduced pressure to obtain crude compound 75 (110 g) which was used in the next step without further purification.

Synthesis of compound 76: NaH (ca. 60% in oil, 32.0 g, 800 mmol) was added portion-wise, under argon, to a solution of compound 75 (110 g, 266.5 mmol) in DMF (500 mL) at 0° C. Once hydrogen formation had ceased (about 30 min at rt), TBAI (1.1 g) was added, followed by dropwise addition of BnBr at 0° C. (95 mL, 800 mmol). The reaction mixture was warmed to room temperature and stirred overnight. MeOH was then added carefully (2 mL) and the resulting solution concentrated to dryness in vacuo. The residue was dissolved in CH₂Cl₂, washed with. H₂O and brine, dried over anhydrous sodium sulfate and filtered. After evaporation of the solvents, the residue was subjected to a quick silica gel chromatography to remove excess benzyl bromide and polar by-products to provide 62 g of partially pure compound 76.

Synthesis of compound 77: 2,6-Lutidine (168 mL, 1456 mmol) and then NBS (97.2 g, 546 mmol) was added in small portions to a stirred solution of impure compound 76 (62 g, 91 mmol) in acetone (250 mL) and H₂O (40 mL) at 0° C. After stirring for 1 h at 0° C., the reaction was carefully quenched with saturated aqueous solutions of NaHCO₃ (250 mL) and Na₂S₂O₃ (250 mL). Acetone was evaporated and the aqueous phase extracted with EtOAc. The organic phase was dried, filtered and the solvent evaporated to dryness in vacuo to give 80 g of crude compound 77. A 20 g portion of this material was purified by flash chromatography (EtOAc/cyclohexane, 1:9) to give aldehyde 77 (10.8 g) as a syrup.

Synthesis of compound 78: To a solution of compound 77 (10.8 g, 18.7 mmol) and CF₃TMS (5.6 mL, 37.5 mmol) in THF (100 mL) was added TBAF (1 M solution in THE, 1 mL, 0.94 mmol). The reaction mixture was allowed to stir at rt for 6 h and additional TBAF (1 M solution in THF, 56 mL, 56 mmol) was added. The reaction mixture was stirred overnight at rt and concentrated under reduced pressure. The resultant residue was diluted with ethyl acetate, washed (half sat. aq. NaCl), dried (Na2SO4), and concentrated. Purification by automated flash chromatography provided the desired diol (4.1 g, 45% yield, 85% purity).

Alternative Synthesis of compound 10: Trichloroisocyanuric acid (0.7 g, 2.9 mmol) was added to a solution of the compound 78 (4.0 g, 8.2 mmol) in CH₂Cl₃ (20 mL), and the solution was stirred and maintained at 0° C., followed by addition of TEMPO (0.015 g, 0.1 mmol). After the addition, the mixture was warmed to room temperature and stirred for 5 min and then filtered through Celite. The mixture was transferred to a separatory funnel and was washed with 15 mL of a saturated solution of Na₂CO₃, followed by 1 N HCl, then brine. The organic phase was dried (Na₂SO₄), and the solvent was evaporated. Purification of resultant residue by automated flash chromatography provided the compound 10 (1.7 g, 42% yield,).

Synthesis of compound 71: Compound 10 (800 mg, 1.64 mmol; 1.0 equiv.) was dissolved in anhydrous dichloromethane (15 mL). To this solution 2,2,2-trifluoro-N-phenyl-ethanimidoyl chloride (551 mg, 2.46 mmol; 1.5 equiv.) and sodium hydride (60% suspension in oil, 98 mg, 2,46 mmol; 1.5 equiv.) were added at 0° C. After three hours at 0° C. the mixture was concentrated under vacuum and separated on a silica gel column eluted with hexanes; ethyl acetate (with a few drops of triethylamine) to afford (2R,3S,4R,5S,6R)-3,4,5-tris(benzyloxy)-6-(trifluoromethyl)tetrahydro-2H-pyran-2-yl-2,2,2-trifluoro-N-phenylacetimidate (compound 71) (460 mg, 43% yield). LC-MS m/z=660.3 (M+1); 682.3 (M+Na).

Synthesis of compound 72: Compound 71 (460 mg, 0,70 mmol, 1.0 equiv.) and compound 46 (D. Schwizer et al., Chemistry, 18, 1342-51 (2012)) (216 mg, 0.84 mmol; 1.2 equiv.) were co-evaporated with toluene (3×), dried in vacuo for 1 h, and added to a suspension of flame-dried powdered 4 Å molecular sieves (0.4 g) in CH₂Cl₂ (10 mL). At −50° C., trimethylsilyltrifluoromethanesulfonate (10 μL, 0.07 mmol; 0.1 equiv.) was added, and the solution was warmed to rt overnight, quenched with triethylamine (1.0 mL) and concentrated. The residue was co-evaporated twice with toluene and purified by flash column chromatography on silica gel (hexanes/ethyl acetate) to afford tert-butyl(((1R,2R,3S)-3-ethyl-2-(((2R,3S,4R,5S,6R)-3,4,5-tris(benzyloxy)-6-(trifluoromethyl)tetrahydro-2H-pyran-2-yl)oxy)cyclohexyl)oxy)dimethylsilane (compound 72) (206 mg, 40% yield) as an α/β mixture. LC-MS ink 729.4 (M+1); 751.4 (M+Na).

Alternative Synthesis of compound 47: To a solution of compound 72 (206 mg, 0.28 mmol; 1.0 equiv.) in anhydrous THF (2 mL) was added tetrabutylammonium fluoride solution (1 M in THF; 1.4 mL, 1.4 mmol; 5.0 equiv) at room temperature. The resultant mixture was warmed to 55 CC and stirred for 18 h at 55° C., after which the reaction mixture was concentrated to half its volume. Water (10 mL) and. CH₂Cl₂ (10 mL) were added. The phases were separated and the aqueous phase was extracted with CH₂Cl₂. The combined organic phases were washed successively with 10% aq. NaHCO3 and water, dried over Na₂SO₄ and concentrated. Purification by silica gel chromatography using hexanes/ethyl acetate provided pure α/β anomer (1R,2R,3S)-3-ethyl-2-(((2R,3S,4R,5S,6R)-3,4,5-tris(benzyloxy)-6-(trifluoromethyl)tetrahydro-2H-pyran-2-yl)oxy)cyclohexan-1-ol (compound 47) (120 mg, 69% yield). LC-MS m/z=615.3 (M+1); 637.3 (M+Na).

Synthesis of compound 74: To a solution of compound 73 (102 mg, 0.13 mmol; 1.0 equiv; described in WO2005/054264), diphenyl sulfoxide (74 mg, 0.37 mmol; 2.8 equiv), 2,6-di-tert-butyl-4-methylpyridine (94 μL, 0.39 mmol; 3.0 equiv) in dichloromethane (2 mL) was added at −60° C. trifluoromethanesulfonic anhydride (31 μL, 0.18 mmol; 1.4 equiv). The reaction mixture was stirred for 5 min, after which a solution of compound 47 (120 mg, 0.2 mmol; 1.5 equiv) in dichloromethane (1 mL) was added. The mixture was stirred at −60° C. for 1 h, after which it was slowly warmed to room temperature overnight and quenched by the addition of saturated aqueous NaHCO₃. The organic layer was washed with brine, dried over Na₂SO₄, filtered and concentrated. The residue was purified by flash column chromatography on silica gel (hexanes/ethyl acetate) to afford (2R,3S,4S,5R,6R)-2-((benzoyloxy)methyl)-4-4(S)-1-(benzyloxy)-3-cyclohexyl-1-oxopropan-2-yl)oxy)-6-(((1R,2R,3S)-3-ethyl-2-(((2R,3S,4R,5S,6R)-3,4,5-tris(benzyloxy)-6-(trifluoromethyl)tetrahydro-2H-pyran-2-ypoxy)cyclohexyl)oxy)tetrahydro-2H-pyran-3,5-diyl dibenzoate (compound 74) (68 mg, 39% yield). LC-MS m/z=1333.6 (M+1); 1355.6 (M+Na).

Synthesis of compound 75: Compound 74 (68 mg, 0.05 mmol), Pearlman's catalyst (10 mg) and THF (5 mL) were added to a 20 mL glass vial. After the vial was purged with hydrogen (3 times), the reaction mixture was stirred at rt for 2 h under a hydrogen balloon at atmospheric pressure. The catalyst was removed by filtration through Celite® and the filtrate was concentrated under reduced pressure to give crude (S)-2-(((2R,3S,4S,5R,6R)-3,5-bis(benzoyloxy)-2-((benzoyloxy)methyl)-6-(((1R,2R,3S)-3-ethyl-2-(((2R,3,S,4R,5S,6R)-3,4,5-trihydroxy-6-(trifluoromethyptetrahydro-2H-pyran-2-yl)oxy)cyclohexyl)oxy)tetrahydro-2H-pyran-4-yl)oxy)-3-cyclohexylpropanoic acid that was directly used for the next step without further purification. LC-MS m/z=973.4 (M+1); 995.4 (M+Na).

To an ice-cold solution of crude (S)-2-(((2R,3S,4S,5R,6R)-3,5-bis(benzoyloxy)-2-((benzoyloxy)methyl)-6-(((1R,2R,3S)-3-ethyl-2-(((2R,3S,4R,5S,6R)-3,4,5-trillydroxy-6-(trifluoromethyl)tetrahydro-2H-pyran-2-yl)oxy)cyclohexyl)oxy)tetrahydro-2H-pyran-4-yl)oxy)-3-cyclohexylpropanoic acid (0.05 mmol) in methanol (2 mL) was added sodium methoxide solution (0.5 M in MeOH; 0.2 mL, 0.1 mmol; 2.0 equiv.). The reaction mixture was warmed to rt and allowed to stir for 2 h, after which it was concentrated to dryness. The resultant residue was purified by chromatography on silica gel with dichloromethane/methanol to afford sodium (5)-2-(((2R,3R,4S,5S,6R)-3-(benzoyloxy)-2-(((1R,2R,3S)-3-ethyl-2-(((2R,3S,4R,5S,6R)-3,4,5-trihydroxy-6-(trifluoromethyl)tetrahydro-2H-pyran-2-yl)oxy)cyclohexypoxy)-5-hydroxy-6-(hydroxymethyl)tetrahydro-2H-pyran-4-yl)oxy)-3-cyclohexylpropanoate (compound 75) (23 mg, 60% yield over two steps). LC-MS m/z=763.3 (M-1); 787.3 (M+Na). ¹H NMR (400 MHz, Methanol-d₄) δ ppm 8.13-8.03 (m, 2H), 7.66-7.56 (m, 1H), 7.49 (t, J=7.7 Hz, 2H), 5.37 (t, J=8.8 Hz, 1H), 5.20 (q, J=7.0 Hz, 1H), 5.13 (d, J=3.8 Hz, 1H), 4.65 (d, J=8.1 Hz, 1H), 4.19 (dd, J=3.4, 1.3 Hz, 1H), 4.03-3.92 (m, 2H), 3.85-3.69 (m, 4H), 3.58 (td, J=9.5, 8.7, 3.8 Hz, 3H), 3.31 (1H, overlapping with Methanol-d₄), 1.95 (d, J=11.6 Hz, 1H), 1.82 (dt, J=7.3, 3.8 Hz, 1H), 1.63 (s, 2H), 1.50 (td, J=9.7, 4.9 Hz, 3H), 1.42-1.10 (m, 8H), 1.09-0.97 (m, 1H), 0.97-0.78 (m, 5H), 0.74-0.47 (m, 4H).

### Example 13

### E-Selectin Activity—Binding Assay

The E-selectin_{LEC2}, construct was expressed and purified according to Preston et al., J. Mol. Cell Biol. (2015). The labeling procedure was performed by blocking the binding site with an excess of a suitable ligand to prevent the dye from reacting in the binding site. Subsequently the amine reactive protein labeling kit BLUE-NHS (Nanotemper Technologies GmbH, Munich, Germany) according to the protocol of the manufacturer. Unreacted dye was removed by dialysis against buffer A (10 mM HEPES, 150 mM NaCl, 1 mM CaCl₂, pH 7.4) using a Slide-A-Lyzer Dialysis cassette with 10 kDa cut-off (Thermo Scientific, Rockford, Ill., USA).

MEASUEMENT: All experiments were carried out using a Monolith™NT.115 device Nanoternper Technologies GmbH, Munich, Germany) at 298 K with 50% LED power, 30% laser power, initial delay of 5 seconds, laser on time of 30 seconds, and laser off time of 5 seconds with standard treated capillaries. Throughout all experiments the E-selectin_{LEC2} concentration was 0.1 μM. As starting point for the dilution series the concentration 30 times higher than the estimated K_{D} was chosen. The compounds were diluted 1:1 with buffer A supplemented with 0.1% (v/v) Tween 20.

### E-Selectin Antagonist Activity of Compounds

| Compound | K_{D} _{MST} [μM] |
|---|---|
| 27 | 879 |
| 28 | 161 |
| 33 | 55.6 |
| 37 | 21.5 |
| 38 | 7.2 |
| 41 | 5.3 |
| 42 | 2.4 |
| 51 | <1 |
| 53 | 0.7 |
| 59 | 0.3 |
| 69 | 0.6 |

DATA ANALYSIS: Every measurement was conducted once. Data points were normalized using the bound and unbound borders calculated by NanoTemper Analysis 1.2.205 software (NanoTemper Technologies GmbH, Munich, Germany) and analyzed with GraphPad Prism 5.0 (GraphPad Software, La Jolla, Calif., USA). To evaluate the KD of the compounds the measurements were globally fitted using equation (1) for single site binding. A. Cooper, Biophysical Chemistry, Royal Society of Chemistry, Cambridge, 2004, Ed 1, 109-10. Equation 1 is:

$$[ PL ] = \frac{{C}_{P} + {C}_{L} + {K}_{D} - \sqrt{{( {C}_{P} + {C}_{L} + {K}_{D} )}^{2} - 4} {C}_{P} {C}_{L}}{2 {C}_{P}} \qquad ( I )$$

where [PL] is the protein-ligand complex concentration and KD is the dissociation constant. CP represents the total concentration of protein and CL the total concentration of ligand.

### Example 14

### E-Selectin Activity—Alternative Binding Assay

E-selectin/Ig chimera was immobilized in 96 well microtiter plates by incubation at 37° C. for 2 hours. To reduce nonspecific binding, bovine serum albumin was added to each well and incubated at room temperature for 2 hours. The plate was washed and serial dilutions of the test compounds were added to the wells in the presence of conjugates of biotinylated, sLe^{a} polyacrylamide with streptavidin/horseradish peroxidase and incubated for 2 hours at room temperature.

To determine the amount of sLe^{a} hound to immobilized E-selectin after washing, the peroxidase substrate, 3,3′,5,5′ tetrametbylbenzidine (TMB) was added. After 3 minutes, the enzyme reaction was stopped by the addition of H₃PO₄, and the absorbance of light at a wavelength of 450 nm was determined. The concentration of test compound required to inhibit binding by 50% was determined and reported as the IC₅₀ value as shown in the table below.

### E-Selectin Antagonist Activity of Compound

| Compound | IC₅₀ [μM] |
|---|---|
| 75 | 0.7 |

1-51. (canceled)

52. At least one compound chosen from glycomimetic E-selectin antagonists having the following Formula: 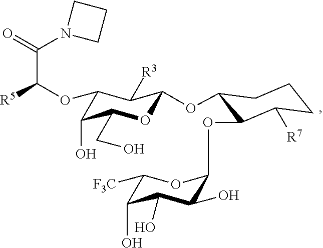
prodrugs thereof, and pharmaceutically acceptable salts of any of the foregoing, wherein
R³ is chosen from H, —OH, F, Cl, C₁₋₁₂ alkyl, —OY¹, and —OC(═O)Y¹ groups, wherein Y¹ is chosen from C₁₋₁₂ alkyl, C₂₋₁₂ alkenyl, C₂₋₁₂ alkynyl, and C₆₋₁₈ aryl groups;
R⁵ is chosen from C₁₋₁₈ alkyl, C₇₋₁₈ arylalkyl, and C₂₋₁₈ alkoxyalkyl groups; and
R⁷ is chosen from H, C₁₋₈ alkyl, and C₇₋₁₂ arylalkyl groups.

53. The at least one compound according to claim 52, wherein R⁷ is H.

54. The at least one compound according to claim 52, wherein R⁷ is methyl.

55. The at least one compound according to claim 52, wherein R⁷ is ethyl.

56. The at least one compound according to claim 52, wherein R⁷ is benzyl.

57. The at least one compound according to claim 52, wherein R³ is H.

58. The at least one compound according to claim 52, wherein R³ is OH.

59. The at least one compound according to claim 52, wherein R³ is F.

60. The at least one compound according to claim 52, wherein R³ is Cl.

61. The at least one compound according to claim 52, wherein R³ is chosen from —OY¹ groups.

62. The at least one compound according to claim 52, wherein R³ is —OMe.

63. The at least one compound according to claim 52, wherein R³ is —OPh.

64. The at least one compound according to claim 52, wherein R³ is chosen from —OC(═O)Y¹ groups.

65. The at least one compound according to claim 52, wherein R³ is from —OC(═O)Me.

66. The at least one compound according to claim 52, wherein R³ is from —OC(═O)Ph.

67. The at least one compound according to claim 1, wherein R⁵ is chosen from

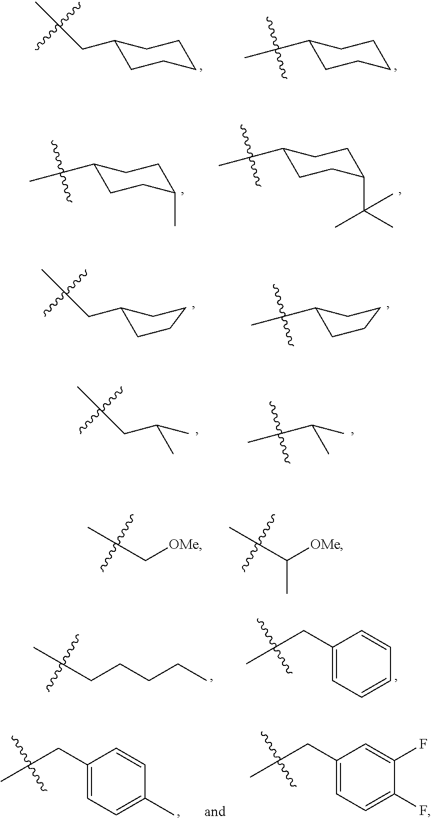

68. The at least one compound according to claim 52, wherein R⁵ is.

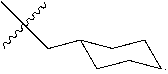 [SMILES: *CC1CCCCC1]

69. A pharmaceutical composition comprising the at least one compound of claim 52 and optionally at least one pharmaceutically acceptable excipient.

70. A method for treatment and/or prevention of at least one disease, disorder, and/or condition where inhibition of E-selectin mediated functions is useful, the method comprising administering to a subject in need thereof an effective amount of at least one compound of claim 52.

71. A method for treatment and/or prevention of at least one inflammatory disease, disorder, and/or condition, the method comprising administering to a subject in need thereof an effective amount of at least one compound of claim 52.

72. A method for treatment and/or prevention of metastasis of cancer cells, the method comprising administering to a subject in need thereof an effective amount of at least one compound of claim 52.

73. A method for inhibiting infiltration of cancer cells into bone marrow, the method comprising administering to a subject in need thereof an effective amount of at least one compound of claim 52.

74. A method for inhibiting adhesion of a tumor cell that expresses a ligand of E-selectin to an endothelial cell expressing E-selectin, the method comprising contacting the endothelial cell with an effective amount of at least one compound of claim 52.

75. The method according to claim 74, wherein the endothelial cell is present in bone marrow.

76. A method for treatment and/or prevention of thrombosis, the method comprising administering to a subject in need thereof an effective amount of at least one compound of claim 52.

77. A method for treatment and/or prevention of cancer, the method comprising administering to a subject in need thereof (a) an effective amount of at least one compound according to claim 52 and (b) at least one of therapy chosen from (i) chemotherapy and (ii) radiotherapy.

78. A method for enhancing hematopoietic stem cell survival, the method comprising administering to a subject in need thereof an effective amount of at least one compound of claim 52.

79. The method according to claim 78, wherein the subject has cancer and has received or will receive chemotherapy and/or radiotherapy.

80. A method for treatment and/or prevention of mucositis, the method comprising administering to a subject in need thereof an effective amount of at least one compound of claim 52.

81. The method according to claim 80, wherein the mucositis is oral mucositis, esophageal mucositis, and/or gastrointestinal mucositis.

82. The method according to claim 80, wherein the subject is afflicted with head and neck, breast, lung, ovarian, prostate, lymphatic, leukemic, and/or gastrointestinal cancer.

83. A method for mobilizing cells from the bone marrow, the method comprising administering to a subject in need thereof an effective amount of at least one compound of claim 52.
